SEQUENCE LISTING (63 sequences)

SEQ ID NO: 1 - protein, 26 aa, Artificial sequence
  Synthetic peptide
KWKSFLKTFKSAVKTVLHTALKAISS

SEQ ID NO: 2 - protein, 26 aa, Artificial sequence
  Synthetic peptide
KWKSFLKTFKSALKTVLHTALKAISS

SEQ ID NO: 3 - protein, 26 aa, Artificial sequence
  Synthetic peptide
KWKSFLKTFKSAVKTVLHTALKAISS

SEQ ID NO: 4 - protein, 26 aa, Artificial sequence
  Synthetic peptide
KWKSFLKTFKSAAKTVLHTALKAISS

SEQ ID NO: 5 - protein, 26 aa, Artificial sequence
  Synthetic peptide
KWKSFLKTFKSASKTVLHTALKAISS

SEQ ID NO: 6 - protein, 26 aa, Artificial sequence
  Synthetic peptide
KWKSFLKTFKSAKKTVLHTALKAISS

SEQ ID NO: 7 - protein, 26 aa, Artificial sequence
  Synthetic peptide
KWKSFLKTFKSALKTVLHTALKAISS

SEQ ID NO: 8 - protein, 26 aa, Artificial sequence
  Synthetic peptide
KWKSFLKTFKSAVKTVLHTALKAISS

SEQ ID NO: 9 - protein, 26 aa, Artificial sequence
  Synthetic peptide
KWKSFLKTFKSAAKTVLHTALKAISS

SEQ ID NO: 10 - protein, 26 aa, Artificial sequence
  Synthetic peptide
KWKSFLKTFKSASKTVLHTALKAISS

SEQ ID NO: 11 - protein, 26 aa, Artificial sequence
  Synthetic peptide
KWKSFLKTFKSAKKTVLHTALKAISS

SEQ ID NO: 12 - protein, 26 aa, Artificial sequence
  Synthetic peptide
KWKSFLKTFKSAGKTVLHTALKAISS

SEQ ID NO: 13 - protein, 26 aa, Artificial sequence
  Synthetic peptide
KWKSFLKTFKLAVKTVLHTALKAISS

SEQ ID NO: 14 - protein, 26 aa, Artificial sequence
  Synthetic peptide
KWKSFLKTFKAAVKTVLHTALKAISS

SEQ ID NO: 15 - protein, 26 aa, Artificial sequence
  Synthetic peptide
KWKSFLKTFKSAVKTVLHTALKAISS

SEQ ID NO: 16 - protein, 26 aa, Artificial sequence
  Synthetic peptide
KWKSFLKTFKVAVKTVLHTALKAISS

SEQ ID NO: 17 - protein, 26 aa, Artificial sequence
  Synthetic peptide
KWKSFLKTFKKAVKTVLHTALKAISS

SEQ ID NO: 18 - protein, 26 aa, Artificial sequence
  Synthetic peptide
KWKSFLKTFKLAVKTVLHTALKAISS

SEQ ID NO: 19 - protein, 26 aa, Artificial sequence
  Synthetic peptide
KWKSFLKTFKAAVKTVLHTALKAISS

SEQ ID NO: 20 - protein, 26 aa, Artificial sequence
  Synthetic peptide
KWKSFLKTFKSAVKTVLHTALKAISS

SEQ ID NO: 21 - protein, 26 aa, Artificial sequence
  Synthetic peptide
KWKSFLKTFKVAVKTVLHTALKAISS

SEQ ID NO: 22 - protein, 26 aa, Artificial sequence
  Synthetic peptide
KWKSFLKTFKKAVKTVLHTALKAISS

SEQ ID NO: 23 - protein, 26 aa, Artificial sequence
  Synthetic peptide
KWKSFLKTFKGAVKTVLHTALKAISS

SEQ ID NO: 24 - protein, 26 aa, Artificial sequence
  Synthetic peptide
KWKSFLKTFKSAKKTVLHTALKAISS

SEQ ID NO: 25 - protein, 26 aa, Artificial sequence
  Synthetic peptide
KWKSFLKTFKSAAKTVLHTALKAISS

SEQ ID NO: 26 - protein, 18 aa, Artificial sequence
  Synthetic peptide
ELEKGGLEGEKGGKELEK

SEQ ID NO: 27 - protein, 26 aa, Artificial sequence
  Synthetic peptide
KWKSFLKTKKSAVKTVLHTALKAISS

SEQ ID NO: 28 - protein, 26 aa, Artificial sequence
  Synthetic peptide
KWKSKLKTFKSAVKTVLHTALKAISS

SEQ ID NO: 29 - protein, 26 aa, Artificial sequence
  Synthetic peptide
KWKSFLKTAKSAVKTVLHTALKAISS

SEQ ID NO: 30 - protein, 26 aa, Artificial sequence
  Synthetic peptide
KWKSALKTFKSAVKTVLHTALKAISS

SEQ ID NO: 31 - protein, 26 aa, Artificial sequence
  Synthetic peptide
KWKSFLKTFKSARKTVLHTALKAISS

SEQ ID NO: 32 - protein, 26 aa, Artificial sequence
  Synthetic peptide
KWKSFAKTFKSAVKTVLHTAAKAISS

SEQ ID NO: 33 - protein, 26 aa, Artificial sequence
  Synthetic peptide
KWKSFKKTFKSAVKTVLHTAKKAISS

SEQ ID NO: 34 - protein, 25 aa, Artificial sequence
  Synthetic peptide
WKSFLKTFKSAVKTVLHTALKAISS

SEQ ID NO: 35 - protein, 24 aa, Artificial sequence
  Synthetic peptide
KSFLKTFKSAVKTVLHTALKAISS

SEQ ID NO: 36 - protein, 24 aa, Artificial sequence
  Synthetic peptide
KWKSFLKTFKSAVKTVLHTALKAI

SEQ ID NO: 37 - protein, 23 aa, Artificial sequence
  Synthetic peptide
KWKSFLKTFKSAVKTVLHTALKA

SEQ ID NO: 38 - protein, 26 aa, Artificial sequence
  Synthetic peptide
KIKSALKTLKSFKKTAAHTLFKVWSS

SEQ ID NO: 39 - protein, 26 aa, Artificial sequence
  Synthetic peptide
SWSKFLKKFTKAKSHVLTTALSAIKK

SEQ ID NO: 40 - protein, 26 aa, Artificial sequence
  Synthetic peptide
KWKSFLKTFKXAXKTVLHTALKAISS

SEQ ID NO: 41 - protein, 26 aa, Artificial sequence
  Synthetic peptide
KHAVIKWSIKSSVKFKISTAFKATTI

SEQ ID NO: 42 - protein, 26 aa, Artificial sequence
  Synthetic peptide
HWSKLLKSFTKALKKFAKAITSVVST

SEQ ID NO: 43 - protein, 26 aa, Artificial sequence
  Synthetic peptide
KWKSFLKTFKXAVKTVLHTALKAISS

SEQ ID NO: 44 - protein, 26 aa, Artificial sequence
  Synthetic peptide
KWKSFLKTFKSAXKTVLHTALKAISS

SEQ ID NO: 45 - protein, 26 aa, Artificial sequence
  Synthetic peptide
KWKSFLKTFKSAKKTVLHTALKLISS

SEQ ID NO: 46 - protein, 26 aa, Artificial sequence
  Synthetic peptide
KWKSFLKTFKSLKKTVLHTALKAISS

SEQ ID NO: 47 - protein, 26 aa, Artificial sequence
  Synthetic peptide
KWKSFLKTFKSAKKTVLHTLLKAISS

SEQ ID NO: 48 - protein, 26 aa, Artificial sequence
  Synthetic peptide
KWKSFLKTFKSLKKTVLHTALKLISS

SEQ ID NO: 49 - protein, 26 aa, Artificial sequence
  Synthetic peptide
KWKSFLKTFKSLKKTVLHTLLKAISS

SEQ ID NO: 50 - protein, 26 aa, Artificial sequence
  Synthetic construct peptide
KWKSFLKTFKSLKKTVLHTLLKLISS

SEQ ID NO: 51 - protein, 22 aa, Artificial sequence
  Synthetic construct peptide
KSFLKTFKSAKLKTVLHTALKA

SEQ ID NO: 52 - protein, 22 aa, Artificial sequence
  Synthetic construct  peptide
KSFLKTFKSAKLKTVLHTALKA

SEQ ID NO: 53 - protein, 26 aa, Artificial Sequence
  Synthetic construct  antimicrobial peptide D2
KWKSFLKTFKSAKKTVLHTLLKAISS

SEQ ID NO: 54 - protein, 26 aa, Artificial Sequence
  Synthetic construct   antimicrobial peptide D3
KWKSFLKTFKSLKKTVLHTLLKAISS

SEQ ID NO: 55 - protein, 26 aa, Artificial Sequence
  Synthetic construct  antimicrobial peptide D4
KWKSFLKTFKSLKKTVLHTLLKLISS

SEQ ID NO: 56 - protein, 26 aa, Artificial Sequence
  Synthetic construct  antimicrobial peptide D5
KWKSFLKTFKSLKKTKLHTLLKLISS

SEQ ID NO: 57 - protein, 26 aa, Artificial Sequence
  Synthetic construct antimicrobial peptide D6
KWKSFLKTFKSLKKTKLHTLLKVISS

SEQ ID NO: 58 - protein, 26 aa, Artificial Sequence
  Synthetic construct  antimicrobial peptide D7
KWKSFLKTFKSVKKTKLHTLLKLISS

SEQ ID NO: 59 - protein, 26 aa, Artificial Sequence
  Synthetic construct  antimicrobial peptide D8
KWKSFLKTFKSVKKTKLHTLLKVISS

SEQ ID NO: 60 - protein, 26 aa, Artificial Sequence
  Synthetic construct  antimicrobial peptide D9
KWKSFLKTFKSLKKTKLHTLLKAISS

SEQ ID NO: 61 - protein, 26 aa, Artificial Sequence
  Synthetic construct  antimicrobial peptide D10
KWKSFLKTFKSAKKTKLHTLLKLISS

SEQ ID NO: 62 - protein, 26 aa, Artificial Sequence
  Synthetic construct
KWKSFLKTFKSXXKTXLHTXLKXISS

SEQ ID NO: 63 - protein, 10 aa, Artificial Sequence
  Synthetic construct  model random coil peptide
XGAKGAGVGL

(19) United States
(12) Patent Application Publication
(10) Pub. No.: US 2010/0099614 A1
(43) Pub. Date: Apr. 22, 2010

(54) Antimicrobial Peptides and Methods of Use

(76) Inventors: Robert S. Hodges, Denver, CO (US); Ziqing Jiang, Denver, CO (US)

Correspondence Address: GREENLEE WINNER AND SULLIVAN P C, 4875 PEARL EAST CIRCLE, SUITE 200, BOULDER, CO, 80301, US

(21) Appl. No.: 12/574,545

(22) Filed: Oct. 6, 2009

Related U.S. Application Data
(60) Provisional application No. 61/195,299, filed on Oct. 6, 2008.

(51) Int. Cl. A61K 38/16; C07K 14/00; A61P 31/00; A61P 31/10; A61P 31/04; A01P 1/00; A01N 37/18

(52) U.S. Cl. 514/12; 530/324; 530/326; 530/325

(57) ABSTRACT

Disclosed herein are antimicrobial peptides with useful and/or superior properties such as specificity, resistance to degradation, antimicrobial activity, desirably low levels of hemolytic activity, and a therapeutic index against a broad range of microorganisms including gram-negative, gram-positive and acid-fast bacteria, fungi and other organisms. Also provided are pharmaceutical compositions comprising these peptides and methods of using such peptides to control microbial growth or to treat or reduce incidence of infections caused by such microorganisms. Also disclosed are peptides at least one or all amino acids in the D configuration. Compositions disclosed herein are useful in the treatment of bacterial, mycobacterial and/or fungal infections or for reducing microbial cell numbers or growth on surfaces or in materials.

### CROSS-REFERENCE TO RELATED APPLICATIONS

This application claims benefit of U.S. Provisional Application 61/195,299, filed Oct. 6, 2008, which application is incorporated by reference herein to the extent there is no inconsistency with the present disclosure.

### STATEMENT ON FEDERALLY SPONSORED RESEARCH OR DEVELOPMENT

This invention was made with government support under National Institute of Allergy and Infectious Diseases (NIAID) R01 AI067296 and R01 GM061855 awarded by the National Institutes of Health. The United States government has certain rights in the invention.

### BACKGROUND OF THE INVENTION

The present invention broadly relates to novel antimicrobial peptides and methods of making and using such peptides to inhibit microbial growth and in pharmaceutical compositions for treatment or prevention of infections caused by a broad range of microorganisms including, but not limited to, gram-positive and gram-negative bacteria, fungi, and mycobacterial pathogens including Mycobacterium tuberculosis.

The extensive clinical use of classical antibiotics has led to the growing emergence of many medically relevant resistant strains of bacteria (1,2). Only three new classes of antibiotics (oxazolidinone, linezolid, the streptogramins and the lipopeptide-daptomycin) have been introduced into medical practice in the past 40 years; therefore, there is need for new antibiotics. Cationic antimicrobial peptides could represent such a new class of antibiotics (3-5). Although the exact mode of action of the cationic antimicrobial peptides has not been established, all cationic amphipathic peptides interact with membranes. It has been proposed that the cytoplasmic membrane is the main target of some peptides, where peptide accumulation in the membrane may cause increased permeability and loss of barrier function (6,7). Therefore, the development of resistance to these membrane active peptides is less likely because this would require substantial changes in the lipid composition of cell membranes of microorganisms.

Two major classes of the cationic antimicrobial peptides are the α-helical and the β-sheet peptides (3,4,8,9). The β-sheet class includes cyclic peptides constrained in this conformation either by intramolecular disulfide bonds, e.g., defensins (10) and protegrins (11), or by an N-terminal to C-terminal covalent bond, e.g., gramicidin S (12) and tyrocidines (13). α-helical peptides are more linear molecules that mainly exist as disordered structures in aqueous media and as amphipathic helices upon interaction with the hydrophobic membranes. These include cecropins (14), magainins (15) and melittins (16).

The major barrier to the use of antimicrobial peptides as antibiotics is their potential toxicity to eukaryotic cells. This is perhaps not surprising if the target is indeed the cell membrane (3-6). To be useful as a broad-spectrum antibiotic, it is necessary to dissociate toxic effects (including lytic activity) from antimicrobial activity, i.e., increase the antimicrobial activity and reduce toxicity to normal cells, especially in a human or other animal in need of treatment for an infection.

A synthetic peptide approach to examining the effect of changes, including incremental changes in hydrophobicity or hydrophilicity, amphipathicity and helicity of cationic antimicrobial peptides can facilitate rational design of peptide antibiotics. Generally, only L-amino acids are the isomers found throughout natural peptides and proteins; D-amino acids are the isomeric forms rarely seen in natural peptides/proteins except in some bacterial cell walls. In certain circumstances, the helix-destabilizing properties of D-amino acids allow the controlled alteration of the hydrophobicity, amphipathicity, and helicity of amphipathic α-helical peptides and also reduce degradation by host or microbial proteases.

The structural framework of an amphipathic α-helical antimicrobial peptide (SEQ ID NO:1), V₆₈₁ (28), was systematically changed to alter peptide amphipathicity, hydrophobicity and helicity by single D- or L-amino acid substitutions in the center of either the polar or nonpolar faces of the amphipathic helix has been described (WO 2006/065977). Peptide V₆₈₁ has excellent antimicrobial activity and strong hemolytic activity (27,28). It was found that hydrophobicity, amphipathicity and helicity have dramatic effects on the biophysical and biological activities as well as antimicrobial activity and specificity. Self-association also affects the biological activities of amphipathic α-helical antimicrobial peptides.

Fungal infections can range from superficial and cutaneous to deeply invasive and disseminated. Human mycoses include aspergillosis, blastomycosis, candidiasis, coccidioidomycosis, cryptococcosis, histoplasmosis, paracoccidiomycosis, sporotrichosis and zygomycosis. Fungal infections occur more frequently in people whose immune systems are suppressed, who have been treated with broad-spectrum antibacterial agents, or who have been subjected to invasive procedures (99). Fungal infections are the major cause of morbidity and mortality in patients with organ transplantation, cancer chemotherapy and the human immunodeficiency virus (HIV) (100-102). Candida and Aspergillus account for more than 80% of fungal infections in patients with solid-organ transplantation (100). The systemic mycoses (cryptococcosis, histoplasmosis, and sporotrichosis) and superficial and mucocutaneous mycoses (candidiasis and dermatophytosis) are common fungal infections in HIV patients (102). Candida, Aspergillus, Rhizopus and Cryptococcus neoformans are common fungal pathogens in cancer patients (101).

There are fewer antifungal than antibacterial drugs (99), in part because fungi are eukaryotes. Thus, many agents that inhibit fungal protein, RNA, or DNA biosynthesis do the same in the mammalian cells, producing toxic side effects in patients. Because there is an increase in the occurrence of resistant pathogenic fungal strains (100), the development of a new antifungal antibiotics is critical. Cationic antimicrobial peptides (AMPs) generally have unusually broad spectra of “antimicrobial” activity, especially against fungi (including yeasts), which make them important candidates as antifungal therapeutic agents.

Although the exact mode of action of antimicrobial peptides has not been established, it is believed that the cytoplasmic membrane is the main target of many antimicrobial peptides, with peptide accumulation in the membrane causing increased permeability and loss of barrier function, resulting in the leakage of cytoplasmic components and cell death. Polyene antibiotics kill fungi by this same mechanism. Cationic AMPs of the α-helical class have two unique features: a net positive charge of at least +2 and an amphipathic character, with a non-polar face and a polar/charged face (3,103). Factors believed to be important for antimicrobial activity include peptide hydrophobicity, the presence of positively charged residues, an amphipathic nature that segregates basic and hydrophobic residues, and secondary structure. Peptides with mainly antifungal activity, e.g., some isolated from plants, are generally rich in polar and neutral amino acids, which suggests a unique structure-activity relationship (104). There are no obvious conserved structural domains that give rise to antifungal activity, and the mechanism of action of some antifungal peptides is still not clear (105).

The V681 peptide (Cecropin A (1-8)+Melittin B (1-18) derivative) was studied as to what features of an α-helical antimicrobial peptide could be changed to control specificity between prokaryotic and eukaryotic cells, retain antimicrobial activity and reduce hemolytic activity for human red blood cells (53, WO 2006/065977). A single valine to lysine substitution in the center of the non-polar face dramatically reduced toxicity and increased therapeutic index (53). The sole target of this peptide was the membrane (92). D- and L-peptides had equal activities, suggesting that the antimicrobial mechanism did not involve a stereoselective interaction with a chiral enzyme, lipid or protein receptor (92), and the all-D peptide was resistant to proteolytic enzyme degradation, thus enhancing its potential as a clinical therapeutic. An optimum hydrophobicity of the non-polar face gave the best therapeutic index (93). Increased hydrophobicity beyond this optimum dramatically reduced antimicrobial activity and increased peptide self-association (93). Net charge and the number of positively charged residues on the polar face are important for antimicrobial activity and hemolytic activity (106).

The list of factors important for antimicrobial activity include lack of secondary structure in benign (non-denaturing) medium and induced structure in the hydrophobic environment of the membrane; a positively-charged residue in the center of the non-polar face of amphipathic cyclic β-sheet and α-helical peptides as a determinant for locating the peptides to the interfacial region of prokaryotic membranes and decreasing transmembrane penetration into eukaryotic membranes; and limited peptide self-association in an aqueous environment (WO 2006/065977, 53,92-93,30,19).

As described herein, the all D-form of substituted variants of the V13K antimicrobial peptide (SEQ ID NO:24) were studied with respect to the effect of hydrophobicity on antifungal activity toward pathogenic fungi including, but not limited to, Aspergillus nidulans, Absidia corymbifera, Rhizomucor spp., Rhizopus microsporus, Rhizopus oryzae, Scedosporium prolificans and Candida albicans. Surprisingly, hydrophobicity had significant and different effects on antifungal activity depending on the class of fungi. In Zygomycota fungi, increasing hydrophobicity decreased antifungal activity, whereas increasing hydrophobicity increased antifungal activity for Ascomycota fungi.

In addition, with the recent re-emergence of tuberculosis and significant incidence of antibiotic resistant strains, there is a need to identify effective new antimycobacterial agents. Thus, there is a need for new classes of antimycobacterial agents with different modes of action than classical antibiotics such as rifampin and isoniazid. There is also a long felt need in the art for new antimicrobial agents, especially those which are active against recalcitrant microorganisms such as pathogenic fungi as well as a wide variety of bacterial pathogens, including mycobacterial pathogens.

### SUMMARY OF THE INVENTION

Regardless of the ultimate correctness of any mechanistic explanation or hypothesis set forth herein, the compositions and methods of the invention can be operative and useful.

The present invention provides peptides which are useful as antimicrobial agents and in methods of inhibiting microbial growth, especially fungi and mycobacteria, using compositions comprising such antimicrobial agents in effective amounts. In embodiments of the invention, the antimicrobial peptides range in size from about 21 or about 22 to about 28 amino acids in length, or from about 22 to about 26 amino acids in length, the amino acids being joined by peptide bonds and having a core of about 21 amino acids. The core comprises an amino acid sequence as given in SEQ ID NO:62, amino acids 5 to 24, or amino acids 5 to 24 of any of SEQ ID NOs:53-61, or of SEQ ID NOs:56-61, for example. The amino acids in the peptides can be all in the L configuration, all in the D configuration or in a combination of D and L configurations. The peptides can have a blocking group at the N-terminus, such as an acetyl group or a polyethylene glycol moiety. The peptide can have an amide or a carboxyl moiety at the C-terminus. The peptides of the present invention have potent antimicrobial activities and are useful against bacteria, fungi, viruses, and protozoa. The peptides are generally effective of any organism having a cellular or structural component of a lipid bilayer membrane. These peptides are useful as human and/or veterinary therapeutics or as antimicrobial agents in agricultural, medical, food science or industrial applications.

Without wishing to be bound by any particular theory, it is believed that factors affecting antimicrobial activity include, without limitation, the presence of both hydrophobic and basic residues, an amphipathic nature that segregates basic and hydrophobic residues, and an inducible or preformed secondary structure (α-helical or β-sheet). Also without wishing to be bound by any particular theory, it is believed that by substituting certain D-amino acids into the center of the hydrophobic face of an amphipathic α-helical model peptide, disruption of α-helical structure can occur. Although different D-amino acids can disrupt α-helical structure to different degrees, the destabilized structure is induced to fold into an α-helix in a hydrophobic medium. Advantages of substituting single D- or L-amino acid substitutions at a specific site are opportunity for greater understanding of the mechanism of action of these peptides and advantageous properties can be identified.

Provided is a method of treating a patient (a human or animal patient suffering from a microbial infection or susceptible to a microbial infection or exposed to an infectious microorganism) comprising administering to the patient a peptide as disclosed herein, for example a method of treating a microbial infection, reducing the incidence of infection or lessening the severity of an infection, if contracted. In a particular embodiment, the microbial infection involves one or more of a bacterium, including but not limited to a mycobacterium, for example, Mycobacterium tuberculosis, a virus, a fungus (ascomycete or zygomycete, for example), or a protozoan. In a particular embodiment, the microbial infection involves one or more kinds of microorganisms, e.g. two different kinds of bacteria, a bacterium and a fungus, and so forth. The peptide can be one matching amino acids 3-24 or any 19 amino acid sequence therein or 1 to 26 of the consensus sequence provided herein in SEQ ID NO:62, or of any of SEQ ID NOs:53-61, or it can be one of SEQ ID NO:53-61 or 56-61, advantageously that of SEQ ID NO:56. SEQ ID NO:56 is especially useful against M. tuberculosis. The peptide can be modified at the N-terminus and/or it can have at the C-terminus an amide or a carboxyl group, and one or all of the amino acids can be L or D amino acids.

In an embodiment, there is provided a method for increasing antimicrobial activity of a peptide. In an embodiment, there is provided a method for decreasing hemolytic activity of a peptide while maintaining antimicrobial activity or while minimizing a reduction of antimicrobial activity, especially by amino acid substitutions, advantageously positively charged amino acids, on the nonpolar face of a helical antimicrobial peptide. In an embodiment, provided is a method of increasing or maintaining antimicrobial activity and decreasing hemolytic activity of a peptide (or minimizing a reduction of antimicrobial activity).

The antimicrobial peptides disclosed herein, with proper control of alteration of the hydrophobicity and/or hydrophilicity, amphipathicity and helicity of an α-helical peptide, have useful and/or improved biological activity and specificity (e.g. improved therapeutic index). Exemplified are peptides derived by altering the amino acid sequence of the 26-residue D1 peptide (SEQ ID NO: 24) (for example, those of SEQ ID NOs:53-62 or of SEQ ID NOs:56-61). The terms “derived from” or “derivative” are meant to indicate that such peptides are the same or shorter than the D1 peptide in size and have one or more amino acid residues substituted, or a combination of both; further variations are also described herein, for example in SEQ ID NO:62. The D1 peptide (SEQ ID NO:24) was varied with respect to sequence at certain positions to study the effects of peptide hydrophobicity and/or hydrophilicity, amphipathicity and helicity on biological activities, for example antimicrobial and hemolytic activities, by substituting one or more amino acid residues at certain locations. The D5 peptide (SEQ ID NO:56) was identified as having a desirable therapeutic index, and surprisingly, significant antimicrobial activity against M. tuberculosis.

In an embodiment, there are provided compositions and methods relating to an antimicrobial peptide characterized by an amino acid sequence selected from the group consisting of SEQ ID NOS:53-62, and other peptides as disclosed herein. Note that SEQ ID NO:1, peptide V681, is equivalent to SEQ ID NOS:3 and 15. Table 1 includes peptides with substitutions on the nonpolar face at position X=13 and on the polar face at position X=11. Table 2 includes other peptide analogs.

TABLE 1
Summary of partial sequence listing information.

| | Peptide | Amino Acid Position | | | | | | | | | | | | | | | | | | | | | | | | | |
|---|---|---|---|---|---|---|---|---|---|---|---|---|---|---|---|---|---|---|---|---|---|---|---|---|---|---|---|
| SEQ ID NO: | Name | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
| Enantiomer* | | L | L | L | L | L | L | L | L | L | L | L | L | L | L | L | L | L | L | L | L | L | L | L | L | L | L |
| 1 | V681 | K | W | K | S | F | L | K | T | F | K | S | A | V | K | T | V | L | H | T | A | L | K | A | I | S | S |
| 2 | NL_{L} | K | W | K | S | F | L | K | T | F | K | S | A | L_{L} | K | T | V | L | H | T | A | L | K | A | I | S | S |
| 3 | NV_{L} | K | W | K | S | F | L | K | T | F | K | S | A | V_{L} | K | T | V | L | H | T | A | L | K | A | I | S | S |
| 4 | NA_{L} | K | W | K | S | F | L | K | T | F | K | S | A | A_{L} | K | T | V | L | H | T | A | L | K | A | I | S | S |
| 5 | NS_{L} | K | W | K | S | F | L | K | T | F | K | S | A | S_{L} | K | T | V | L | H | T | A | L | K | A | I | S | S |
| 6 | NK_{L} | K | W | K | S | F | L | K | T | F | K | S | A | K_{L} | K | T | V | L | H | T | A | L | K | A | I | S | S |
| 7 | NL_{D} | K | W | K | S | F | L | K | T | F | K | S | A | L_{D} | K | T | V | L | H | T | A | L | K | A | I | S | S |
| 8 | NV_{D} | K | W | K | S | F | L | K | T | F | K | S | A | V_{D} | K | T | V | L | H | T | A | L | K | A | I | S | S |
| 9 | NA_{D} | K | W | K | S | F | L | K | T | F | K | S | A | A_{D} | K | T | V | L | H | T | A | L | K | A | I | S | S |
| 10 | NS_{D} | K | W | K | S | F | L | K | T | F | K | S | A | S_{D} | K | T | V | L | H | T | A | L | K | A | I | S | S |
| 11 | NK_{D} | K | W | K | S | F | L | K | T | F | K | S | A | K_{D} | K | T | V | L | H | T | A | L | K | A | I | S | S |
| 12 | NG | K | W | K | S | F | L | K | T | F | K | S | A | G | K | T | V | L | H | T | A | L | K | A | I | S | S |
| 13 | PL_{L} | K | W | K | S | F | L | K | T | F | K | L_{L} | A | V | K | T | V | L | H | T | A | L | K | A | I | S | S |
| 14 | PA_{L} | K | W | K | S | F | L | K | T | F | K | A_{L} | A | V | K | T | V | L | H | T | A | L | K | A | I | S | S |
| 15 | PS_{L} | K | W | K | S | F | L | K | T | F | K | S_{L} | A | V | K | T | V | L | H | T | A | L | K | A | I | S | S |
| 16 | PV_{L} | K | W | K | S | F | L | K | T | F | K | V_{L} | A | V | K | T | V | L | H | T | A | L | K | A | I | S | S |
| 17 | PK_{L} | K | W | K | S | F | L | K | T | F | K | K_{L} | A | V | K | T | V | L | H | T | A | L | K | A | I | S | S |
| 18 | PL_{D} | K | W | K | S | F | L | K | T | F | K | L_{D} | A | V | K | T | V | L | H | T | A | L | K | A | I | S | S |
| 19 | PA_{D} | K | W | K | S | F | L | K | T | F | K | A_{D} | A | V | K | T | V | L | H | T | A | L | K | A | I | S | S |
| 20 | PS_{D} | K | W | K | S | F | L | K | T | F | K | S_{D} | A | V | K | T | V | L | H | T | A | L | K | A | I | S | S |
| 21 | PV_{D} | K | W | K | S | F | L | K | T | F | K | V_{D} | A | V | K | T | V | L | H | T | A | L | K | A | I | S | S |
| 22 | PK_{D} | K | W | K | S | F | L | K | T | F | K | K_{D} | A | V | K | T | V | L | H | T | A | L | K | A | I | S | S |
| 23 | PG | K | W | K | S | F | L | K | T | F | K | G | A | V | K | T | V | L | H | T | A | L | K | A | I | S | S |
| Enantiomer | | D | D | D | D | D | D | D | D | D | D | D | D | D | D | D | D | D | D | D | D | D | D | D | D | D | D |
| 24 | D-NK_{D} | K | W | K | S | F | L | K | T | F | K | S | A | K | K | T | V | L | H | T | A | L | K | A | I | S | S |
| 25 | D-NA_{L} | K | W | K | S | F | L | K | T | F | K | S | A | A_{L} | K | T | V | L | H | T | A | L | K | A | I | S | S |
| 1 | D-V681 | K | W | K | S | F | L | K | T | F | K | S | A | V | K | T | V | L | H | T | A | L | K | A | I | S | S |

*L-enantiomer unless otherwise indicated in the Enantiomer column or subscript.

TABLE 2
Summary of partial sequence listing information.

| SEQ | | | | | | | | | | | | | | | | | | | | | | | | | | | |
|---|---|---|---|---|---|---|---|---|---|---|---|---|---|---|---|---|---|---|---|---|---|---|---|---|---|---|---|
| ID | | Amino Acid Position | | | | | | | | | | | | | | | | | | | | | | | | | |
| NO: | Peptide Name | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 |
| | Enantiomer* | L | L | L | L | L | L | L | L | L | L | L | L | L | L | L | L | L | L | L | L | L | L | L | L | L | L |
| 1 | V681 | K | W | K | S | F | L | K | T | F | K | S | A | V | K | T | V | L | H | T | A | L | K | A | I | S | S |
| 27 | F9 to K9 | K | W | K | S | F | L | K | T | K | K | S | A | V | K | T | V | L | H | T | A | L | K | A | I | S | S |
| 28 | F5 to K5 | K | W | K | S | K | L | K | T | F | K | S | A | V | K | T | V | L | H | T | A | L | K | A | I | S | S |
| 29 | F9 to A_{D}9 | K | W | K | S | F | L | K | T | A_{D} | K | S | A | V | K | T | V | L | H | T | A | L | K | A | I | S | S |
| 30 | F5 to A_{D}5 | K | W | K | S | A_{D} | L | K | T | F | K | S | A | V | K | T | V | L | H | T | A | L | K | A | I | S | S |
| 31 | V13 to R13 | K | W | K | S | F | L | K | T | F | K | S | A | R | K | T | V | L | H | T | A | L | K | A | I | S | S |
| 32 | L6-A_{D}6, | K | W | K | S | F | A_{D} | K | T | F | K | S | A | V | K | T | V | L | H | T | A | A_{D} | K | A | I | S | S |
| | L21-A_{D}21 | | | | | | | | | | | | | | | | | | | | | | | | | | |
| 33 | L6-K_{L}6, | K | W | K | S | F | K | K | T | F | K | S | A | V | K | T | V | L | H | T | A | K | K | A | I | S | S |
| | L21-K_{L}21 | | | | | | | | | | | | | | | | | | | | | | | | | | |
| 34 | Remove K1 | | W | K | S | F | L | K | T | F | K | S | A | K | K | T | V | L | H | T | A | L | K | A | I | S | S |
| 35 | Remove K1, | | | K | S | F | L | K | T | F | K | S | A | K | K | T | V | L | H | T | A | L | K | A | I | S | S |
| | W2 | | | | | | | | | | | | | | | | | | | | | | | | | | |
| 36 | Remove S25, | K | W | K | S | F | L | K | T | F | K | S | A | K | K | T | V | L | H | T | A | L | K | A | I | | |
| | S26 | | | | | | | | | | | | | | | | | | | | | | | | | | |
| 37 | Remove I24, | K | W | K | S | F | L | K | T | F | K | S | A | K | K | T | V | L | H | T | A | L | K | A | | | |
| | S25, S26 | | | | | | | | | | | | | | | | | | | | | | | | | | |
| 38 | non-polar face | K | I | K | S | AD | L | K | T | L | K | S | F | K | K | T | A | A | H | T | L | F | K | V | W | S | S |
| | shuffle | | | | | | | | | | | | | | | | | | | | | | | | | | |
| 39 | polar face | S | W | S | K | F | L | K | K | F | T | K | A | K | S | H | V | L | T | T | A | L | S | A | I | K | K |
| | shuffle | | | | | | | | | | | | | | | | | | | | | | | | | | |

*L-enantiomer unless otherwise indicated.

Advantageously, the peptide is helical in a hydrophobic environment. Circular dichroism spectroscopy can be used to monitor α-helical structure in 50% trifluoroethanol, which mimics the hydrophobic environment of the cytoplasmic membrane.

Certain peptides that are helical variants (analogs) with the desired biological activities have very little α-helical structure in a “benign” medium (a non-denaturing medium like 50 mM PO₄ buffer containing 100 mM KCl, pH 7) as determined by circular dichroism spectroscopy. This structural property can result in decreased dimerization (or aggregation) in benign medium and easier penetration of the cell wall to reach the cytoplasmic membrane of the microbe. Furthermore, disruption of the α-helical structure in benign medium can allow a positively-charged peptide to bind to the negatively-charged cell surface of the microbe (e.g. lipopolysaccharide, LPS), but the relative lack of structure can decrease the affinity of peptide for this surface and allow the peptide to more easily pass through the cell wall and enter the interface region of the membrane so that the peptide is parallel to the surface of membrane. Here the peptide can be induced by the hydrophobic environment of the membrane into its α-helical structure, where it is believed that the non-polar face of the amphiphilic peptide interacts with hydrophobic portions of the membrane, and its polar and positively-charged groups on the polar face interact with the negatively-charged groups of the phospholipids on the surface of the membrane. In an embodiment, a peptide is net positively-charged and amphipathic (amphiphilic) when in an α-helical structure.

Self-associating ability of certain peptide analogs was studied by temperature profiling in RP-HPLC from 5° C. to 80° C. in solution. Self association is an important parameter relative to antimicrobial and hemolytic activities. Generally, high ability to self-associate in solution was correlated with weak antimicrobial activity and strong hemolytic activity, and strong hemolytic activity of the peptides generally correlated with high hydrophobicity, high amphipathicity and high helicity. In most cases, the D-amino acid substituted peptides possessed an enhanced average antimicrobial activity compared with L-diastereomers. As illustrated herein, the therapeutic index of V₆₈₁ was improved 90-fold and 23-fold against gram-negative and gram-positive bacteria, respectively (using geometric mean comparison). By replacing the central hydrophobic or hydrophilic amino acid residue on the nonpolar or the polar face of these amphipathic molecules with a series of selected D- and L-amino acids, other antimicrobial peptides with enhanced activities were produced.

Herein, a subscripted D following an amino acid residue denotes that the residue is a D-amino acid residue; similarly a subscript L denotes an L-amino acid residue. Where there is no indication of D or L, the amino acid is in the L-configuration. In the peptide name, an initial D- (not subscripted) denotes all D-amino acids in the peptide except where specified (e.g. D-NA_{L} denotes all D-amino acids with the exception of a single substitution of L-Ala in the center of the non-polar face specified by N). The boxed residues denote the differences at position 13 in the sequence which is in the center of the non-polar face (see also FIG. 1A). The Ac-designation at the N-terminus of the peptide indicates acetylation, which improves resistance to degradation. Alternatively, an antimicrobial peptide of the present invention can be modified with other groups, for example, polyethylene glycol, which may improve solubility, inhibit aggregation and/or improve persistence in the body.

In an embodiment, a peptide of the invention is contained within a larger polypeptide or protein. In an embodiment, a peptide of the invention is covalently or non-covalently associated with another compound, including but not limited to a polymer, for example an amphiphilic polymer or copolymer to improve solubility and decrease the tendency of the peptide to aggregate (self-associate).

The peptides disclosed herein as SEQ ID NO:53-62, especially SEQ ID NO:56, have antimicrobial activity against a wide range of microorganisms, including fungi, gram-positive and gram-negative bacteria and the acid-fast bacteria, for example Mycobacteria such as M. tuberculosis. Detailed descriptions of the microorganisms belonging to gram-positive and gram-negative or other types of bacteria can be found, for example, in Medical Microbiology (1991), 3^{rd} edition, edited by Samuel Baron, Churchill Livingstone, N.Y. Examples of susceptible bacteria can include but are not limited to Mycobacteria, Escherichia coli, Salmonella typhimurium, Pseudomonas aeruginosa, Staphylococcus aureus, Staphylococcus epidermidis, Bacillus subtilis, Enterococcus faecalis, Corynebacterium xerosis, and Bacillus anthracis. The antimicrobial activities of the present peptides have been demonstrated herein against certain gram-positive and gram-negative bacteria. It is well known in the art that these bacteria are considered as model organisms for either gram-negative or gram-positive bacteria, and thus, any biological activity demonstrated against these model organisms is accepted as an indication of that activity against the range of gram-negative or gram-positive bacteria. Similarly, the D5 peptide exhibits significant antimicrobial activity against M. tuberculosis, reflecting activity against other members of the acid-fast bacteria (mycobacteria, nocardia, and the like). Certain peptides are active against fungi including, but not limited to, Candida albicans, A. nidulans, A. corymbifera, Rhizomucor spp., R. microsporus, R. oryzae, and S. prolificans. Additional broad spectrum antimicrobial peptides are those sequences as set forth in SEQ ID NO:57-61 (D6, D7, 08, D9 and 010 respectively), as well as others matching the consensus sequence set forth in SEQ ID NO:62. For peptides D6-D8, there are 10 hydrophobic interactions, and for peptides D9 and D10 there are nine hydrophobic interactions. Those sequences can be comprised of all or a portion of the amino acid residues in the D or L configurations, although certain peptides specifically exemplified herein are comprised of all D amino acids. An exemplary consensus antimicrobial peptide sequence is given below and set forth in SEQ ID NO:62:
KWKSFLKTFKSX¹X²KTX²LHTX¹LKX¹ISS, wherein at positions 12, 20 and 23, independently of one another, X¹ can be a hydrophobic D or L amino acid including leucine, valine or alanine; and at positions 13 and 16, independently of one another, X² can be a basic amino acid including lysine, arginine, histidine, ornithine, diaminobutyric acid or diaminopropionic acid. Importantly, there are one or two basic (positively charged) amino acids on the nonpolar face of the helical structure of the peptide.

The antimicrobial peptides of the present invention are useful as bactericides and/or bacteriostats for modification of infectivity, killing microorganisms, or inhibiting microbial growth or function; they are useful for the treatment of infection or treatment or prevention or reduction of contamination caused by microorganisms.

Also provided are therapeutic or otherwise active compositions suitable for human, veterinary, agricultural or pharmaceutical use, comprising one or more of the antimicrobial peptides of the invention in an effective amount and a suitable pharmaceutical or agriculturally acceptable carrier. Such therapeutic compositions can be formulated and administered as known in the art, e.g., for oral, parenteral, inhalation or topical application for controlling and/or reducing infection by a wide range of microorganisms including gram-positive, gram-negative and acid-fast bacteria such as mycobacteria, and fungi. In vitro antimicrobial activity of these peptides as demonstrated herein is an accurate predictor of in vivo antimicrobial activity. A therapeutically effective amount of an antimicrobial peptide can be determined using methods well known in the art. The amount may vary depending on severity and location of infection, age and size/weight of a subject, particular target microorganism, route of administration and the like.

The present invention relates to compositions comprising one or more antimicrobial peptides of the invention in a therapeutically or microbicidally effective amount and a pharmaceutically acceptable carrier. Such compositions may further comprise a detergent, surfactant or other compound or composition (such as an amphiphilic polymer or copolymer, e.g. polyethylene glycol) to reduce peptide self-aggregation and/or improve solubility. The addition of a detergent or the like to such compositions enhances antibacterial activity and by reducing self-association can reduce toxicity. Although any suitable detergent or surfactant may be used, the presently preferred detergent is a nonionic detergent such as Tween 20 (polyoxyethylene sorbitan monolaurate) or 1% NP40 (nonyl phenoxylpolyethoxylethanol). Such antimicrobial pharmaceutical compositions can be formulated and administered, as understood in the art, with local or systemic injection, or oral or topical application. Such compositions can comprise from 0.0001% to 50% by weight of antimicrobial peptides. The compositions of the present invention can optionally comprise additional therapeutic or other compounds (including but not limited to one or more of analgesic, anti-inflammatory, antimicrobial, anticancer).

It is understood that a composition for administration, e.g. by systemic injection, contains an antimicrobial peptide in a therapeutically effective amount, or a therapeutically effective amount of an antimicrobial peptide can be conjugated to another molecule with specificity for the target cell type. The other molecule can be an antibody, ligand, receptor, or other recognition molecule. The choice of antimicrobial peptide is made with consideration of immunogenicity and toxicity for an actually or potentially infected host, effective dose of the peptide, and the sensitivity of the target microbe to the peptide, as known in the art. In other embodiments, at least one antimicrobial peptide of the present invention can be formulated for topical administration using excipients known to the art. Also, the peptide can be conjugated with a stabilizing molecule such as polyethylene glycol. Moreover, such a composition can further comprise an additional therapeutic agent, such as an antifungal, antibacterial, antinflammatory, analgesic or anticancer agent.

In an embodiment, the method of inhibiting the growth of bacteria using the peptides of the invention may further include the addition of one or more other antimicrobial agents (e.g. a conventional antibiotic) for combination or synergistic therapy. The appropriate amount of the peptide administered depends on the susceptibility of a bacterium or fungus, and is easily discerned by the ordinarily skilled artisan.

In an embodiment the invention also provides a composition that comprises the peptide, in an amount effective to kill a microorganism, and a suitable carrier. Such compositions may be used in numerous ways to combat microorganisms, for example in household or laboratory antimicrobial formulations using carriers well known in the art.

In an embodiment, the invention provides a peptide comprising SEQ ID NO:56 (D5). In an embodiment, the invention provides a peptide derived in sequence from SEQ ID NO:24, improved as to antimicrobial activity relative to the peptide of SEQ ID NO:24. In an embodiment, the invention provides a peptide selected from the group consisting of SEQ ID NO:53-61, or meeting the consensus sequence set forth in SEQ ID NO:62, and a derivative of one of the foregoing. In an embodiment, a derivative comprises a substitution of at least one amino acid residue in comparison to the D5 sequence. Peptide sequences set forth in SEQ ID NOs:1-52 are specifically excluded in the context of the present invention. The amino acids in a peptide of the present invention can be either all L-amino acids, all D-amino acids or a mixture of the two enantiomers. The peptide N-terminus can be acylated or nonacylated, or it can be substituted with another moiety known in the art to increase peptide stability, persistence or solubility, especially in the presence of biological materials. Advantageously the N-terminus is blocked, e.g. with an acetyl group or polyethylene glycol. The C-terminus can optionally comprise an amide group rather than a carboxyl group.

In an embodiment, a derivative comprises a truncation of at least one residue from an end of the peptide. The truncation of at least two residues from an end of the peptide. In an embodiment, a substitution replaces a hydrophilic residue with a hydrophobic residue, or in another embodiment, a substitution replaces a hydrophobic residue with a hydrophilic residue. In an embodiment, a substitution replaces a hydrophobic residue with a different hydrophobic residue, or in another embodiment, a substitution replaces a hydrophilic residue with a different hydrophilic residue. In an embodiment, a substitution is a different residue having a similar property, e.g., a polar side chain, a positively charged side chain, a negatively charged side chain, etc. In an embodiment, a substitution replaces an L-residue with a D-residue or a D-residue with an L-residue. In an embodiment, all residues are D-residues.

In an embodiment, the invention provides peptides or fragments thereof, wherein the fragment is at least about 14, at least about 17, at least about 20, at least about 23, at least about 24, or at least about contiguous 25 amino acids of one of SEQ ID NOs:53-62. In an embodiment, the invention provides a peptide consisting of a sequence wherein said sequence is at least about 70%, at least about 80%, at least about 90%, or at least about 95% homologous to a sequence of a peptide described herein, but is not a peptide sequence known to the art. In an embodiment, the invention provides a nucleic acid encoding a peptide described herein. A peptide of the invention is intended not to include a peptide sequence of SEQ ID NOs:1-52. It is understood that with respect to peptides of the present invention, the sequence of an antimicrobial peptide does not encompass a peptide whose sequence is known to the art as of the priority date of the present application, except as related to certain antifungal peptides, methods and compositions.

Where the peptides are used as antimicrobial agents, they can be formulated in buffered aqueous media containing a variety of salts and buffers. Examples of the salts include, but are not limited to, halides, phosphates and sulfates, e.g., sodium chloride, potassium chloride or sodium sulfate. Various buffers may be used in therapeutic compositions, such as citrate, phosphate, HEPES, Tris or the like provided that such buffers are physiologically acceptable to the subject being treated. In addition, there can be surfactants or amphiphilic polymers or other compound(s) to improve solubility, for example, provided there is no detrimental toxicity when the composition is for therapeutic use. Appropriate formulations are selected according to the administration intended: topical, mucosal, inhaled, oral or intravenous, for example.

Various excipients or other additives may be used, especially where the peptides are formulated as lyophilized powders, for subsequent use in solution. The excipients may include polyols, sugars, inert powders or other extenders.

“Therapeutically effective” as used herein, refers to an amount of formulation, composition, or reagent, optionally in a pharmaceutically acceptable carrier, that is of sufficient quantity to ameliorate the state of the patient or animal so treated. “Ameliorate” refers to a lessening of the detrimental effect of the disease state or disorder in the recipient of the therapy. In an embodiment, a peptide of the invention is administered to a subject in need of treatment.

Pharmaceutically acceptable carriers include sterile or aqueous or nonaqueous solutions, suspensions, and emulsions. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, e.g. saline and buffered media. Parenteral vehicles include sodium chloride, Ringer's dextrose, dextrose and sodium chloride, lactated Ringer's, or fixed oils. Active therapeutic ingredients can be mixed with pharmaceutically acceptable excipients which are compatible therewith such as water, saline, dextrose, glycerol and ethanol, or combinations thereof. Intravenous vehicles include fluid and nutrient replenishers, electrolyte replenishers, such as those based on Ringer's dextrose, and the like. Preservatives and other additives including but not limited to antioxidants, chelating agent, inert gases and the like may also be present. The actual dosage of the peptides, formulations or compositions containing such peptides can depend on many factors including subject size/weight, age, and health, and one of ordinary skill can use the following teachings and others known in the art describing the methods and techniques for determining clinical dosages (Spiker B., Guide to Clinical Studies and Developing Protocols, Raven Press, Ltd., New York, 1984, pp. 7-13, 54-60; Spiker B., Guide to Clinical Trials, Raven Press, Ltd., New York 1991, pp. 93-101; C. Craig. and R. Stitzel, eds., Modern Pharmacology, 2d ed., Little, Brown and Co., Boston, 1986, pp. 127-133; T. Speight, ed., Avery's Drug Treatment: Principles and Practice of Clinical Pharmacology and Therapeutics, 3d ed., Williams and Wilkins, Baltimore, 1987, pp. 50-56; R. Tallarida, R. Raffa and P. McGonigle, Principles in General Pharmacology, Springer-Verlag, new York, 1988, pp. 18-20) to determine the appropriate dosage to use. Topical application formulations can be gels, ointments, creams, salves and lotions, for example.

In an embodiment, a dosages generally in the range of about 0.001 mg/kg to about 100 mg/kg, preferably from about 0.001 mg/kg to about 1 mg/kg is administered per day to an adult in any pharmaceutically acceptable or other carrier.

In another embodiment, an antimicrobial peptide may be used as a food preservative, to treat a food product to control, reduce, or eliminate potential pathogens or contaminants, or as a disinfectant, for use in or with any product that must remain microbe-free or be within certain tolerances. In an embodiment, treatment with an antimicrobial peptide provides at least partial reduction of infection or contamination.

In an embodiment the antimicrobial peptides are incorporated or distributed within or on materials, on devices or objects (e.g. on a surface) where microbial growth or viable presence is undesirable, as a method of microbicidal or microbistatic inhibition of microbial growth by administering to the devices or objects a microbicidal or microbistatic effective amount of peptide. In an embodiment, such devices or objects include, but are not limited to, linens, cloth, plastics, latex fabrics, natural rubbers, implantable devices, surfaces, or storage containers.

An embodiment is a method of disinfecting a surface of an article, said method comprising the step of applying to said surface an effective amount of a composition comprising at least one antimicrobial peptide of the invention. In an embodiment, a disinfecting solution comprises at least one antimicrobial peptide of the invention and a acceptable carrier, and optionally another component which enhances or adds to the activity of the peptide, for example a surfactant, or another antimicrobial ingredient.

### BRIEF DESCRIPTION OF THE DRAWINGS

FIG. 1, Panel A, provides helical wheel (top)/helical net (bottom) representation of the sequences of lead compound D1 and analogs shown in Table 3. The peptides are denoted D1, D4 and D5 (SEQ ID NO:24, 55 and 56, respectively). The alanine to leucine substitutions (position 12, 20 and 23) are colored yellow. The lysine residue at position 13 and valine to lysine substitution at position 16 are denoted by blue triangles. In the helical wheel, the nonpolar face is indicated as an open arc and the polar face is shown as a solid arc. In the helical net, the amino acid residues on the non-polar face are circled. The and i→i+3 and i→i+4 potential hydrophobic interactions along the helix are shown as black bars. The numbers of hydrophobic interactions on the nonpolar face are indicated at the bottom of each helical net. The one-letter code is used for amino acid residues. FIG. 1, Panel B provides the helical wheel and helical net representations for peptides D6-D10, which have the sequences set forth in SEQ ID NOs:57-61, respectively.

FIG. 2 illustrates anti-tuberculosis activity of synthetic peptides against M. tuberculosis. Panel A: Time-kill analysis was used to determine the growth of M. tuberculosis in the presence of increasing concentrations of the peptides (data for D5 shown) for 7 days. Panel B: The data were then converted to a concentration-response format, and fit to a line. The point at which the line crossed the concentration of the initial inoculum (dashed line) was reported as the MIC. Panel C: Mean and standard error of four determinations of MIC for each of the five peptides were compared statistically. The filled (black) bars represent the peptide concentrations that resulted in 50% hemolysis. D5 was significantly more potent than the other peptides (p<0.001, ANOVA), and D4 was significantly less active (p<0.01, ANOVA).

FIG. 3 shows the correlation of peptide hydrophobicity with hemolytic activity (MHC₅₀) (Panel A), antimycobacterial activity (MIC) (Panel B) and antimicrobial specificity (therapeutic index) (Panel C) Hydrophobicity is expressed as the retention times of peptides in RP-HPLC at room temperature (Table 1). Lines are drawn through peptides D1 to D4 only, since these peptides systematically increase in hydrophobicity as shown in FIG. 1 and Table 6.

FIG. 4 provided circular dichroism (CD) spectra of peptides D1, D2, D3, D4 and D5. Panel A shows the CD spectra of peptide analogs in benign buffer (100 mM KCl, 50 mM NaH₂PO₄/Na₂HPO₄ at pH 7.0, 5° and Panel B shows the spectra in the presence of buffer-trifluoroethanol (TFE) (1:1, v/v). The relationships of peptide hydrophobicity and helicity are shown in Panel C. Hydrophobicity is expressed as the retention times of peptides in RP-HPLC at room temperature (Table 6).

FIG. 5 shows peptide self-association ability as monitored by RP-HPLC temperature profiling. In Panel A, the retention time of peptides are normalized to 5° through the expression (t_{Rt}−t_{R5}), where t_{R}^{t} is the retention time at a specific temperature of an antimicrobial peptide or control peptide C, and t_{R}⁵ is the retention time at 5°. In Panel B, the retention behavior of the peptides was normalized to that of control peptide C through the expression (t_{R}^{t}−t_{R}⁵ for peptides D1-D5)(t_{R}^{t}−t_{R}⁵ for control peptide C). The maximum change in retention time from the control peptide C defines the peptide association parameter, denoted PA. The relationship of peptide hydrophobicity and association ability is shown in panel C. Hydrophobicity is expressed as the retention times of peptides in RP-HPLC at room temperature (Table 6).

FIG. 6 provides the correlation of peptide hydrophobicity and antibacterial activity (MIC) for six clinical isolates of Pseudomonas aeruginosa. Hydrophobicity is expressed as the retention times of peptides in RP-HPLC at room temperature (93). The shaded area shows the optimal hydrophobicity zone for antimicrobial activity. The arrow denotes the optimal antimicrobial activity. The peptides denoted by L1, L2, L3 and L4 are identical in sequence to D1, D2, D3 and D4, respectively (Table 3), where L and D denote the all L form and all D form of the peptides, respectively.

FIG. 7 shows the correlation of peptide hydrophobicity and antibacterial activity (MIC) for gram-negative bacteria (Panel A) and gram-positive bacteria (Panel B). Hydrophobicity is expressed as the retention times of peptides in RP-HPLC at room temperature (Table 6). Lines are drawn through peptides D1 to D4 only, since these peptides systematically increase in hydrophobicity as shown in FIG. 1 and Table 6.

FIG. 8 illustrates correlation of peptide hydrophobicity and antifungal activity (MIC50) for Zygomycota (Panel A) and Ascomycota fungi (Panel B). Hydrophobicity is expressed as the retention times of peptides in RP-HPLC at room temperature (Table 6). Lines are drawn through peptides D1 to D4 only, since these peptides systematically increase in hydrophobicity as shown in FIG. 1 and Table 6.

FIG. 9 illustrates the hemolytic activity of peptides D1 and analogs. The concentration-response curves of peptides for lysis of human red blood cells (hRBC) are shown in Panel A. The relationship of peptide hydrophobicity and HC50 (peptide concentration that causes 50% hemolysis) is shown in Panel B. Hydrophobicity is expressed as the retention times of peptides in RP-HPLC at room temperature (Table 6). Lines are drawn through peptides D1 to D4 only, since these peptides systematically increase in hydrophobicity as shown in FIG. 1 and Table 6.

FIG. 10 illustrates a time-kill analysis to determine the grown of M. tuberculosis H37Rv in the presence of increasing concentrations of the peptide for 7 days. Diamonds, squares, triangles and circles denote 0, 01, 10 and 100 μg/mL. In the right panel, the data were then converted to a concentration-response format, and fit to a line. The point at which the line crossed the concentration of the initial inoculum (dashed line) was reported as the MIC.

FIG. 11 illustrates a time-kill analysis to determine the grown of M. tuberculosis (multidrug resistant strain vertulo) in the presence of increasing concentrations of the peptide for 7 days. Diamonds, squares, triangles and circles denote 0, 01, 10 and 100 μg/mL. In the right panel, the data were then converted to a concentration-response format, and fit to a line. The point at which the line crossed the concentration of the initial inoculum (dashed line) was reported as the MIC.

FIG. 12 illustrates the anti-tuberculosis activity of synthetic L- and D-LL-37 peptide against M. tuberculosis H37Rv (upper) and the multidrug resistant vertulo strain (lower). The left panels show time-kill analysis to determine the grown of M. tuberculosis H37Rv and vertulo strain in the presence of increasing concentrations of the peptide for 7 days. Open symbols denote L-LL-37 and closed symbols denote D-LL-37. Crosses, squares, triangles and circles denote 0, 01, 10 and 100 μg/mL. On the right, the data were converted to a concentration-response format, and fit to a line. The point at which the line crossed the concentration of the initial inoculum (dashed line) was reported as the MIC.

### DETAILED DESCRIPTION OF THE INVENTION

In general the terms and phrases used herein have their art-recognized meanings, as found in standard texts, scientific publications and contexts known to those skilled in the art. The following definitions are provided to clarify use in the context of the invention.

As used herein, the term “amino acid” refers to a natural or unnatural amino acid, whether made naturally or synthetically, including the L- or D-configuration. The term can also encompass amino acid analog compounds used in peptidomimetics or in peptoids, a modified or unusual amino acid, amino acid analog or a synthetic derivative of an amino acid, e.g. diaminobutyric acid and diaminopropionic acid and the like. In the peptide sequences, X_{L} and Xᵢ) denote the L- or D-substituting amino acids. P denotes the polar face and N denotes the non-polar face. Ac denotes N_{α}-acetyl and amide denotes C_{α}-amide.

The antimicrobial peptides of the invention are composed of amino acids linked by peptide bonds. The peptides are in general in helical conformation under hydrophobic conditions. Sequences are given from the amino terminus to the carboxyl terminus. Unless otherwise noted, the amino acids are L-amino acids. When all the amino acids are of L-configuration, the peptide is said to be an L-enantiomer. When all the amino acids are of D-configuration, the peptide is called a D-enantiomer. The α-helical peptide has a non-polar face or hydrophobic surface on one side of the molecule and a polar and positively-charged surface on the other side of the molecule; i.e., it is amphipathic. Amphipathicity of the peptide can be calculated as described herein.

The term “minimal inhibitory concentration” (MIC) refers to the lowest concentration of an antimicrobial agent (e.g., a peptide) required to prevent growth or otherwise modify a function of a microorganism under certain conditions, for example in liquid broth medium, determined using techniques well known in the art.

The term “minimal hemolytic concentration” (MHC) refers to the lowest concentration of an agent or peptide required to cause hemolysis of blood cells. MHC can be determined with red blood cells (RBC) from various species including human red blood cells (hRBC). HC_{R}) is the peptide concentration that causes 50% hemolysis of human red blood cells.

The term “therapeutic index” (TI) is the ratio of minimal hemolytic concentration (MHC) to minimal inhibitory concentration (MIC) of an antimicrobial agent. Larger values generally indicate greater antimicrobial specificity.

The term “stability” can refer to resistance to degradation, persistence in a given environment, and/or maintenance of a particular structure. For example, peptide stability can indicate resistance to proteolytic degradation, maintenance of α-helical structural conformation and/or persistence in the body or in circulation in the body or in a nonaggregated state.

The following abbreviations are used herein: A, Ala, Alanine; M, Met, Methionine; C, Cys, Cysteine; D, Asp, Aspartic Acid; E, Glu, Glutamic Acid; F, Phe, Phenylalanine; G, Gly, Glycine; H, H is, Histidine; I, Ile, Isoleucine; K, Lys, Lysine; L, Leu, Leucine; N, Asn, Asparagine; P, Pro, Proline; Q, Gln, Glutamine; R, Arg, Arginine; S, Ser, Serine; T, Thr, Threonine; V, Val, Valine; W, Trp, Tryptophan; Y, Tyr, Tyrosine; Orn, Ornithine; RP-HPLC, reversed-phase high performance liquid chromatography; MIC, minimal inhibitory concentration; MHC, minimal hemolytic concentration; CD, circular dichroism spectroscopy; TFE, trifluoroethanol; TFA, trifluoroacetic acid; RBC, red blood cells; hRBC, human red blood cells.

The term “antimicrobial activity” is the ability of a peptide of the present invention to modify a function or metabolic process of a target microorganism, for example so as to negatively affect replication, vegetative growth, toxin production, survival, viability in a quiescent state, or other attribute, especially inhibition of growth of a microorganism. In a particular embodiment, antimicrobial activity relates to the ability of a peptide of the present invention to kill at least one bacterial or fungal species. The microbe can be a gram-positive bacterium, gram-negative bacterium, acid-fast and/or mycobacterium, including but not limited to a mycobacterial species, a fungus, especially a pathogenic fungus. In an embodiment, the antimicrobial activity can be microbicidal or microbistatic.

The phrase “improved biological property” means that a test peptide exhibits less hemolytic activity and/or better antimicrobial activity, or better antimicrobial activity and/or less hemolytic activity, compared a reference peptide (e.g. V₆₈₁), when tested by the protocols described herein or other art-known protocols. In general, the improved biological property of the peptide is reflected in a therapeutic index (TI) value which is higher than that of the reference peptide.

The term “microorganism” or “microbial species” refers broadly to bacteria, fungi, viruses, and protozoa, and encompasses pathogenic bacteria, fungi, viruses, and protozoa. Bacteria can include gram-negative and gram-positive bacteria in addition to organisms classified in orders of the class Mollicutes and the like, such as species of the Mycoplasma and Acholeplasma genera, as well as others including Mycobacterium species, for example M. tuberculosis. Specific examples of potentially sensitive gram-negative bacteria include, but are not limited to, Escherichia coli, Pseudomonas aeruginosa, Salmonella, Hemophilus influenza, Neisseria, Vibrio cholerae, Vibrio parahaemolyticus and Helicobacter pylori. Examples of potentially sensitive gram-positive bacteria include, but are not limited to, Staphylococcus aureus, Staphylococcus epidermis, Streptococcus agalactiae, Group A streptococcus, Streptococcus pyogenes, Enterococcus faecalis, Group B gram positive streptococcus, Corynebacterium xerosis, and Listeria monocytogenes. Examples of potentially sensitive fungi include yeasts such as Candida albicans. Examples of potentially sensitive viruses include enveloped viruses, and measles virus, herpes simplex virus (HSV-1 and -2), herpes family members (HIV, hepatitis C, vesicular, stomatitis virus (VSV), visna virus, and cytomegalovirus (CMV). Examples of potentially sensitive protozoa include Giardia. Acid-fast bacteria include the mycobacteria, for example, Mycobacterium tuberculosis, and nocardia.

“Therapeutically effective” as used herein, refers to an amount of antimicrobial peptide, formulation, composition, or reagent in a pharmaceutically acceptable carrier or a physiologically acceptable salt of an active compound, that is of sufficient quantity and/or antimicrobial activity to ameliorate the undesirable state of the patient, animal, material, or object so treated. “Ameliorate” refers to lessening the detrimental effect of the disease state or disorder, or reducting contamination or microbial growth, in the receiver of the treatment.

The peptides of the invention have antimicrobial activity by themselves or when covalently conjugated or otherwise associated with another molecule, e.g., polyethylene glycol or a carrier protein such as bovine serum albumin, provided that the peptides are positioned such that they can come into contact with a cell or unit of the target microorganism and so that secondary structure is not negatively affected by the conjugated moiety. These peptides may be modified by methods known in the art provided that antimicrobial activity is not destroyed or substantially compromised.

The invention may be further understood by the following non-limiting examples.

### Example 1

### Derivatives of Peptide V₆₈₁ with Modified Activity

In previous studies, the 26-residue amphipathic antimicrobial peptide with polar and non-polar faces (28), Ac-KWKSFLKTFKS-AVKTVLHTALKAISS-amide (V₆₈₁, SEQ ID NO:1) was the framework to study the effects of hydrophobicity and hydrophilicity, amphipathicity and helicity via one or more amino acid substitutions in the centers of the polar and nonpolar faces of the amphipathic helix on biological activities. D-/L-amino acid substitution sites were at the center of the hydrophobic face (position 13) and at the center of the hydrophilic face (position 11) of the helix; these substitution sites were also located in the center of the overall peptide sequence. These studies demonstrated the importance of peptide self-association; disruption of α-helical structure in benign conditions by D-amino acid substitutions or substitutions of hydrophilic/charged L-amino acids on the non-polar face can dramatically alter specificity; and these substitutions can enhance antimicrobial activity, decrease toxicity and improve antimicrobial specificity while maintaining broad spectrum activity for gram-negative and gram-positive bacteria.

Five L-amino acids (Leu, Val, Ala, Ser, Lys) and Gly were selected as the substituting residues, representing a wide range of hydrophobicities (Leu>Val>Ala>Gly>Ser>Lys (26)). Leucine replaced the native valine on the non-polar face to increase peptide hydrophobicity and amphipathicity; alanine reduced peptide hydrophobicity and/or amphipathicity while maintaining high helicity; and relatively hydrophilic serine decreased the hydrophobicity and/or amphipathicity of V₆₈₁ in the non-polar face; positively-charged lysine further decreased peptide hydrophobicity and amphipathicity. In contrast, the same amino acid substitutions on the polar face would have different effects on hydrophobicity, hydrophilicity and/or amphipathicity, since the native amino acid residue is serine on the polar face of V₆₈₁. As a result, on the polar face, leucine, valine and alanine were used to increase peptide hydrophobicity and decrease the amphipathicity of V₆₈₁, while lysine was selected to increase peptide hydrophilicity and amphipathicity. Kondejewski et al. (20, 35) and Lee et al. (25) used D-amino acid substitutions to dissociate the antimicrobial activity and hemolytic activity of gramicidin S analogs. Herein, D-enantiomers of the five L-amino acid residues were also incorporated at the same positions on the non-polar/polar face of V₆₈₁ to change peptide hydrophobicity/hydrophilicity and amphipathicity and, more importantly, to disrupt peptide helical structure. Since glycine does not exhibit optical activity and has no side-chain, the Gly-substituted analog was used as a reference for diastereomeric peptide pairs.

Peptide analogs that include a single amino acid substitution in either the polar or nonpolar faces of V₆₈₁ are divided into two categories, N-peptides (nonpolar face substitutions) and P-peptides (polar face substitutions).

A control, random coil peptide (peptide C) was designed for use as a standard for temperature profiling during RP-HPLC to monitor peptide dimerization. This 18-residue peptide (Ac-ELEKGGLEGEKGGKELEK-amide, SEQ ID NO:26) exhibited negligible secondary structure, despite the strong alpha-helix inducing properties of 50% trifluoroethanol (TFE), which mimics the membrane's hydrophobic environment, and at the low temperature of 5° C. ([θ]₂₂₂=−3,950) (29).

To determine the secondary structure of peptides in different environments, circular dichroism (CD) spectra of the peptides were measured under physiologically relevant pH and ionic strength (100 mM KCl, 50 mM aq. PO₄, pH 7, benign conditions) and also in 50% TFE to mimic the hydrophobic environment of the membrane. Peptide V₆₈₁ exhibited low α-helical content in benign conditions, i.e., [θ]₂₂₂ of −12,900 compared to −27,300 in 50% TFE, an increase in α-helical content from 45% to 94%, respectively. In benign conditions, D-amino acid substituted peptides generally exhibited considerably less α-helical structure than their L-diastereomers, reflecting the helix-disrupting properties of a single D-amino acid substitution (26). On the non-polar face, the native L-Val residue was critical for maintaining α-helical structure. Substitution with less hydrophobic amino acids (L-Ala, Gly, L-Ser and L-Lys) dramatically decreased the α-helical structure (NV_{L}, [θ]₂₂₂ of −12,900 to values ranging from −1,300 to −3,450 for NS_{L}, NK_{L}, NG and NA_{L}). Even substitution with L-Ala, which has the highest α-helical propensity of all 20 amino acids (34), could not stabilize the α-helical structure, indicating the importance of hydrophobicity on the non-polar face in maintaining the α-helical structure. In contrast, substitution of L-Val with the more hydrophobic L-Leu on the non-polar face significantly increased α-helical structure ([θ]₂₂₂ for peptide NL_{L} of −20,600 compared to peptide NV_{L} of −12,900). On the non-polar face, the helical content of L-peptides in benign buffer was related to the hydrophobicity of the substituting amino acids, i.e., NL_{L}>NV_{L}>NA_{L}>NS_{L}, NK_{L}. D-Val and D-Leu substitutions on the non-polar face dramatically decreased α-helical structure in benign medium compared to their L-counterparts. However, whether L- or D-substitutions were made on the non-polar face, high helical structure could be induced by the hydrophobic environment of 50% TFE.

L-substitutions on the polar face in benign medium had different effects on α-helical structure than on the non-polar face. Leu stabilized α-helical structure on the non-polar face and destabilized α-helical structure on the polar face. Similarly, Val destabilized α-helical structure on the polar face, while Ala and Ser destabilized helical structure on the non-polar face, and Ala and Ser stabilized α-helical structure when substituted in the polar face. Taken together, even though Ala had the highest α-helical propensity of all amino acids (34), its α-helical propensity could not overcome the need for hydrophobicity on the non-polar face. Val and Leu substitutions on the polar face decreased the amphipathicity of the helix and increased hydrophobicity. The results indicated that there should be a balance of amphipathicity and hydrophobicity for greatest helical content. As for substitutions on the non-polar face, D-amino acid substitutions on the polar face were destabilizing to α-helical structure in benign medium although highly helical structure could be induced in 50% TFE. Non-polar face substitutions exhibited a greater range of molar ellipticity values in benign conditions than polar face analogs, demonstrating that the residues on the non-polar face of the helix were more important for secondary structure than those on the polar face. Gly was destabilizing to α-helical structure whether on the non-polar or polar face due to its low α-helical propensity (34).

Enantiomeric peptides of V₆₈₁ and analogs NK_{L} and NA_{D} were prepared. Peptides V₆₈₁ and NK_{L} contain all L-amino acids and D-V₆₈₁ and D-NK_{D} contain all D-amino acids. In the case of NA_{D} and D-NA_{L}, position 13 is D-alanine and L-alanine, respectively (Table 1). Thus, D-V₆₈₁, D-NK_{D} and D-NA_{L} are opposite in stereochemistry to the corresponding L-peptides, V₆₈₁, NK_{L} and NA_{D}, respectively. Peptide C, a random coil, was the standard peptide for temperature profiling during RP-HPLC to monitor peptide dimerization (53, 19, 29).

CD spectra of the peptide analogs were measured under benign conditions (100 mM KCl, 50 mM KH₂PO₄/K₂HPO₄, pH 7.4, referred to as KP buffer) and in 50% trifluoroethanol (TFE), which mimics the hydrophobic membrane environment. Parent peptide V₆₈₁ was only partially helical in KP buffer; peptides NK_{L} and NA_{D} exhibited negligible secondary structure in KP buffer due to disruption of the non-polar face of the helix by introducing a hydrophilic L-lysine residue into peptide NK_{L} or a helix-disruptive D-alanine residue into peptide NA_{D}. In the presence of 50% TFE, all three L-peptides were fully folded α-helical structures with similar ellipticities and helicity. The D-peptides showed spectra that were exact mirror images compared to their L-enantiomers, with ellipticities equivalent but of opposite sign both in benign KP buffer and in 50% TFE.

Temperature profiling during RP-HPLC is used to determine the self-association ability which occurs through interaction of the non-polar faces of these amphipathic α-helices. Using model amphipathic α-helical peptides with all 20 amino acid substitutions in the center of the non-polar face, we showed previously that the model amphipathic peptides were maximally induced into an α-helical structure in 40% TFE and that the stability of the α-helix during temperature denaturation was dependent on the substitution (26). Temperature denaturation studies were carried out in a hydrophobic environment to study association and monitored by circular dichroism spectroscopy. The hydrophobic environment of a reversed-phase column (hydrophobic stationary phase and the hydrophobic organic solvent in the mobile phase) induced α-helical structure in a similar manner to TFE. At 5° C. in hydrophobic medium, 50% TFE induced full α-helical structure of V₆₈₁. The helical content of V₆₈₁ decreased with increasing temperature, but even at 80° C. V₆₈₁ remained significantly α-helical. V₆₈₁ has a transition temperature Tₘ of 79.3° C., where Tₘ is defined as the temperature when 50% of α-helical structure is denatured compared with the fully folded conformation of the peptide in 50% TFE at 5° C. During temperature profiling in RP-HPLC, the peptides are fully helical at low temperatures such as 5° C. and can remain in the α-helical conformation at 80° C. in solution during partitioning in RP-HPLC. In addition, due to their hydrophobic preferred binding domains, the peptides remain α-helical when bound to the hydrophobic matrix. Overall, V₆₈₁ is a very stable α-helical peptide in hydrophobic environments.

Formation of a hydrophobic binding domain due to peptide secondary structure can affect amphipathic α-helical peptide interactions with reversed-phase matrices (26, 36-39). Zhou et al. (39) demonstrated that, because of this preferred binding domain, peptides are more retentive than non-amphipathic peptides of the same amino acid composition. In addition, the hydrophobic chromatography conditions characteristic of RP-HPLC induce and stabilize helical structure in potentially helical polypeptides (39-41) as does TFE. The substitution site at position 13 in the center of the nonpolar face of the helix maximized the effect on the intimate interaction of the substituting side-chain with the reversed-phase stationary phase. Differences in effective hydrophobicity are monitored via differences in RP-HPLC retention time.

Retention time data at 5° C., the maximal retention times and retention times at 80° C. during the temperature profiling for the substituted peptides were collected. Temperatures of 5° C. and 80° C. were the lower and upper temperature limits of temperature profiling in RP-HPLC, representing dimerization of the peptides at 5° C. and the monomerization of peptides at 80° C. due to dimer dissociation. The maximal retention time represents the threshold point where a dimeric peptide dissociates to monomers. Peptides with more hydrophobic substitutions (L- or D-amino acid substitutions) in the nonpolar face were more retained during RP-HPLC, i.e., substituted peptides were eluted in the order Lys, Gly, Ser, Ala, Val and Leu. In addition, the L-analogs on the non-polar face were always retained longer than the D-diastereomers. Because the preferred binding domain of amphipathic helices is actually the non-polar face of the helix, D-peptides had a smaller preferred binding domain compared with L-diastereomers due to the helix disruptive ability of D-amino acids, resulting in shorter retention times with RP-HPLC. In contrast, the elution order of peptides with substitutions on the polar face was not correlated with amino acid side-chain hydrophobicity, e.g., PA_{L} and PS_{L} were more retained than PV_{L}; and PS_{D} was the most retained peptide of the polar face D-amino acid substituted analogs. Peptides PL_{L} and PA_{L} substituted on the polar face (replacement of L-Ser by L-Leu or L-Ala), had increased overall hydrophobicity, resulting in higher retention times as compared with V₆₈₁.

Although L-Val is much more hydrophobic than L-Ser, peptide PV_{L} was less retained than the native peptide V₆₈₁ (L-Ser at position 11 of the polar face) perhaps due to the helix-disrupting characteristics of the β-branched Val residue. In contrast, at 80° C., PV_{L} was more retained than PS_{L}. With the unfolding of helical structure at high temperature, the side-chain hydrophobicity of the substituting amino acid in the peptide is more important in overall hydrophobicity. As for the non-polar face substituted peptides, peptides with D-amino acids substituted into the polar face were dramatically less retained than their L-diastereomers. Due to the effect of the preferred binding domain, peptides with non-polar face substitutions had a greater retention time range than those with polar face substitutions.

The ability of the D-peptides to self-associate was determined by RP-HPLC temperature profiling (5° C. to 80° C.). L- and D-peptide enantiomers were equivalent over this range (each pair of peptides is identical in sequence and adopts identical conformations on interacting with the reversed-phase matrix).

RP-HPLC retention behavior has been used to estimate overall peptide hydrophobicity (53,26). The hydrophobicity was in the order V₆₈₁/D-V₆₈₁>NA_{D}/D-NA_{L}>NK_{L}/D-NK_{D}, consistent with the decreasing hydrophobicity of the substitutions at position 13 (Val in V₆₈₁>Ala in NA>Lys in NK) (54). Increased retention as temperature increases up to ˜30° C., followed by decreased retention time above about 30° C. is characteristic of a self-associating peptide (53, 29, 19). The peptide self-association parameter, P_{A}, represents the maximum change in peptide retention time relative to the random coil peptide C. Because peptide C is a monomeric random coil peptide in aqueous and hydrophobic media, its retention behavior over the temperature range 5° C. to 80° C. represents only general temperature effects on peptide retention behavior, i.e., a linear decrease in peptide retention time with increasing temperature due to greater solute diffusivity and enhanced mass transfer between the stationary and mobile phases at higher temperatures (55). After normalization, the retention behavior of the peptides represents only peptide self-association ability. The higher the P_{A} value, the greater the self-association ability. Peptide self-association is positively correlated with peptide hydrophobicity. Peptide retention times at 80° C. were dramatically lower than at 5° C., in part due to unraveling of the α-helix that occurs with increasing temperature, and loss of the non-polar face of the amphipathic α-helical peptides.

Elution times during RP-HPLC reflect relative hydrophobicity of peptide analogs (26,31). To enhance differences in hydrophobicity, the retention time data can be normalized relative to a reference peptide at 5° C. and 80° C. Hydrophobicity relative to the native peptide V₆₈₁ or other reference indicates an increase or decrease of the apparent peptide hydrophobicity with the different amino acid substitutions on the polar or non-polar face. For non-polar face substituted peptides, there was a wide range of peptide hydrophobicities (L-Leu>L-Val>L-Ala>L-Ser>Gly>L-Lys) at both 5° C. and 80° C. The relative hydrophobicities of D-peptides was always less than their L-diastereomers because the helix-disrupting characteristics of D-amino acids affect the preferred binding domain of the helices. On both non-polar and polar faces, peptides exhibited a greater retention time range at 80° C. than at 5° C., also indicating that, due to the unfolding of the helical structures at 80° C., the side-chain hydrophobicity of the substituted amino acids has a greater influence on the overall hydrophobicity of the peptide analogs.

The hydrophobicity/hydrophilicity effects of substitutions on the non-polar face relative to the native peptide V₆₈₁ were large. For example, NV_{L} to NA_{L}, to NS_{L}, and to NK_{L} resulted in decreases in hydrophobicity of −4.45, −8.21 and −12.61 min at 80° C., respectively. In fact, the same substitutions, i.e., PV_{L} to PA_{L}, to PS_{L}, and to PK_{L}, resulted in overall hydrophobicity changes of the peptide by +0.45, −0.35 and −2.29 min at 80° C., respectively. This indicates that the polar face substitutions affected overall hydrophobicity of the peptide in a minor way relative to substitutions on the non-polar face. In fact, the effect was of 10 times less for Ala, >20 times less for Ser and >5 times less for Lys.

The RP-HPLC temperature profiling technique has been applied to various molecules, including cyclic 13-sheet peptides (30), monomeric α-helices and α-helices that dimerize (29), and α-helices that dimerize to form coiled-coils (42). Although peptides are eluted from a reversed-phase column mainly by an adsorption/desorption mechanism (43), even a peptide strongly bound to a hydrophobic stationary phase partitions between the matrix and the mobile phase when the acetonitrile content becomes high enough during gradient elution. This proposed mechanism for temperature profiling of α-helical peptides in RP-HPLC is based on four assumptions: at low temperature, just as an amphipathic α-helical peptide is able to dimerize in aqueous solution (through its hydrophobic, nonpolar face), it dimerizes in solution during partitioning in reversed-phase chromatography; at higher temperatures, the monomer-dimer equilibrium favors the monomer as the dimer is disrupted; at sufficiently high temperatures, only monomer is present in solution; and peptide is always bound in its monomeric helical form to the hydrophobic stationary phase, i.e., the dimer can only be present in solution and disruption of the dimer is required for rebinding to the RP-HPLC matrix.

Antimicrobial peptides must be amphiphilic for antimicrobial activity, because the positively-charged polar face helps the molecules reach the biomembrane through electrostatic interaction with the negatively-charged head groups of phospholipids, and then the nonpolar face of the peptides allows insertion into the membrane through hydrophobic interactions, causing increased permeability and loss of barrier function of target cells (6,7). Peptide self-association in aqueous solution is an important parameter; if the self-association ability of a peptide in aqueous media is too strong (dimers bury the non-polar face), it decreases the ability to dissociate and penetrate into the biomembrane and to kill target cells.

Temperature profiling of L-/D-amino acid substituted peptides during RP-HPLC from 5° C. and 80° C. confirmed that dimerization is temperature-dependent. At low temperature RP-HPLC partitioning, peptides exist in a dimer-monomer equilibrium, with the dimeric unbound state favored and dissociation required for rebinding; thus, the retention times are relatively low. With the increase of temperature, equilibrium is shifted toward the monomeric form in solution due to the disruption of the dimer. The higher solution concentration of monomer during partitioning increases the on-rate for the bound state, and the retention time increases. Increased temperature also influences retention time because of lower mobile phase viscosity and increase in mass transfer between stationary and mobile phases, leading to a linear decrease in retention time with increasing temperature. Conversely, for dimerized peptides, maximum retention time results at the temperature where dimers are disrupted and converted to monomers. Above this critical temperature, retention time decreases with increasing temperature. In addition, the temperature-induced conformational changes, monitored by CD, may also have an impact due to the destabilization of peptide α-helical structure and loss of preferred binding domain at higher temperatures.

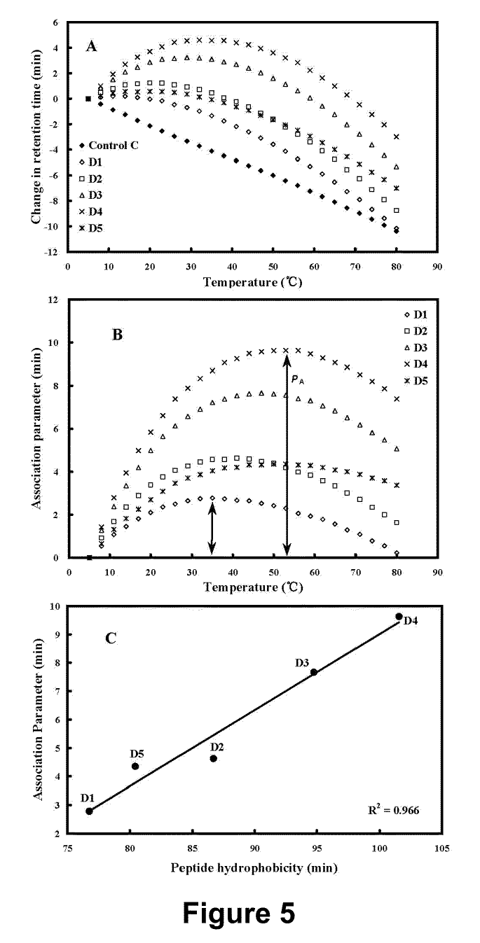

Peptide variants showed dramatic varying dimerization ability in solution. The maximal values of the change of retention times ((t_{R}^{t}−t_{R}⁵ for peptide)-(t_{R}^{t}−t_{R}⁵ for C)) were defined as the peptide association parameter (P_{A}) to quantify the association ability of peptide analogs in solution. Peptides with higher relative hydrophobicity generally showed stronger self-association ability in solution. The P_{A} values of the peptide with non-polar face substitutions were of the same order as their relative hydrophobicity, indicating that the hydrophobicity on the hydrophobic face of the amphipathic helix was essential during dimerization, since the dimers are formed by the binding together of the non-polar faces of two amphipathic molecules. In contrast, the different relationship between P_{A} and the relative hydrophobicity of the peptides with polar face substitutions demonstrated that the hydrophobicity on the polar face of the helices is less important in peptide association. Generally, the P_{A} values of L-peptides were significantly greater than those of their D-diastereomers, indicating the importance of helical structure during dimerization, peptides with polar face substitutions usually had greater P_{A} values than the corresponding peptide analogs with the same amino acid substitutions on the non-polar face; polar face substitutions have little effect on the preferred dimerization domain, whereas non-polar face substitutions dramatically affect the hydrophobicity and dimerization ability. See FIG. 5 for results with peptides D1-D5 (SEQ ID NO:24 and 53-56).

Amphipathicity of the L-amino acid substituted peptides is determined by the calculation of hydrophobic moment (32) using the software package Jemboss version 1.2.1 (33), modified to include the hydrophobicity scale determined as described below. Peptide amphipathicity, for the non-polar face substitutions, was directly correlated with side-chain hydrophobicity of the substituted amino acid residue, i.e., the more hydrophobic the residue the higher the amphipathicity (values of 6.70 and 5.60 for NL_{L} and NK_{L}, respectively); in contrast, on the polar face, peptide amphipathicity was inversely correlated with side-chain hydrophobicity of the substituted amino acid residue, i.e., the more hydrophobic the residue, the lower the amphipathicity (compare PK_{L} and PL_{L} with amphipathicity values of 6.62 and 5.45, respectively.

The native sequence (SEQ ID NO:1), V₆₈₁ was very amphipathic with a value of 6.35. To place this value in perspective, the sequence of V₆₈₁ was shuffled to obtain an amphipathic value of 0.96 (KHAVIKWSIKSSVKFKISTAFKATTI, SEQ ID NO: 41) or a maximum value of 8.10 for the sequence of HWSKLLKSFTKALKKFAKAITSVVST (SEQ ID NO:42). The range of amphipathicity values achieved by single substitutions on the polar and non-polar faces varied from a low of 5.45 for PL_{L} to a high of 6.70 for NL_{L}. Even though single substitutions changed the amphipathicity, all the analogs remained very amphipathic, e.g., even with a lysine substitution on the non-polar face, NK_{L} has a value of 5.60.

Many models have been proposed for the mechanism of action of antimicrobial peptides, including the “barrel-stave” mechanism and the “carpet” model (44). The “barrel-stave” mechanism describes the formation of transmembrane channels/pores by bundles of amphipathic α-helices as their hydrophobic surfaces interact with the lipid core of the membrane and the hydrophilic surfaces point inward, producing an aqueous pore (45); in contrast, the “carpet” model was proposed to describe the mechanism of action of dermaseptin S (46), with contact of antimicrobial peptides with the phospholipid head group throughout the entire process of membrane permeation, which occurs only if there is a high local concentration of membrane-bound peptide. The major difference between the two mechanisms is, in the carpet model, peptides lie at the interface with their hydrophobic surface interacting with the hydrophobic component of the lipids but are not in the hydrophobic core of the membrane, and neither do they assemble the aqueous pore with their hydrophilic faces. A NMR study has shown that the cyclic (3-sheet peptide analog of gramicidin S lays in the interface region parallel with the membrane where its hydrophobic surface interacts with the hydrophobic fatty acyl chains and the positively charged residues can still interact with the negatively charged head groups of the phospholipids (47).

Whichever the mechanism, the peptide molecule must be attracted to the membrane and then inserted into the bilayer. Peptides with less self-association in aqueous media more easily penetrate the lipid membrane. Peptides with higher relative hydrophobicity on their non-polar faces created higher amphipathicity and generally showed stronger self-associating ability in solution; while for peptides with polar face substitutions, increasing hydrophobicity lowers amphipathicity, yet the peptides still strongly self-associate, which indicates that peptide amphipathicity plays a less important role in peptide self-association when changes in amphipathicity are created on the polar face. In addition, self-association is correlated with the secondary structure of peptides, i.e., disrupting the peptide helical structure by replacing the L-amino acid with its D-amino acid counterpart decreases the P_{A} values.

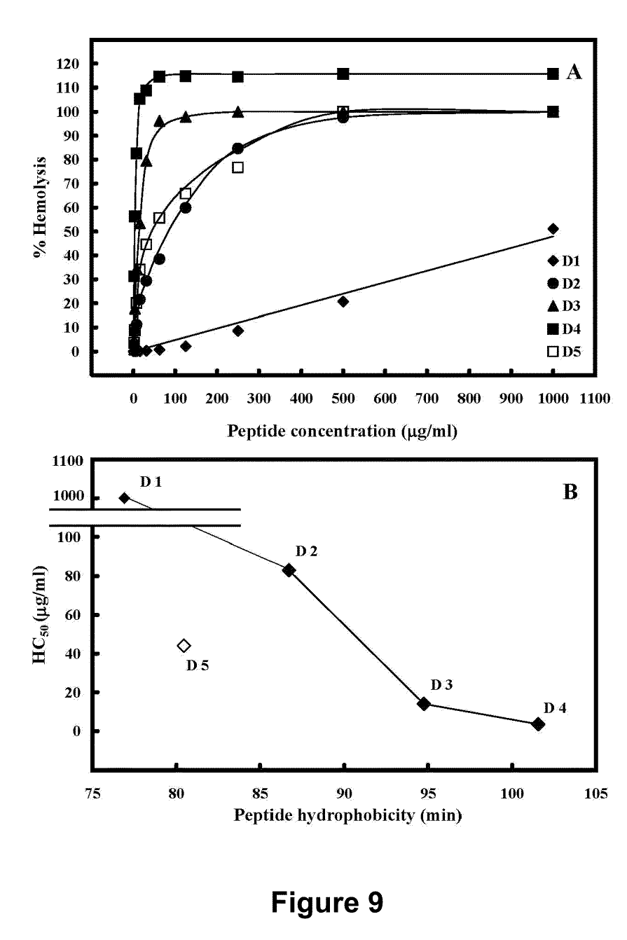

The hemolytic activity of the peptides for human erythrocytes reflects peptide toxicity toward higher eukaryotic cells. As mentioned before, the native peptide V₆₈₁ (SEQ ID NO:1; NV_{L} or PS_{L}) had strong hemolytic activity, with a minimal hemolytic concentration (MHC value) of 15.6 μg/ml. In previous work by altering hydrophobicity, amphipathicity and stability, the hemolytic activity of the variants was decreased to no detectable activity, a >32 fold decrease for NK_{L}. In the studies described herein, the hemolytic activity was further decreased with further manipulations of peptide primary structure; see FIG. 9 and Table 7.

For the non-polar face substituted peptides, hemolytic activity was correlated with the side-chain hydrophobicity of the substituting amino acid residue, i.e., the more hydrophobic the substituting amino acid, the more hemolytic the peptide, consistent with our previous study on the β-sheet antimicrobial peptide gramicidin S (39). For example, the MHC of peptide NL_{L} was 7.8 μg/ml; in contrast, the MHC was decreased, parallel with the reduction of hydrophobicity, to an undetectable level for peptide NK_{L}. Peptide hydrophobicity and amphipathicity on the non-polar face were also correlated with peptide self-associating ability, thus peptides with less self-association in benign conditions also exhibited less hemolytic activity against eukaryotic cells. In contrast, for polar face substituted peptides, the relationships between self-association, hydrophobicity/amphipathicity and hemolytic activity were less clear. Of course, the hydrophobic non-polar face remained very similar when L-substitutions were made on the polar face; thus, dimerization and hydrophobicity of the non-polar face would be less affected and hemolytic activity would remain relatively strong.

In addition to hydrophobicity/amphipathicity, peptide helicity seemed to have an additional effect on hemolytic activity. In general, on both the non-polar and polar faces, D-amino acid substituted peptides were less hemolytic than their L-diastereomers. For example, NA_{L} had a MHC value of 31.2 μg/ml compared to NA_{D} with a value of 250 μg/ml, an 8-fold decrease in hemolytic activity. Similarly, PV_{L had a MHC value of} 7.8 μg/ml compared to PV_{D} with a value of 125 μg/ml, a 16-fold decrease in hemolytic activity. This phenomenon generally correlated with peptide self-associating ability, since D-diastereomeric analogs exhibited weaker self-associating ability than L-analogs. Additionally, D-substitutions disrupt helicity which, in turn, disrupts hydrophobicity of the non-polar face. This result was also consistent with the data of Shai and coworkers (23,24), who demonstrated that, through multiple D-amino acid substitutions, the helicity of peptides is substantially reduced leading to decreased hemolytic activity. Thus, peptide structure is important in the cytotoxicity towards mammalian cells although these disturbed helices can still maintain antibacterial activity.

Peptide variants with non-polar face substitutions exhibited a greater range of hemolytic activity (7.8 μg/ml to not detectable) than the polar face substitutions (4 to 125 μg/ml), again indicating that the non-polar face of the helix may play a more essential role during the interaction with the biomembrane of normal cells. As expected, the peptides with the polar face substitutions showed stronger hemolytic activity than the peptides with the same amino acid substitutions on the non-polar face, which may be attributed to the different magnitude of the hydrophobicity change by the same amino acid substitutions on different sides of the amphipathic helix. Interestingly, in previous studies, all polar face substituted peptides except PL_{D}, PV_{D} and PK_{D} showed stronger hemolysis of erythrocytes than V₆₈₁; in contrast, on the non-polar face, only peptides NL_{D} and NL_{L} were more hemolytic than V₆₈₁.

The antimicrobial activity was determined for peptides with either non-polar face or polar face amino acid substitutions against a range of gram-negative microorganisms. The geometric mean MIC values from 6 microbial strains were calculated to provide an overall evaluation of antimicrobial activity against gram-negative bacteria. Many peptide analogs showed considerable improvement in antimicrobial activity against gram-negative bacteria over the native peptide V₆₈₁, e.g., peptides NK_{L} and PK_{D} exhibited 2.8-fold and 3.4-fold improvement on the average MIC value compared to V₆₈₁, respectively (geometric mean comparison). Generally, the peptide analogs have high activity against bacterial strains of E. coli (UB 1005 wt and DC2 abs), S. typhimurium C610 abs and P. aeruginosa H187 wt.

For gram-negative bacteria, disruption of peptide helicity outweighed other factors in increasing antimicrobial activity; i.e., in most cases, the peptides with D-amino acid substitutions showed better antimicrobial activity than L-diastereomers. See WO 2006/065977. The exceptions were peptides NS_{D} and NK_{D}, wherein the low activity of peptides NS_{D} and NK_{D} was possibly due to the combined effects of the destabilization of the helix, decreased hydrophobicity on the non-polar face and the disruption of amphipathicity, highlighting the importance of a certain magnitude of hydrophobicity and amphipathicity on the non-polar face of the helix for biological activity, i.e., perhaps there is a combined threshold of helicity and hydrophobicity/amphipathicity required for biological activity of α-helical antimicrobial peptides. In this study, peptide self-associating ability (relative hydrophobicity) seemed to have no general relationship to MIC; however, interestingly, for peptides with L-hydrophobic amino acid substitutions (Leu, Val and Ala) in the polar and non-polar faces, the less hydrophobic the substituting amino acid, the more active the peptide against gram-negative bacteria.

Antimicrobial activity of certain peptides against gram-positive microorganisms was also tested; see WO 2006/065977. By introducing D-/L-amino acid substitutions, the antimicrobial activity of peptide V₆₈₁ against gram-positive bacteria was improved by as much as 2.7-fold (geometric mean MIC values for V₆₈₁ were 6.3 μg/ml compared to 2.3 μg/ml for PS_{D}). Compared with peptide V₆₈₁, most of the peptide analogs with increased antimicrobial activity against gram-positive microorganisms were D-amino acid substituted peptides (6 D-peptides versus 1 L-peptide). Peptides with polar face substitutions showed an overall greater improvement in MIC than those with non-polar face substitutions. In general, increasing the hydrophobicity of the native peptide V₆₈₁ by amino acid substitutions at either the polar or the non-polar face decreased antimicrobial activity against gram-positive bacteria, e.g., peptides NL_{L}, PL_{L}, PV_{L} and PA_{L}. Amino acid substitutions of D-Ser and D-Lys on the non-polar face significantly weakened the activity, in a similar manner to the anti-gram-negative activity, indicating again the importance of maintaining a certain magnitude of helicity, hydrophobicity/amphipathicity on the non-polar face of the helix for Gram-positive antimicrobial activity.

Therapeutic index is a widely employed parameter to represent the specificity of antimicrobial reagents. It is calculated by the ratio of MHC (hemolytic activity) and MIC (antimicrobial activity); thus, larger values in therapeutic index indicate greater antimicrobial specificity. Peptide V₆₈₁ exhibits good antimicrobial activity but strong hemolytic activity; hence, its therapeutic index is low (1.8 and 2.5 for gram-negative and gram-positive bacteria, respectively) and comparable to general toxins like melittin. By altering peptide hydrophobicity/hydrophilicity, amphipathicity and helicity, the therapeutic index of peptide V₆₈₁ against gram-negative and gram-positive bacteria could be increased.

In prior work, peptides with improved therapeutic indices exhibited less stable helical structure in benign medium (either the D-amino acid substituted peptides or the hydrophilic amino acid substituted peptides on the non-polar face). The peptide with the best therapeutic index among all the analogs was NK_{L} (90-fold improvement compared with V₆₈₁ against Gram-negative bacteria); whereas peptide NA_{D} showed broad specificity against all gram-negative and gram-positive microorganisms tested (42-fold improvement in therapeutic index against gram-negative bacteria and a 23-fold improvement against gram-positive bacteria). The hemolytic activity of these two peptides was extremely weak; in addition, peptides NK_{L} and NA_{D} exhibited improved antimicrobial activity compared to peptide V₆₈₁ against gram-negative bacteria and identical antimicrobial activity against gram-positive bacteria.

Pseudomonas aeruginosa strains used in this study are a diverse group of clinical isolates from different geographic locations. Antibiotic susceptibility tests show that these Pseudomonas aeruginosa strains share similar susceptibility to most antibiotics except that there is about a 64-fold difference for the range of ciprofloxacin susceptibility. In general, the antimicrobial activity of L- and D-enantiomers against Pseudomonas aeruginosa varied within 4-fold. D-peptides disclosed in WO 2006/065977 generally exhibited slightly better antimicrobial activity than their L-enantiomers.

While the “barrel-stave” and the “carpet” mechanisms are the two main models used to explain the mechanism of action of antimicrobial peptides, neither fully accounts for the data disclosed in WO 2006/065977. For example, hemolytic activity is correlated to the peptide hydrophobicity and amphipathicity on the non-polar face, which may be consistent with the “barrel-stave” mechanism, i.e., peptides interact with the hydrophobic core of the membrane by their non-polar face to form pores/channels. In contrast, the antimicrobial activity is not correlated with peptide hydrophobicity/amphipathicity, suggesting that the “barrel-stave” mechanism is not sufficient to account for the antimicrobial action. Thus, the “carpet” mechanism may best explain the interaction between the peptides and the bacterial membrane. Based on those observations, it is believed both mechanisms contribute to the properties of peptides, i.e., the mechanism depends upon the difference in membrane composition between prokaryotic and eukaryotic cells. If the peptides form pores/channels in the hydrophobic core of the eukaryotic bilayer, they cause the hemolysis of human red blood cells, and the peptides lyse prokaryotic cells in a detergent-like mechanism as described in the “carpet” mechanism.

The extent of interaction between peptide and biomembrane is believed to depend on the composition of lipid bilayer. For example, Liu, et al. (48-50) utilized a polyleucine-based α-helical transmembrane peptide to demonstrate that the peptide reduced the phase transition temperature to a greater extent in phosphatidylethanolamine (PE) bilayers than in phosphatidylcholine (PC) or phosphatidylglycerol (PG) bilayers, indicating a greater disruption of PE organization. The zwitterionic PE is the major lipid component in prokaryotic cell membranes and PC is the major lipid component in eukaryotic cell membranes (51,52). In addition, although PE also exists in eukaryotic membranes, due to the asymmetry in lipid distribution, PE is mainly found in the inner leaflet of the bilayer while PC is mainly found in the outer leaflet of the eukaryotic bilayer. Without wishing to be bound by any particular theory, we have concluded that the antimicrobial specificity of the antimicrobial α-helical peptides results from composition differences of the lipid bilayer between eukaryotic and bacterial cells.

In support of this conclusion, two examples were selected. The results for peptide NK_{L} can be explained using the combined model. For example, if hemolysis of eukaryotic cells requires insertion of the peptide into the hydrophobic core of the membrane, which depends on the composition of the bilayer, and interaction of the non-polar face of the amphipathic α-helix with the hydrophobic lipid environment, it seems reasonable that disruption of the hydrophobic surface with the Lys substitution (NK_{L}) would both disrupt dimerization of the peptide and its interaction with the hydrophobic lipid. Thus, the peptide is unable to penetrate the hydrophobic core of the membrane and unable to cause hemolysis. On the other hand, if the mechanism for prokaryotic cells allows the interaction of monomeric peptides with the phospholipid headgroups in the interface region, then no insertion into the hydrophobic core of the membrane is required for antimicrobial activity.

The biological activities of certain D-enantiomeric peptides are consistent with that model; each enantiomeric peptide pair has the same activities against prokaryotic and eukaryotic cell membranes, supporting the prediction that the sole target for these antimicrobial peptides is the cell membrane. This predicts that hemolysis requires the peptides to be inserted into the hydrophobic core of the membrane, perpendicular to the membrane surface, and interaction of the non-polar face of the amphipathic α-helix with the hydrophobic lipid core of the bilayer. The peptide may thus form transmembrane channels/pores with the hydrophilic surfaces pointing inward, producing an aqueous pore (“barrel-stave” mechanism). In contrast, antimicrobial activity in prokaryotic cells, while maintaining specificity, requires the peptide to lie at the membrane interface parallel with the membrane surface and interaction of the non-polar face of the amphipathic α-helix with the hydrophobic component of the lipid and interaction of the positively charged residues with the negatively charged head groups of the phospholipid (“carpet” mechanism). What dictates the two different modes of interaction is the difference in lipid composition of prokaryotic and eukaryotic membranes: this mode of interaction of antimicrobial peptides which combines the above two mechanisms is termed the “membrane discrimination mechanism”.

This model explains why peptide NK_{L} and D-NK_{D} are relatively non-hemolytic but possess significant antimicrobial activity compared to the native sequence V₆₈₁ or D-V₆₈₁. Thus, the single substitution of Lys for Val at position 13 (NK_{L} and D-NK_{D}) in the center of the non-polar face disrupts the hydrophobic surface due to the presence of the positive charge, preventing the peptide from penetrating the bilayer as a transmembrane helix in eukaryotic cells. The peptide is then excluded from the bilayer and, hence, is non-hemolytic. In prokaryotic cells, the peptide is also excluded from penetrating the bilayer as a transmembrane helix, but this is not required for excellent antimicrobial activity. Instead, the peptide can enter the interface region of the bilayer where disruption of the peptide hydrophobic surface by Lys can be tolerated and antimicrobial activity maintained.

In contrast, the observation that the antimicrobial activity of peptide NL_{L} (with Leu at the substitution site) was weaker than that of NK_{L}, while its hemolytic activity was stronger (MIC values of 12.7 μg/ml for NL_{L} versus 3.1 μg/ml for NK_{L} against Gram-negative bacteria; hemolytic activity of 7.8 μg/ml for NL_{L} versus no detectable hemolytic activity for NK_{L}) can also be explained by the combined model. Thus, peptide NL_{L} has a fully accessible non-polar face required for insertion into the bilayer and for interaction with the hydrophobic core of the membrane to form pores/channels (“barrel-stave” mechanism), while the hemolytic activity of peptide NL_{L} is dramatically stronger than peptide NK_{L}. Due to the stronger tendency of peptide NL_{L} to be inserted into the hydrophobic core of the membrane than peptide NK_{L}, peptide NL_{L} actually interacts less with the water/lipid interface of the bacterial membrane; hence, the antimicrobial activity is 4-fold weaker than the peptide NK_{L} against Gram-negative bacteria. This supports the view that the “carpet” mechanism is essential for strong antimicrobial activity and if there is a preference by the peptide for penetration into the hydrophobic core of the bilayer, the antimicrobial activity can decrease.

The relatively strong tendency of a peptide to self-associate in solution generally correlates with relatively weak antimicrobial activity and strong hemolytic activity. Strong hemolytic activity generally correlates with high hydrophobicity, high amphipathicity and high helicity. In most cases, the D-amino acid substituted peptides exhibited enhanced antimicrobial activity compared with L-peptide counterparts. The therapeutic index of V₆₈₁ was improved 90-fold and 23-fold against gram-negative and gram-positive bacteria, respectively. Other substitutions such as ornithine, arginine, histidine or other positively charged residues such as diaminobutyric acid or diaminopropionic acid at these sites improve the antimicrobial activity of the peptides, as disclosed herein. Similar substitutions at position 16 or 17 of D1 yield peptides with enhanced biological activity. Based on the present teachings, the ordinarily artisan can design antimicrobial peptides with enhanced activities by replacing the central hydrophobic or hydrophilic amino acid residue on the nonpolar or the polar face of an amphipathic molecule with a series of selected D-/L-amino acids.

Significant features of two specific antimicrobial peptides generated from this study in structural terms are as follows. In the case of NK_{L}, a positively-charged residue, lysine, was introduced in the center of the hydrophobic face. This substitution disrupts alpha-helical structure in benign medium, decreases dimerization, decreases toxicity to normal cells as measured by hemolytic activity, enhances antimicrobial activity and provides a 90-fold increase in the therapeutic index compared with the starting sequence against Gram-negative bacteria (substitution of starting material having Val 13 with a change to Lys 13). The therapeutic index is the ratio of hemolytic activity/antimicrobial activity. This same peptide has a 17-fold increase in the therapeutic index for Gram-positive bacteria.

In the case of NA_{D}, a D-Ala residue is introduced into the center of the hydrophobic face. This disrupts alpha-helical structure, decreases dimerization, decreases toxicity to normal cells as measured by hemolytic activity, enhances antimicrobial activity and provides a 42-fold increase in the therapeutic index compared to the starting sequence against Gram-negative bacteria (substitution is Val 13 to D-Ala 13). This same peptide has a 23-fold increase in the therapeutic index for Gram-positive bacteria.

Alpha-helical antimicrobial peptides are amphipathic; if the self-association ability of a peptide (forming dimers by interaction of the two non-polar faces of two molecules) is too strong in aqueous media, the ability of the peptide monomers to dissociate and pass through the microbial cell wall to penetrate the membrane to kill target cells is decreased. It was demonstrated using the D-enantiomeric peptides that disruption of dimerization generates specificity between eukaryotic and prokaryotic cells. The P_{A} values of peptides derived from their temperature profiling data reflect the ability of the amphipathic α-helices to associate/dimerize. Clearly, V₆₈₁ and D-V₆₈₁, due to their uniform non-polar faces, show the greatest ability to dimerize in aqueous solution and lowest specificity (strongest hemolytic ability), consistent with the view that a peptide with a fully accessible non-polar face tends to form pores/channels in the membranes of eukaryotic cells. In the case of NA_{D} and D-NA_{L}, the introduction of D-Ala and L-Ala into all-L- and all-D-amino acid peptides, respectively, disrupts α-helical structure and, thus, lowers dimerization ability and improves specificity. The introduction of Lys into non-polar position 13 of NK_{L} and D-NK_{D} lowers this dimerization ability further and improves specificity. Thus, decreased dimerization, as exemplified by its P_{A} value, is an excellent measure of the peptide's nonhemolytic ability and maintenance of sufficient hydrophobicity of the non-polar face to ensure antimicrobial activity. D-enantiomeric peptides exhibit the same self-association ability as their corresponding L-enantiomers; and the hemolytic activity and antimicrobial activity of D-peptides against human red blood cells and microbial cells, respectively, were quantitatively equivalent to those of the L-enantiomers. Thus, there is no chiral selectivity by the membrane or other stereoselective interactions in the cytoplasm with respect to the hemolytic and antimicrobial activities.

Because hemolytic activity is time dependent and there is no universal protocol for determining hemolytic activity, it is difficult to compare data from different sources. Hence, time course is important in the analysis of erythrocyte lysis. We have established a stringent criterion for nontoxicity: no hemolysis after 8 hours at a peptide concentration of 500 μg/ml. We believe that this timing and peptide concentration give a much more accurate evaluation of hemolytic activity (and toxicity to higher eukaryotic cells).

Peptides NA_{D} and NK_{L} were effective against a diverse group of Pseudomonas aeruginosa clinical isolates. Peptide D-NA_{L} exhibited the highest antimicrobial activity against Pseudomonas aeruginosa strains; in contrast, D-NK_{D} has the best overall therapeutic index due to its lack of hemolytic activity. Pseudomonas aeruginosa is a family of notorious Gram-negative bacterial strains which are resistant to many current antibiotics, thus, it is one of the most severe threats to human health (58-60). Only a few antibiotics are effective against Pseudomonas, including fluoroquinolones, gentamicin and imipenem, and even these antibiotics are not effective against all strains. In the studies disclosed herein, MIC values for Pseudomonas aeruginosa and other Gram-negative and Gram-positive bacteria were determined in two laboratories; in addition to different media used, the inoculum numbers of cells were also different (see details below), which may explain some variations of MIC values of Pseudomonas aeruginosa strains.

There is generally no significant difference in peptide antimicrobial activities against Pseudomonas aeruginosa strains, other Gram-negative and Gram-positive bacteria and a fungus between L- and D-enantiomeric peptides, or among peptides with different amino acid substitutions, i.e., V₆₈₁, NA_{D} and NK_{A}. There is a dramatic difference in peptide hydrophobicity at position 13 between Val and Lys. The Lys disrupts the continuous non-polar surface due to the positive charge and causes the peptide to locate in the interface region of the microbial membrane. This supports the view that the “carpet” mechanism is essential for strong antimicrobial activity, i.e., for both L- and D-peptide enantiomers, the peptides kill bacteria by a detergent-like mechanism, without penetrating deeply into the hydrophobic core of membrane.

D-peptides were resistant to enzymatic digestion; this may explain the slightly higher antimicrobial activity of D-peptides as compared to that of the L-enantiomer counterparts against Pseudomonas aeruginosa and Gram-positive bacteria. The relatively high susceptibility of L-peptides to trypsin is due to the presence of multiple lysine residues.

In summary, the earlier work showed that L- and D-enantiomeric peptide pairs behave similarly with respect to self-association in solution, in hemolytic activity against human red blood cells, and antimicrobial activity against Pseudomonas aeruginosa strains, and other Gram-negative and Gram-positive bacteria and a fungus. No chiral selectivity was found with respect to the antimicrobial and hemolytic activities of the peptides, supporting the “membrane discrimination” model as the mechanism of action for both L- and D-enantiomeric peptides.

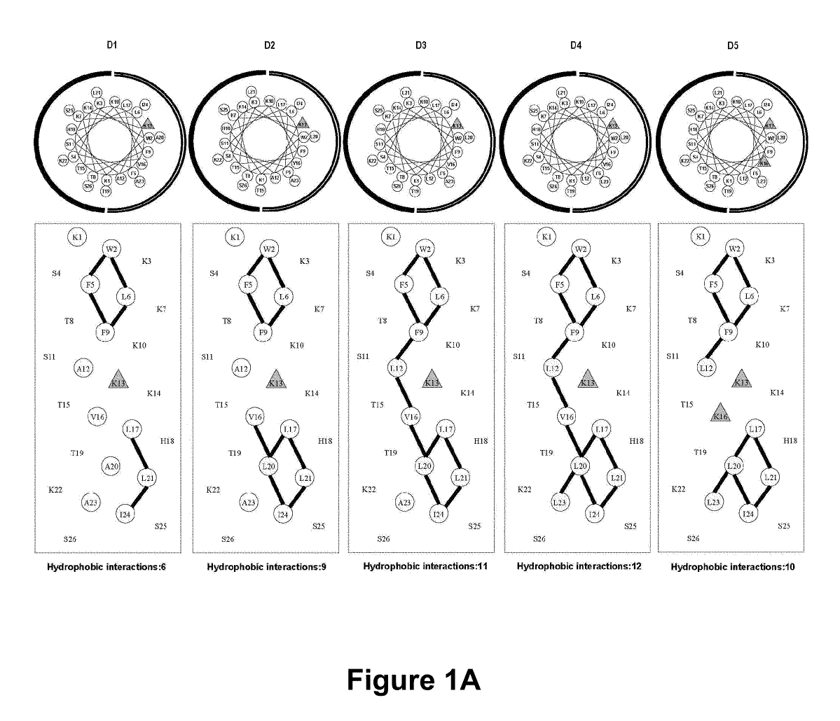

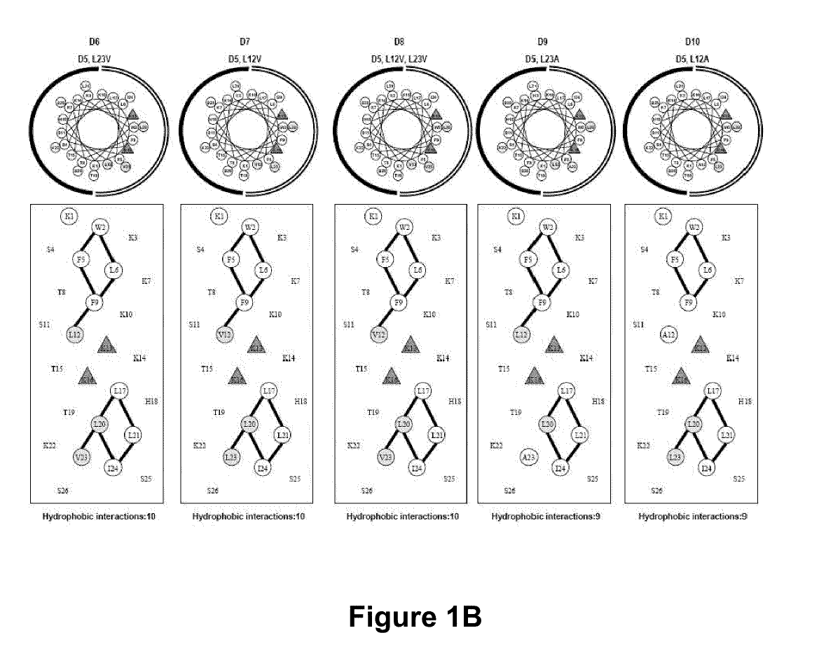

Similarly, peptides with all D-amino acid residues were more active against M. tuberculosis than peptides with all L-amino acid residues, at least in part because the D-peptides were more resistant to proteolytic enzymes in the capsule of M. tuberculosis. Therefore, peptides consisting of all D-amino acid residues were designed and synthesized. Peptide D-V13K (D1) (SEQ ID NO:24) is a 26-residue amphipathic peptide consisting of all D-amino acid residues. It adopts an α-helical conformation in a hydrophobic environment and contains a hydrophilic and positively-charged lysine residue in the center of the non-polar face (position 13) (Table 1, FIG. 1) (53, 118, 119). Herein we describe the results for systematically substituting one, two or three alanine residues with the more hydrophobic leucine residues to generate peptides D2, D3 and D4 (Table 4, FIG. 1, SEQ ID NOs:53-56). To increase the antimicrobial activity and decrease the high tendency for self-association (119), peptide D5 (SEQ ID NO:56) was designed with substitution of lysine for valine at position 16 of D4 (SEQ ID NO:55). This modification decreased hydrophobicity, amphipathicity, helicity, self-association and hemolytic activity of peptide D5 as compared to peptide D4, and antibacterial and antifungal activity were greater for D5 than D4.

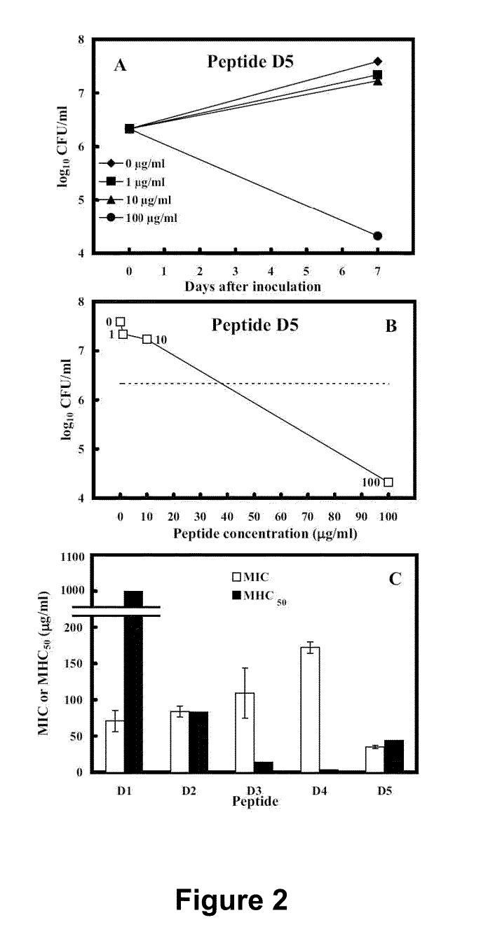

Anti-tuberculosis activities of the modified peptides described herein were determined. The time-course of antimycobacterial activity of peptide D5 (SEQ ID NO:56) was shown in FIG. 2A. 1, 10 and 100 μg/ml or 0.317, 3.17 and 31.7 μM, the 10-fold serial concentrations were used. After 7 days incubation with peptide D5, the colony-forming units/ml of each samples were calculated and compared with day 0. The sample treated with 100 μg/ml (31.7 μM) had dramatic a reduction in viability (CFU/ml) by about 100-fold, from 10^{6.33} to 10^{4.32}. The data were converted to a concentration-response format, and fit to a line (FIG. 2, Panel B). The point at which the line crossed the concentration of the initial inoculum (dashed line) was reported as the MIC. The MIC value of peptide D5 is 35.2±2.1 μg/ml or 11.2±0.7 μM, the most active in this series (FIG. 2, Panel C, Tables 4 and 5). The less active peptide is D4, with a MIC value of 55.1±2.9 μg/ml or 171.9±9.0 μM (Tables 4 and 5). Peptide D5 exhibits increased antimycobacterial activity by about 4.9-fold as compared to D4, (valine to lysine substitution at position 16 of D4). Our lead compound, peptide D1 had 2.4-fold improvement in anti-tuberculosis activity compared to that of D4 (Tables 4 and 5).

The hemolytic activities of the peptides for human erythrocytes were determined as a measure of toxicity toward higher eukaryotic cells. The MHC₅₀ values, the maximal peptide concentration that produces 50% hemolysis of human red blood cells after 18 hours in the standard microtiter dilution method, are shown in Tables 4 and 5 and FIG. 2, Panel C. From the strongest hemolytic peptide D4 (SEQ ID NO:55) to the weakest hemolytic peptide D1 (SEQ ID NO:24), there is a 286-fold difference in MHC₅₀ value. The most active peptide in antimycobacterial activity, D5 showed 13-fold improvement in hemolytic activity compared to D4; the only difference in sequence is at position 16: valine in D4 and lysine in D5 (SEQ ID NO:56), respectively.

The therapeutic indices are shown in Table 4 and Table 5. Large values indicate greater antimicrobial specificity than toxicity as measured by hemolytic assays. The best peptide is D1 (SEQ ID NO:24) with a therapeutic index value of 14.1; while the worst peptide is D4, SEQ ID NO:55, the most hydrophobic analog, with a therapeutic index value of 0.02. There is a 695-fold difference between them. However, the peptide with the strongest antimycobacterial activity is D5, which has a lysine at position 16, SEQ ID NO:56). The D5 peptide has a therapeutic index value of 1.3, a 61-fold improvement over D4 (valine at position 16, SEQ ID NO:55).

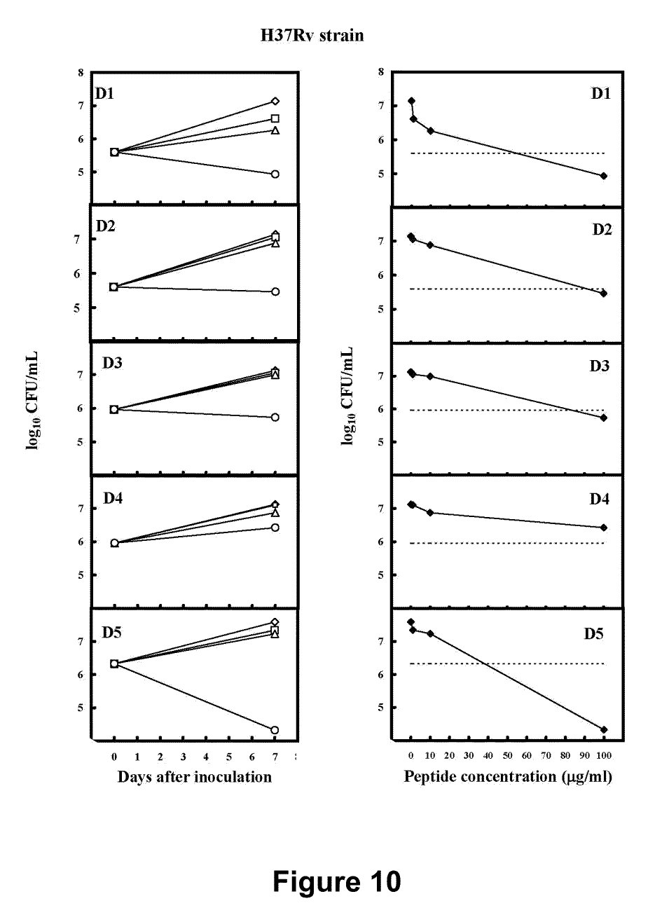

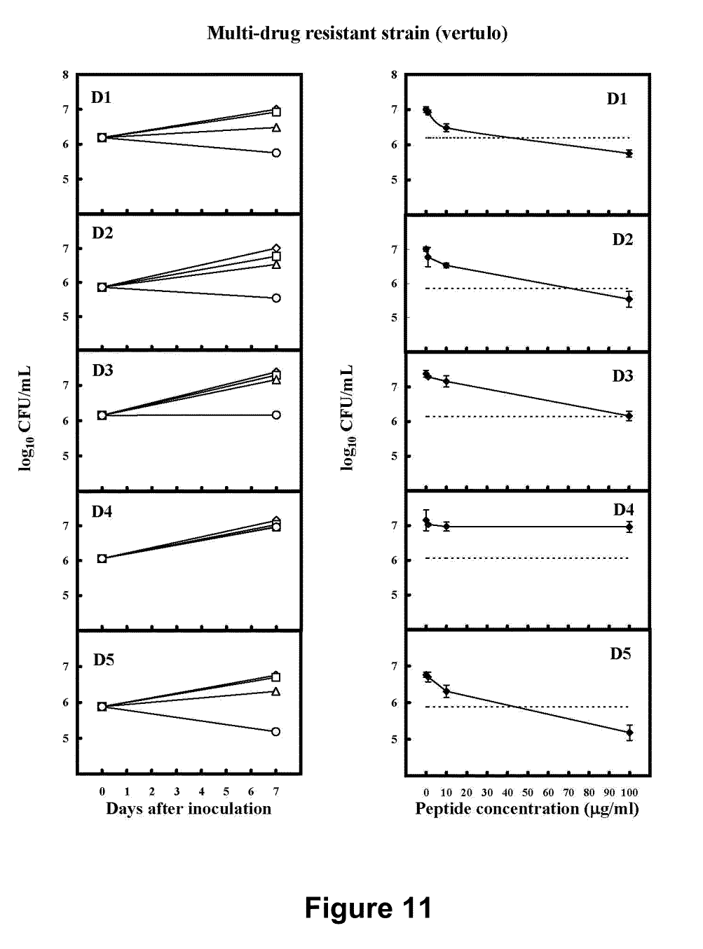

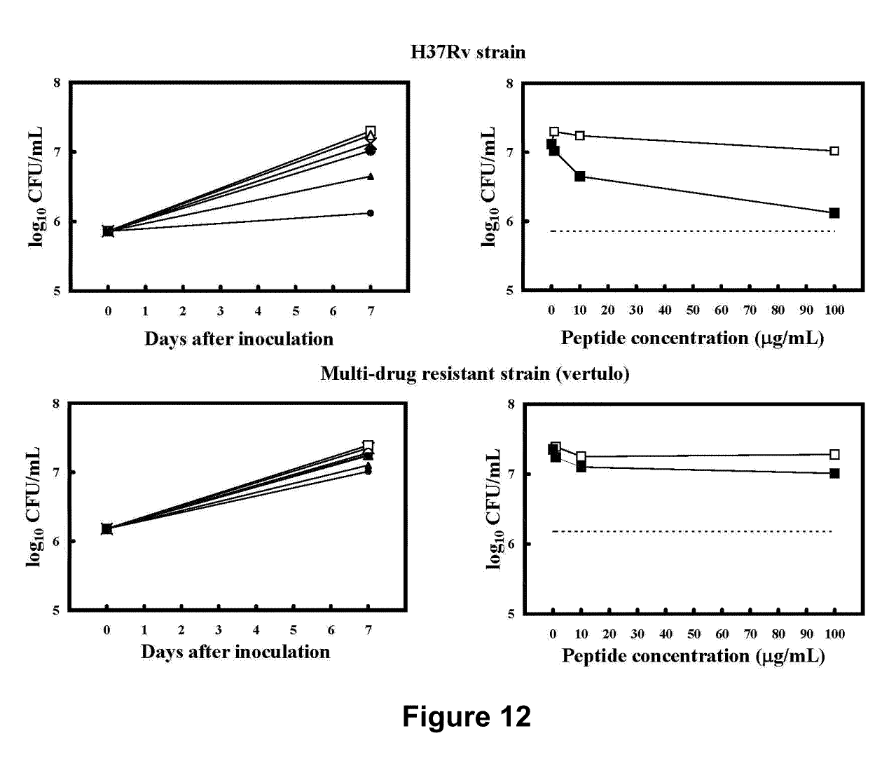

Additional experiments were carried out with M. tuberculosis and peptides D1-D5, using the H37Rv strain and the multiple drug resistance “vertulo” strain. See FIG. 10-12 and Table 5. Peptide D5 was confirmed to be the most active antimicrobial peptide in the present series against both a standard strain and the multiple drug resistance strain tested. However, peptide D1 is better with respect to therapeutic index. It is noted that the present antimicrobial peptides have stronger activity against M. tuberculosis than the human antimicrobial peptide LL-37, as disclosed by Martineau et al. (2007) J. Clin. Invest. 117:1988-1994.

Additional broad spectrum antimicrobial peptides are those sequences asset forth in SEQ ID NO:57-61 (D6, D7, 08, D9 and D10 respectively). Peptides D6-D8 have 10 hydrophobic interactions each, and D9-D10 have nine hydrophobic interactions each.

TABLE 3
Peptides used in this study

| | | | | |
|---|---|---|---|---|
| Peptide | SEQ | | | Hydrophobicity |
| Name | ID NO | Substitution^{a} | Sequence^{b} | t_{R}^{c} (min) |

| | | | | |
|---|---|---|---|---|
| D1 | 24 | D-(V13K) | Ac-KWKSFLKTFKSAKKTVLHTALKAISS-amide | 76.8 |
| D2 | 53 | D-(V13K, A20L) | Ac-KWKSFLKTFKSAKKTVLHTLLKAISS-amide | 86.7 |
| D3 | 54 | D-(V13K, A12L, A20L) | Ac-KWKSFLKTFKSLKKTVLHTLLKAISS-amide | 94.8 |
| D4 | 55 | D-(V13K, A12L, A20L, A23L) | Ac-KWKSFLKTFKSLKKTVLHTLLKLISS-amide | 101.6 |
| D5 | 56 | D-(V13K, A12L, A20L, A23L, | Ac-KWKSFLKTFKSLKKTKLHTLLKLISS-amide | 80.4 |
| | | V16K) | | |
| D6 | 57 | D-(V13K, V16K, A12L, A20L, | Ac-KWKSFLKTFKSLKKTKLHTLLKVISS-amide | |
| | | A23,V) | | |
| D7 | 58 | D-(V13K, V16K, A12V, A20L, | Ac-KWKSFLKTFKSVKKTKLHTLLKLISS-amide | |
| | | A23L) | | |
| D8 | 59 | D-(V13K, V16K, A12V, A20L, | Ac-KWKSFLKTFKSVKKTKLHTLLkVISS-amide | |
| | | A23V) | | |
| D9 | 60 | D-(V13K, V16K, A12V, A20L) | Ac-KWKSFLKTFKSLKKTKLHTLLKAISS-amide | |
| D10 | 61 | D-(V13K, V16K, A20L, A23L | Ac-KWKSFLKTFKSAKKTKLHTLLKLISS-amide | |

^{a}The D- denotes that all amino acid residues in each peptide are in the D conformation.
^{b}Peptide sequences are shown using the one-letter code for amino acid residues; Ac- denotes Nα-acetyl and -amide denotes C-terminal amide. The important substitutions on the nonpolar face are bolded.
^{c}t_{R} denotes retention time in RP-HPLC at pH 2 and room temperature, and is a measure of overall peptide hydrophobicity.

TABLE 4
Biological activity of D-(V13K) analogs against M. tuberculosis

| | | | | Anti-tuberculosis | | | | |
|---|---|---|---|---|---|---|---|---|
| | Hemolytic activity | | | activity | | | Therapeutic | |
| | MHC₅₀^{a} | | | MIC^{c} | | | index | |
| Peptide Name | μM | μg/ml | Fold^{b} | μM | μg/ml | Fold^{d} | MHC₅₀/MIC^{e} | Fold^{f} |
| D1 | 334 | 1000 | 286 | 23.6 ± 5.0 | 70.7 ± 14.8 | 2.3 | 14.1 | 695 |
| D2 | 27 | 83 | 24 | 27.6 ± 2.5 | 83.7 ± 7.7 | 2.0 | 1.0 | 49 |
| D3 | 5 | 14 | 4 | 35.5 ± 11.3 | 109.2 ± 34.8 | 1.6 | 0.1 | 6 |
| D4 | 1 | 3.5 | 1 | 55.1 ± 2.9 | 171.9 ± 9.0 | 1.0 | 0.02 | 1 |
| D5 | 14 | 44 | 13 | 11.2 ± 0.7 | 35.2 ± 2.1 | 4.9 | 1.3 | 61 |

^{a}MHC50 is the maximal peptide concentration that produces 50% hemolysis of human red blood cells after 18 h in the standard microtiter dilution method.
^{b}The fold improvement in MHC₅₀ compared to that of D4.
^{c}MIC (±standard deviation) is minimal inhibitory concentration that inhibited 99.9% growth of M. tuberculosis in killing assay. The MIC value of rifampin is 0.033 ± 0.005 μM (0.027 ± 0.004 μg/ml); the MIC value of isoniazid is 0.343 ± 0.07 μM (0.045 ± 0.01 μg/ml)
^{d}The fold improvement in anti-tuberculosis activity compared to that of D4.
^{e}Therapeutic index is the ratio of the MHC50 value over the geometric mean MIC value. Large values indicate greater antimicrobial specificity.
^{f}The fold improvement in therapeutic index compared to that of D4.

TABLE 5
Biological activity of D-(V13K) analogs against M. tuberculosis (further experiments)

| | | | | | | | | | Anti-tuberculosis | | | | |
|---|---|---|---|---|---|---|---|---|---|---|---|---|---|
| | | | | Anti-tuberculosis | | | | | activity to multi- | | | | |
| | | | | activity to | | | | | drug resistant | | | | |
| | Hemolytic activity | | | H37Rv strain | | | Therapeutic index | | strain (vertulo) | | | Therapeutic index | |
| Peptide | HC₅₀^{a} | | | MIC^{c} | | | to H37Rv strain | | MIC^{c} | | | to vertulo strain | |
| Name | μM | μg/ml | Fold^{b} | μM | μg/ml | Fold^{d} | HC₅₀/MIC^{e} | Fold^{f} | μM | μg/ml | Fold^{d} | HC₅₀/MIC^{e} | Fold^{f} |
| D1 | 140.9 | 421.5 | 120 | 23.6 | 70.7 | 2.3 | 6.0 | 293 | 19.1 | 57 | >8.4 | 7.4 | >1056 |
| D2 | 27.4 | 83 | 24 | 27.6 | 83.7 | 2.0 | 1.0 | 49 | 23.7 | 72 | >6.8 | 1.2 | >164 |
| D3 | 4.6 | 14 | 4 | 35.5 | 109.2 | 1.6 | 0.1 | 6 | 32.5 | 100 | >4.9 | 0.14 | >20 |
| D4 | 1.1 | 3.5 | 1 | 55.1 | 171.9 | 1.0 | 0.02 | 1 | >160.4 | >500 | 1 | <0.007 | 1 |
| D5 | 14.9 | 47 | 13 | 11.2 | 35.2 | 4.9 | 1.3 | 66 | 15.6 | 49 | >10.3 | 1.0 | >137 |

^{a}HC50 is the peptide concentration that produces 50% hemolysis of human red blood cells after 18 h in the standard microtiter dilution method.
^{b}The fold improvement in HC50 compared to that of D4.
^{c}MIC is minimal inhibitory concentration that inhibited 99.9% growth of M. tuberculosis in killing assay. The MIC value of rifampin is 0.033 ± 0.005 μM (0.027 ± 0.004 μg/ml); the MIC value of isoniazid is 0.343 ± 0.07 μM (0.045 ± 0.01 μg/ml)
^{d}The fold improvement in anti-tuberculosis activity compared to that of D4.
^{e}Therapeutic index is the ratio of the HC50 value over the geometric mean MIC value. Large values indicate greater antimicrobial specificity.
^{f}The fold improvement in therapeutic index compared to that of D4.

Hydrophobicity is a very important parameter with respect to antimicrobial activity (119, 95-97). In a previous study (119), we showed that an increase in hydrophobicity increases hemolytic activity, but there was an optimum hydrophobicity range over which high antimicrobial activity against Pseudomonas aeruginosa could be obtained. Altering hydrophobicity out of this window dramatically decreased antimicrobial activity. The decreased antimicrobial activity at high peptide hydrophobicity may be due to the strong tendency for self-association that prevents the peptide from crossing through the cell envelope in prokaryotic cells.

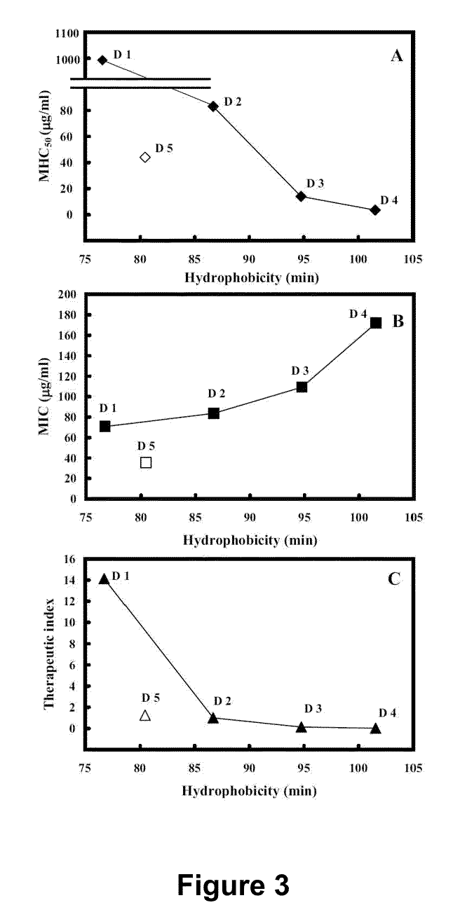

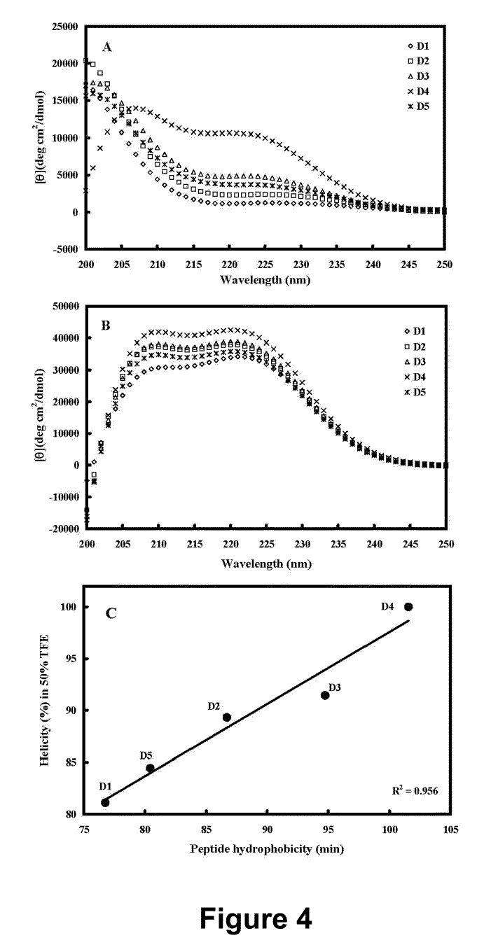

Reversed phase-HPLC(RP-HPLC) retention behavior is a particularly good method to evaluate peptide hydrophobicity; the retention times are highly sensitive to the conformational status of peptides upon interaction with the hydrophobic environment of the column matrix (1,8). The nonpolar face of an amphipathic α-helical peptide represents a preferred binding domain for interaction with the hydrophobic matrix of a reversed-phase column. The response of MHC₅₀ value (hemolytic activity), MIC value (antimycobacterial activity) and therapeutic index (antimicrobial specificity) to the increase of hydrophobicity (expressed as RP-HPLC retention time, Table 6) was plotted in FIG. 3, Panels A, B, and C, respectively. For peptide D1 to D4, increasing hydrophobicity dramatically increased hemolytic activity (FIG. 3, Panel A) up to 286-fold (Table 4), whereas decreased antimycobacterial activity (FIG. 3B) up to 2.3-fold (Table 4). As a result, it decreased antimicrobial specificity (therapeutic index) up to 695-fold (Table 4). Triple-Leu-substituted peptide D4 showed the highest hydrophobicity among the peptide analogs (tR=101.6 min; Table 6). By replacing one extra valine with lysine at position 16, the hydrophobicity decreased from 101.6 min for D4 to 80.4 min for D5, i.e., the effect of a triple Ala→Leu substitution on hydrophobicity (D1→D4; hydrophobicity values of 76.8 min and 101.6 min, respectively, for an increase of 24.8 min) was essentially overridden by only a single Val→Lys substitution (D5→D4; a decrease in hydrophobicity of 21.2 min). It should be noted, however, that although the overall hydrophobicity of D5 is dramatically decreased compared to D4 due to the presence of the extra Lys residue, the Leu residues are still increasing the hydrophobicity of the two individual hydrophobic segments. The similar result was observed for P. aeruginosa, due to the disadvantageous self-association associated with higher hydrophobicity. By converting one valine to lysine at position 16 of D4 (SEQ ID NO:55) to generate D5 (SEQ ID NO:56), the hydrophobicity decreased from 101.6 min to 80.4 min (Table 6), antimycobacterial activity increased 4.9-fold with hemolytic activity decreased 13-fold and therapeutic index increased 61-fold (Tables 4 and 5). This substitution decreased hydrophobicity and self-association but retained the high antimycobacterial activity and decreased hemolytic activity as compared to peptide D4.

These observations are consistent with the membrane discrimination mechanism (117-119). It demonstrated that the pore-formation mechanism (“barrel-stave” mechanism (45,98) was applied to antimicrobial peptides interacting with zwitterionic eukaryotic membranes, while the detergent-like mechanism (“carpet” mechanism; 46) was applied to antimicrobial peptides interacting with negatively charged prokaryotic membranes. Peptides with higher hydrophobicities penetrate deeper into the hydrophobic core of the red blood cell membrane (67), causing stronger hemolysis by forming pores or channels. However, there is no such insertion involved in the interaction between antimicrobial peptides and bacterial membrane; antimicrobial activity would not increase with the increasing of hydrophobicity. But the unwanted high level of peptide self-association resulting from higher hydrophobicity prevented the highly folded and dimerized/oligomerized peptides from passing through the cell envelope, thus decreased their antimicrobial activity. The valine to lysine substitution in the D5 peptide decreased the hydrophobicity and disrupted the consistency of hydrophobic surface (FIG. 1), thus decreasing hemolytic activity and self-association, and increasing antimicrobial activity.

Antimicrobial peptides consisting of all L-amino acids can be susceptible to proteolytic degradation by enzymes produced by the organism one is trying to kill. All-D-peptides are resistant to proteolytic enzyme degradation which enhances their potential as clinical therapeutics, but all-D-peptides can only be used where the antimicrobial mechanism of action does not involve a stereoselective interaction with a chiral enzyme or lipid or protein receptor. For antimicrobial peptide V13K, its all-L-form (L-V13K; SEQ ID NO:6) and all-D-form (D1, D-V13K; SEQ ID NO:24) were equally active, suggesting that the sole target for these peptides was the membrane (92). The parent peptide used in this study was D-V13K (D1; SEQ ID NO:24), a 26-residue amphipathic peptide consisting of all D-amino acid residues, which adopts an α-helical conformation in a hydrophobic environment and contains a hydrophilic, positively-charged lysine residue in the center of the non-polar face (position 13) (FIG. 1) (52, 92, 93). In the present study, we used peptide D-V13K (SEQ ID NO:24) as a framework to alter peptide hydrophobicity systematically on the nonpolar face of the helix by replacing one (peptide D2; SEQ ID NO:53), two (D3; SEQ ID NO:54) or three (D4; SEQ ID NO:55) alanine residues with more hydrophobic leucine residues to increase hydrophobicity. The peptide sequences are shown in Table 1, with helical wheel and helical net representations shown in FIG. 1. The number of i→i+3 and i→i+4 hydrophobic interactions on the nonpolar face (a peptide sequence in an α-helical conformation allows a side-chain in position i to interact with a side-chain in position i+3 or i+4 along the sequence) increases with the addition of leucine residues (6 for D1, 9 for D2, 11 for D3 and 12 for D4) (FIG. 1).

It was previously shown that placement of a positively charged residue in the center of the non-polar face of amphipathic α-helical and cyclic β-sheet (20) antimicrobial peptides is a determinant of specificity between eukaryotic and prokaryotic cells; increasing hydrophobicity over an optimum value decreased antibacterial activity because of strong peptide self-association, which we proposed prevents the peptide from passing through the cell wall to reach the membrane in prokaryotic cells, while increasing hydrophobicity increases hemolytic activity; and increased peptide self-association had no effect on peptide access to eukaryotic membranes. Based on these observations, we hypothesize that the optimum therapeutic index could be achieved by increasing hydrophobicity to increase antimicrobial activity and maintaining poor hemolytic activity by the addition of an extra positive charge in the center of the nonpolar face. Thus, we designed peptide D5 (D-(V13K, A12L, A20L, A23L, V16K; SEQ ID NO:56)) by replacing the hydrophobic valine residue at position 16 with a positively-charged lysine residue to give two lysine residues in the center of the nonpolar face (positions 13 and 16) (FIG. 1). This additional positive charge would further disrupt the consistency of the hydrophobic surface, and prevent the high-level of self-association observed with peptide D4. This V16K substitution was designed to allow the increased hydrophobicity (A12L, A20L, A23L) to enhance antimicrobial activity without increasing hemolytic activity. In this case, the number of and i→i+3 and i→i+4 potential hydrophobic interactions decreased from 12 for D4 to 10 for D5, with the continuous hydrophobic face of D4 now disrupted into two separate hydrophobic segments in D5 (FIG. 1).

The sequence of D1, even with a lysine residue in the center of the nonpolar face is still very amphipathic with a value of 4.92 (Table 8). There is an increase in amphipathicity as hydrophobicity is systematically increased. The amphipathicity of our analogs ranged from 4.92 to 6.34 (Table 8). By replacing valine with lysine (V16K), the amphipathicity only decreased from 6.34 for D4 to 5.78 for D5.

As previously shown (92), the L- and D-enantiomers of peptide V13K had equal activities, and the all-D peptides, were resistant to proteolytic enzyme degradation. The all-D peptides proved to be more active against fungi than their L-enantiomers. Without wishing to be bound by theory, it is believed that this is due to the resistance to proteolytic enzymes in the fungal cell envelopes.

The secondary structure of the peptides was studied. FIG. 5 shows the CD spectra of the peptide analogs in different environments, i.e., under benign conditions (non-denaturing) (FIG. 5, Panel A) and in buffer with 50% TFE to mimic the hydrophobic environment of the membrane (FIG. 5, Panel B). It should be noted that all-D helical peptides will exhibit a positive spectrum while all-L helical peptides will exhibit a negative spectrum (92). All peptides except D4 exhibited negligible secondary structure in benign buffer (FIG. 5, Panel A and Table 8). D4, the triple-Leu-substituted peptide, exhibited an α-helix spectrum under benign conditions (25% α-helix, Table 8) compared to the spectra of the other analogs. Regardless of the different secondary structures of the peptides in benign buffer, a highly helical structure was induced by the nonpolar environment of 50% TFE, a mimic of hydrophobicity and the α-helix-inducing ability of the membrane (FIG. 5B and Table 6). All the peptide analogs showed a typical α-helix spectrum with double maxima at 208 nm and 222 nm. The helicities of the peptides in benign buffer and in 50% TFE relative to that of peptide D4 in 50% TFE were determined (Table 6). From FIG. 5, Panel C, it is clear that increasing peptide hydrophobicity linearly correlates with increasing α-helical structure of the peptides in hydrophobic (50% TFE) environments (R²=0.956).

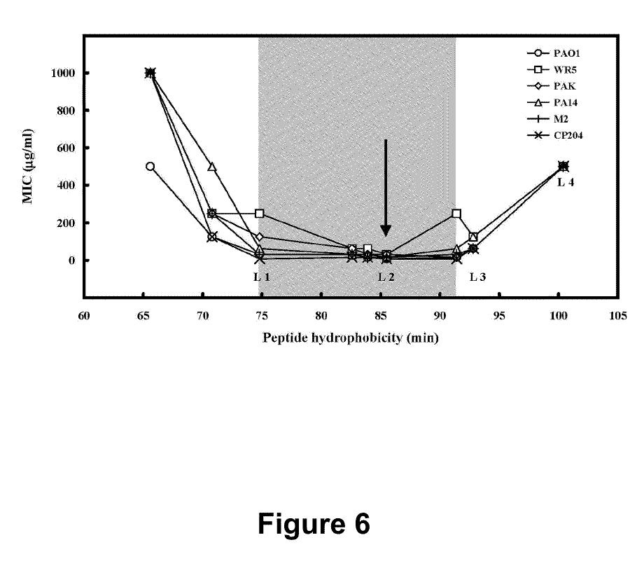

Peptide self-association (i.e., the ability to oligomerize/dimerize) in aqueous solution is a very important parameter for antimicrobial activity (53, 92-93). We postulated that monomeric random-coil antimicrobial peptides are best suited to pass through the capsule and cell wall of microorganisms prior to penetration into the cytoplasmic membrane, induction of α-helical structure and disruption of membrane structure to kill target cells (93). Thus, if the self-association ability of a peptide in aqueous media is too strong (e.g., forming stable folded dimers through interaction of their non-polar faces) this could decrease the ability of the peptide to dissociate to monomer where the dimer cannot effectively pass through the capsule and cell wall to reach the membrane. The ability of the peptides in the present study to self-associate was determined by the technique of RP-HPLC temperature profiling at pH 2 (30,29,42). The reason pH 2 is used to determine self-association of cationic AMPs is that highly positively charged peptides are frequently not eluted from reversed-phase columns at pH 7 due to non-specific binding to negatively charged silanols on the column matrix. This is not a problem at pH 2 since the silanols are protonated (i.e., neutral) and non-specific electrostatic interactions are eliminated. At pH 2, the interactions between the peptide and the reversed-phase matrix involve ideal retention behavior, i.e., only hydrophobic interactions between the preferred binding domain (nonpolar face) of the amphipathic molecule and the hydrophobic surface of the column matrix are present (39). FIG. 6A shows the retention behavior of the peptides after normalization to their retention times at 5° C. Control peptide C shows a linear decrease in retention time with increasing temperature and is representative of peptides which have no ability to self-associate during RP-HPLC. Control peptide C is a monomeric random coil peptide in both aqueous and hydrophobic media; thus, its linear decrease in peptide retention behavior with increasing temperature within the range of 5° C. to 80° C. represents only the general effects of temperature due to greater solute diffusivity and enhanced mass transfer between the stationary and mobile phase at higher temperatures (55). To allow for these general temperature effects, the data for the control peptide was subtracted from each temperature profile as shown in FIG. 6B. Thus, the peptide self-association parameter, P_{A}, represents the maximum change in peptide retention time relative to the random coil peptide C. Note that the higher the P_{A} value, the greater the self-association.

By replacing a single valine with lysine in the center of the nonpolar face (V13K, D1 in the present study), there was a dramatic decrease in self-association. However, by systematically increasing the hydrophobicity of the nonpolar face (from peptide D1 to D4), the self-association ability also increased (FIG. 6, Panel C shows a linear increase in self-association ability with increasing hydrophobicity of the non-polar face (R²=0.966). By replacing a second valine with lysine in the center of the nonpolar face (position 16) of D4 generating D5, there was a dramatic decrease in self-association ability (FIG. 6, Panel B), i.e., the substantial positive effect of a triple Ala→Leu substitution (D4) on self-association was overridden by a single V16K substitution (D5; SEQ ID NO:56). Thus, peptide D5 maintains the three Leu residues and an increase in hydrophobicity in the two hydrophobic patches (FIG. 1) while maintaining low self-association compared to peptide D4 (Table 6, FIG. 6, Panel C).

TABLE 6
Biophysical data for D-(V13K) analogs

| Peptide | | Hydrophobicity | Benign | | 50% TFE | | |
|---|---|---|---|---|---|---|---|
| Name | Amphipathicity^{a} | t_{R}^{b} (min) | [θ]₂₂₂^{c} | % Helix^{d} | [θ]₂₂₂^{c} | % Helix^{d} | P_{A}^{e} |
| D1 | 4.92 | 76.8 | 1,150 | 3 | 34,100 | 81 | 2.78 |
| D2 | 5.71 | 86.7 | 2,300 | 5 | 37,550 | 89 | 4.62 |
| D3 | 5.86 | 94.8 | 4,850 | 12 | 38,450 | 91 | 7.67 |
| D4 | 6.34 | 101.6 | 10,550 | 25 | 42,050 | 100 | 9.63 |
| D5 | 5.78 | 80.4 | 3,700 | 9 | 35,500 | 84 | 4.35 |

^{a}Amphipathicity of peptide analogs was determined by calculation of hydrophobic moment (32) using the software package Jemboss version 1.2.1 (33), modified to include a hydrophobicity scale determined in our laboratory at pH 7 (54).
^{b}t_{R} denotes retention time in RP-HPLC at pH 2 and room temperature, and is a measure of overall peptide hydrophobicity.
^{c}The mean residue molar ellipticities [θ]₂₂₂ (deg cm²/dmol) at wavelength 222 nm were measured at 5 □ in benign conditions (100 mM KCl, 50 mM NaH₂PO₄/Na₂HPO₄, pH 7.0) or in benign buffer containing 50% trifluoroethanol (TFE) by circular dichroism spectroscopy.
^{d}The helical content (as a percentage) of a peptide relative to the molar ellipticity value of peptide D4 in the presence of 50% TFE.
^{e}P_{A} denotes dimerization parameter of each peptide during RP-HPLC temperature profiling, which is the maximal retention time difference of (t_{R}^{t} − t_{R}⁵ for peptide analogs) − (t_{R}^{t} − t_{R}⁵ for control peptide C) within the temperature range; t_{R}^{t} − t_{R}⁵ is the retention time difference of a peptide at a specific temperature (t_{R}^{t}) compared with that at 5° C. (t_{R}⁵). The sequence of control peptide C is Ac-E

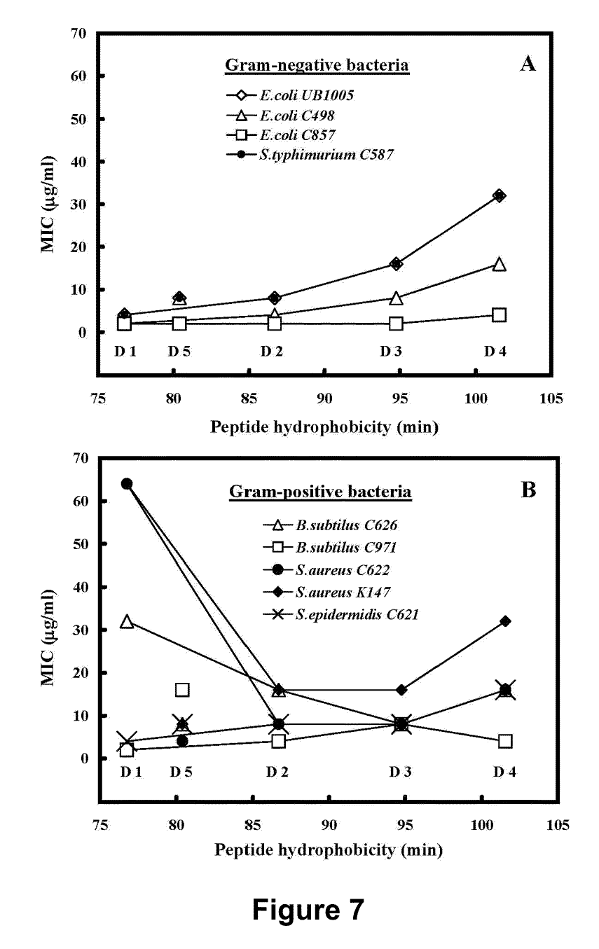

From our previous studies, the all-L forms of our peptide analogs (L1, L2, L3 and L4, with the same sequences as D1, D2, D3 and D4, respectively) showed an optimum hydrophobicity on the non-polar face for best antimicrobial activity (indicated by the arrow) against six clinical-isolate strains of Pseudomonas aeruginosa (93) (FIG. 7). Increasing hydrophobicity beyond the optimum value dramatically decreased antimicrobial activity (peptide L4, FIG. 7). Similarly, decreasing the hydrophobicity beyond peptide L1 dramatically decreased antimicrobial activity. Thus, there is a window of hydrophobicity (indicated by the shaded area in FIG. 7) for maintaining good antimicrobial activity. This window of hydrophobicity allows one to select the peptide hydrophobicity that provides the best therapeutic index (see hemolytic activities described below).

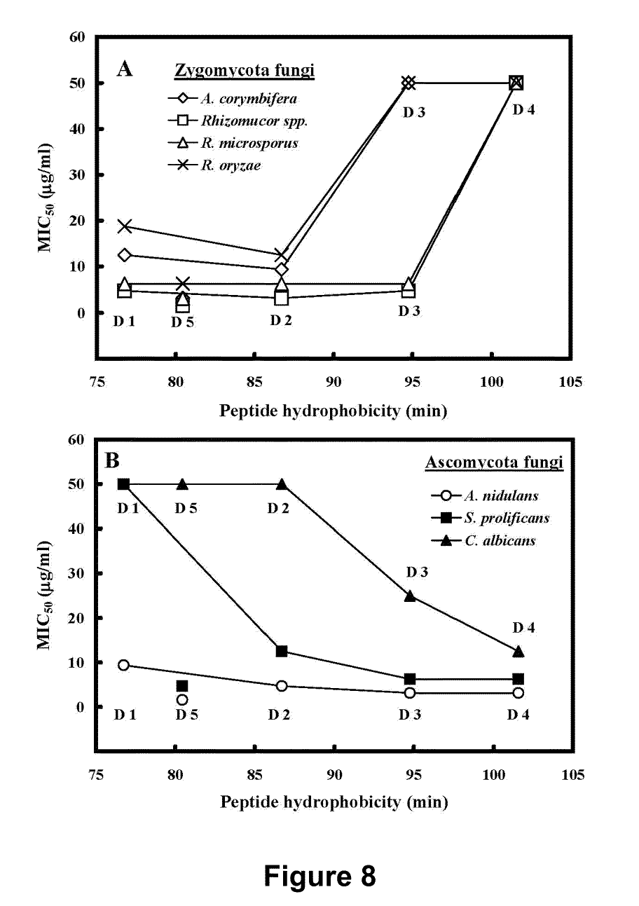

The antibacterial activities against six gram-negative bacteria/strains and six gram-positive bacteria/strains are compared in Table 9. Geometric mean of MIC was calculated to provide an overall view of antimicrobial activity of different analogs. It is clear that our peptides were effective in killing the microorganisms tested. The tested gram-negative bacteria showed a similar correlation between MIC values and peptide hydrophobicity (FIG. 7, Panel A) as seen previously for P. aeruginosa (FIG. 7): increasing the peptide hydrophobicity from 76.8 min for D1 to 101.6 min for D4 resulted in a reduction in antibacterial activity, albeit the magnitude of the effect differed for each bacterium/strain; for instance, little change was seen for E. coli C857 over the entire range of peptide hydrophobicity for D1 to D4. For the gram-positive bacteria (FIG. 8, Panel B), the results were more complex, with antibacterial activity for three of the bacteria first increasing with increasing peptide hydrophobicity and then decreasing with a further increase in hydrophobicity. However, one of the bacteria (B. subtilis C971) showed relatively little change over the hydrophobicity range. By replacing another valine with lysine in the center of the nonpolar face (position 16), D5 exhibited an increase in antibacterial activity 2-fold greater compared to D4 for gram-negative and gram-positive bacteria (Table 7). It should be noted that the effects of hydrophobicity for peptide L4 (FIG. 6) was an order of magnitude greater than the effects of increasing hydrophobicity on the gram-negative and gram-positive bacteria shown in FIG. 7.

### Antifungal Activity

MIC₅₀ values, the minimal inhibitory concentration of peptide that inhibits 50% of fungal growth, were evaluated for seven pathogenic fungal strains (Table 8): both filamentous fungi (A. nidulans, A. corymbifera, Rhizomucor spp., R. microsporus, R. oryzae, S. prolificans) and encapsulated yeast (C. albicans). A. corymbifera, Rhizomucor spp., R. microsporus and R. oryzae belong to the phylum Zygomycota and can cause zygomycosis; A. nidulans, S. prolificans and C. albicans belong to the phylum Ascomycota and cause aspergillosis, Ascomycota and candidiasis, respectively.

FIG. 8, Panel A shows the relationship between MIC50 values for Zygomycota fungi and peptide hydrophobicity. A systematic increase in hydrophobicity (from peptide D1 to D4) resulted in a 5.5-fold reduction in antifungal activity (FIG. 8, Panel A, Table 8). However, for the ascomycotes fungi tested, the same series of peptides generated different results: increasing peptide hydrophobicity generally led to a continuous increase in antifungal activity with peptide D4 having a 5-fold increase in antifungal activity over peptide D1 (FIG. 8, Panel B, Table 8).

TABLE 7
Biological activity of D-(V13K) analogs against different Gram-negative (A), and Gram-positive (B) bacteria^{a}
A

| | | | Antimicrobial activity against Gram-negative bacteria | | | | | | | | | |
|---|---|---|---|---|---|---|---|---|---|---|---|---|
| | Hemolytic | | MIC^{d} (μg/ml) | | | | | | | | | |
| | activity | | | | E. coli | E. coli | | S. ryphimurium | | | | |
| Peptide | HC₅₀^{b} | | P. aeruginosa | E. coli | C498 | C857 | S. ryphimurium | C587 | | | Therapeutic index | |
| Name | (μg/ml) | Fold^{c} | PAO1 | UB1005 | UB1005 | DH5a | 14208S | 14208S | GM^{e} | Fold^{f} | HC₅₀/MIC^{g} | Fold^{h} |
| D1 | 1000 | 286 | 8 | 4 | 2 | 2 | 32 | 4 | 5.0 | 4.5 | 198.4 | 1281 |
| D2 | 83 | 24 | 8 | 8 | 4 | 2 | 64 | 8 | 8.0 | 2.8 | 10.4 | 67 |
| D3 | 14 | 4 | 16 | 16 | 8 | 2 | 64 | 16 | 12.7 | 1.8 | 1.1 | 7 |
| D4 | 3.5 | 1 | 16 | 32 | 16 | 4 | >64 | 32 | 22.6 | 1.0 | 0.2 | 1 |
| D5 | 44 | 13 | 8 | 8 | 8 | 2 | >64 | 8 | 10.1 | 2.2 | 4.4 | 28 |

B

| | | | Antimicrobial activity against Gram-positive bacteria | | | | | | | | | |
|---|---|---|---|---|---|---|---|---|---|---|---|---|
| | Hemolytic | | MIC^{d} (μg/ml) | | | | | | | | | |
| | activity | | | S. aureus | | | B. subtilus | | | | | |
| Peptide | HC₅₀^{b} | | S. aureus | C622 | S. epidermidis | B. subtilus | C971 | E. jaecalis | | | Therapeutic index | |
| Name | (μg/ml) | Fold^{c} | K147 | ATCC3923 | C623 | C626 | ATCC6633 | C625 | GM^{e} | Fold^{f} | HC₅₀/MIC^{g} | Fold^{h} |
| D1 | 1000 | 286 | 64 | 64 | 4 | 32 | 2 | 32 | 18.0 | 0.9 | 55.7 | 255 |
| D2 | 83 | 24 | 16 | 8 | 8 | 16 | 4 | 16 | 10.1 | 1.6 | 8.2 | 38 |
| D3 | 14 | 4 | 16 | 8 | 8 | 8 | 8 | 32 | 11.3 | 1.4 | 1.2 | 6 |
| D4 | 3.5 | 1 | 32 | 16 | 16 | 16 | 4 | 32 | 16.0 | 1.0 | 0.2 | 1 |
| D5 | 44 | 13 | 8 | 4 | 8 | 8 | 16 | 16 | 9.0 | 1.8 | 4.9 | 22 |

^{a}Antibacterial activity is given as mean value of 4 sets of determinations. We also included different cultures of the same strains.
^{b}HC₅₀ is the maximal peptide concentration that produces 50% hemolysis of human red blood cells after 18 h in the standard microtiter dilution method.
^{c}The fold improvement in HC₅₀ compared to that of D4.
^{d}MIC is minimal inhibitory concentration that inhibited growth of different strams in Mueller-Hmton(MH) medium at 37° C. after 24 h. MIC is given based on four sets of determinations.
^{e}GM. geometric mean of the MIC values. When no detectable antimicrobial activity was observed at 64 μg/mL. a value of 128 μg/mL was used for calculation of the GM value.
^{f}The fold improvement in antimicrobial activity (geometric mean data) compared to that of D4.
^{g}Therapeutic index is the ratio of the HC₅₀ value (μg/mL) over the geometric mean MIC value (μg/mL). Large values indicate greater antimicrobial specificity.
^{h}The fold improvement in therapeutic index compared to that of D4.

With the extra valine to lysine substitution in the center of the nonpolar face (position 16) of D4 (SEQ ID NO:55) to generate D5 (SEQ ID NO:56), antifungal activity increased by 16-fold for Zygomycota fungi and maintained the same level for Ascomycota fungi (Table 8). Overall, D5 is the best analog in our series for most of the tested fungal strains.

The hemolytic activities of the peptides against human erythrocytes were determined as a measure of peptide toxicity toward higher eukaryotic cells. The effect of peptide concentration on erythrocyte hemolysis is shown in FIG. 9, Panel A. From these plots the peptide concentration that produced 50% hemolysis was determined (HC₅₀). D4 showed the strongest hemolytic activity, while D1 showed the weakest. Hemolytic activity for peptides D2, D3, D4 and D5 increased in a hyperbolic fashion with increasing peptide concentration and all plateaued at 100% lysis when the peptide concentration was high enough. By comparison, hemolytic activity for peptide D1 increased in a linear fashion with increasing peptide concentration (FIG. 9, Panel A).

Hemolytic activity represented as HC₅₀ is shown in Table 8 and FIG. 9, Panel B. Increasing peptide hydrophobicity by replacing one, two or three alanine residues with leucine residues, decreased the HC₅₀ values from 1000 μg/ml for D1 to 83 μg/ml, 14 μg/ml and 3.5 μg/ml for D2, D3 and D4 respectively (Table 6, FIG. 9, Panel B), i.e., a 286-fold increase in hemolysis compared to that of the parent peptide, D1. By replacement of a second valine with lysine at position 16, to produce D5, hemolytic activity was decreased by 13-fold relative to D4 (from 3.5 μg/ml for D4 to 44 μg/ml for D5).

The therapeutic indices of the peptides D1-D5 for the fungal strains tested are shown in Table 10. The geometric mean MIC₅₀ values for Zygomycota and Ascomycota fungi was used to give an overall view of therapeutic index in fungi. Compared to that of the parent peptide, D1 ( ) SEQ ID NO:24), triple-Leu-substituted peptide D4 (SEQ ID NO:55) showed a decrease in therapeutic index by more than 1569-fold and 62-fold for Zygomycota and Ascomycota fungi, respectively, relative to peptide D4. Replacing a second valine with lysine at position 16 (D5) increased the therapeutic index by more than 200-fold and 11-fold for Zygomycota and Ascomycota fungi, respectively. Zygomycotes and ascomycotes exhibited different responses in MIC₅₀ to an increase in peptide hydrophobicity (FIG. 8); however, with the factor of hemolytic activity, the therapeutic index of both zygomycotes and ascomycotes express similar responses to an increase in peptide hydrophobicity.

TABLE 8
Biological activity of D-(V13K) analogs against Zygomycota fungi (A) and Ascomycota fungi (B) strains^{a}
A

| | | | Antifungal activity against Zygomycota fungi (μg/ml) | | | | | | | | | | | |
|---|---|---|---|---|---|---|---|---|---|---|---|---|---|---|
| Peptide | Hemolytic activity | | A. corymbifera | | Rhizomucor spp. | | R. micosporus | | R. oryzae | | | | Therapeutic index | |
| Name | HC₅₀^{b} (μg/ml) | Fold^{c} | MIC₅₀ | MIC₉₀ | MIC₅₀ | MIC₉₀ | MIC₅₀ | MIC₉₀ | MIC₅₀ | MIC₉₀ | GM^{e} | Fold^{f} | HC₅₀/MIC₅₀^{g} | Fold^{h} |
| D1 | 1000.0 | 286 | 12.5 | 12.5 | 4.7 | 6.3 | 6.3 | 12.5 | 18.8 | 25.0 | 9.1 | 5.5 | 109.9 | 1569 |
| D2 | 83 | 24 | 9.4 | 12.5 | 3.1 | 3.1 | 6.3 | 6.3 | 12.5 | 25.0 | 6.9 | 7.2 | 12.0 | 171 |
| D3 | 14 | 4 | 50.0 | 50.0 | 4.7 | 50.0 | 6.3 | 6.3 | 50.0 | 50.0 | 16.5 | 3.0 | 0.9 | 12 |
| D4 | 3.5 | 1 | 50.0 | 50.0 | 50.0 | 50.0 | 50.0 | 50.0 | 50.0 | 50.0 | 50.0 | 1.0 | 0.07 | 1 |
| D5 | 44 | 13 | 3.1 | 3.1 | 1.6 | 1.6 | 3.1 | 3.1 | 6.3 | 6.3 | 3.1 | 16.0 | 14.1 | 201 |

B

| | | | Antifungal activity against Ascomycota fungi (μg/ml) | | | | | | | | | |
|---|---|---|---|---|---|---|---|---|---|---|---|---|
| | Hemolytic activity | | A. nidulans | | S. prolificans | | C. albicans | | | | Therapeutic index | |
| Peptide Name | HC₅₀^{b} (μg/ml) | Fold^{c} | MIC₅₀ | MIC₉₀ | MIC₅₀ | MIC₉₀ | MIC₅₀ | MIC₉₀ | GM^{e} | Fold^{f} | HC₅₀/MIC₅₀^{g} | Fold^{h} |
| D1 | 1000.0 | 286 | 9.4 | 50.0 | 50.0 | 50.0 | 50.0 | 50.0 | 28.6 | 0.2 | 34.9 | 62 |

^{a}Antifungal activity is given as mean value of 2 sets of determinations.
^{b}HC₅₀ is the maximal peptide concentration that produces 50% hemolysis of human red blood cells after 18 h in the standard microtiter dilution method.
^{c}The fold improvement in HC₅₀ compared to that of D4.
^{d}MIC₅₀ (or MIC₉₀) are the defined as the peptide concentration (μg/ml) that inhibits 50% (or 90%) of fungal growth.
^{e}GM. geometric mean of the MIC₅₀ values.
^{f}The fold improvement in antifungal activity (geometric mean data) compared to that of D4.
^{g}Therapeutic index is the ratio of the HC₅₀ value (μg/mL) over the geometric mean MIC₅₀ value (μg/mL). Large values indicate greater antimicrobial specificity.
^{h}The fold improvement in therapeutic index compared to that of D4.

The therapeutic indices for different bacterial strains are shown in Table 7. Peptide D4 (SEQ ID NO:55), with the highest hydrophobicity among all analogs, exhibits the lowest therapeutic index: about 0.2 for both gram-negative bacteria and gram-positive bacteria. For peptide D5 (SEQ ID NO:56), the therapeutic index increased by 28- and 22-fold relative to peptide D4 for gram-negative and gram-positive bacteria, respectively.

Certain antibacterial and antifungal agents stimulate cytokine production, which could have potentially serious side-effects in patients. Thus, the D1-D5 peptides were tested for increased production of tumor necrosis factor (TNF) and interleukin-6 (IL-6) (Table 9). There was only a very low stimulation of IL-6 production when very high concentrations of some of the peptides were used. However, for the positive control (LPS stimulation, a standard cytokine inducer), a 1000-fold lower concentration than the peptides would give 10- to 100-fold higher cytokine stimulation. Thus, the peptides are very ineffective at stimulating cytokine production and even if very high concentrations of some (not all) of the peptides are used, a patient would be expected to exhibit only a slight febrile reaction, as is seen with other medications such as Amphotericin B and interferon-gamma, among others.

TABLE 9
Cytokine assay of D-(V13K) analogs
A

| | Peptide | | | | |
|---|---|---|---|---|---|
| | Concentration | TNF (ng/ml) | | IL-6 (pg/ml) | |
| Peptide Name | (μg/ml) | Exp 1 | Exp 2 | Exp 1 | Exp 2 |
| RPMI media | | <0.015 | <0.015 | <3 | 18 |
| (background) | | | | | |
| D1 | 100 | <0.015 | <0.015 | <3 | 570 |
| | 1 | <0.015 | <0.015 | 4 | 8 |
| | 0.01 | <0.015 | <0.015 | <3 | <3 |
| D2 | 100 | <0.015 | <0.015 | <3 | <3 |
| | 1 | <0.015 | <0.015 | 121 | 5 |
| | 0.01 | <0.015 | <0.015 | <3 | 3 |
| D3 | 100 | <0.015 | 0.05 | <3 | 333 |
| | 1 | <0.015 | <0.015 | <3 | 18 |
| | 0.01 | <0.015 | 0.05 | <3 | <3 |
| D4 | 100 | 0.04 | <0.015 | <3 | 216 |
| | 1 | 0.025 | <0.015 | 38 | 36 |
| | 0.01 | 0.05 | <0.015 | 5 | <3 |
| D5 | 100 | 0.07 | 0.045 | <3 | <3 |
| | 1 | <0.015 | <0.015 | <3 | 7 |
| | 0.01 | <0.015 | <0.015 | <3 | <3 |

B

IL-6 (pg/ml)

| Positive control | Exp 1 | Exp 2 |
|---|---|---|
| E. coli LPS 10 ng/ml | 14000 | 7300 |
| RPMI media (background) | 12 | 9 |

Most antifungal agents interact with or inhibit synthesis of ergosterol, the major sterol in the fungal plasma membrane (99,109). The polyene antibiotics, such as Amphotericin B, which is often used to treat invasive fungal infections, bind to the membrane ergosterol, causing membrane leakage and cell death, whereas the azole derivatives affect ergosterol biosynthesis (99). Overall, since ergosterol is a key target for most antifungal drugs, their toxicity in mammalian cells would be limited considerably. However, for the membrane-permeabilizing peptides, their interaction with the cell membrane is non-specific, and ergosterol is not uniquely targeted by antimicrobial peptides. Zwitterionic phosphatidylcholine (PC) and phosphatidylethanolamine (PE) are the major phospholipid classes in fungi, with smaller amounts of negatively charged phosphatidylinositol (PI, 3-10%), phosphatidylserine (PS) and diphosphatidylglycerol (DPG, 2-5%) (110). Compared to hRBC (111), fungi have a higher amount of negatively charged PI and DPG. Such differences may result in higher susceptibility of fungal cells to antimicrobial peptides than red blood cells.

The cell wall or cell envelope is a barrier which can hinder AMPs from reaching the cell membrane. Once close to the microbial surface, AMPs must traverse capsular polysaccharides (LPS) and outer membrane components before they can interact with the inner membrane of gram-negative bacteria; on the other hand, AMPs have to traverse capsular polysaccharides, teichoic acids and lipoteichoic acids in order to interact with the membrane of gram-positive bacteria (82). The fungal cell wall is primarily composed of chitin, glucans, mannans and glycoproteins; there is evidence of extensive cross-linking between these components (112). Thus, the fungal cell wall is an even greater barrier to AMPs than the bacterial cell envelope. According to our previous results (93), if the self-association ability of a peptide in aqueous media is too strong (e.g., forming stable folded dimers), it could decrease the ability of the peptide to dissociate and pass through the capsule and cell wall of microorganisms and, hence, prevent penetration into the cytoplasmic membrane to kill target cells. In our current experiments, peptide D4, which has the highest self-association ability (Table 6, FIG. 6), overall exhibits the lowest antimicrobial activity.

FIGS. 6-9 show the relationships between peptide hydrophobicity and antimicrobial and hemolytic activity. Different microorganisms and different strains of the same organism have different responses to increasing peptide hydrophobicity. Clearly, increasing hydrophobicity has the most dramatic effect on eukaryotic cells (as measured by hemolytic activity) as compared to prokaryotic cells. By increasing the peptide hydrophobicity from D1 to D4, hemolytic activity increased 286-fold. In the case of P. aeruginosa, increasing hydrophobicity from L1 to L2 resulted in a 3-fold increase in anti-Pseudomonas activity (FIG. 7). However, a continuing increase in hydrophobicity from L2 to L4 resulted in a dramatic decrease (32-fold) in anti-Pseudomonas activity due to increased peptide self-association (FIG. 7). In fact, L4 was essentially inactive with an MIC value of 500 μg/ml. Although the same trend of decreasing activity with increasing hydrophobicity over and above that of D1 was observed for other gram-negative bacteria (FIG. 8, Panel A), the magnitude of this effect was at least 10-fold smaller compared to the Pseudomonas aeruginosa results (FIG. 7). A similar trend was also observed for gram-positive bacteria (FIG. 8, Panel B), with the magnitude of this effect being similar to the gram negative organisms (FIG. 8, Panel A).

In the case of Zygomycota fungi, decreasing activity with increasing peptide hydrophobicity was also observed for the most hydrophobic peptide, D4 (FIG. 9, Panel A). Thus, in general, increasing hydrophobicity beyond a critical point has a negative impact on antimicrobial activity which can best be explained by peptide self-association. The only exception that we observed was with Ascomycota fungi, where increasing peptide hydrophobicity to D4 (SEQ ID NO:55) resulted in improved activity (FIG. 9).

Overall, when taking into account gram-negative bacteria, gram-positive bacteria and fungi, D1 (SEQ ID NO:24) is the best compound in terms of therapeutic index. However, in the case of Ascomycota fungi, D1 was 5-fold less active than D4 (SEQ ID NO:55) (Table 8). This led us to the challenge of maintaining the activity of D4 for these fungi while increasing the therapeutic index by decreasing the hemolytic activity. D5, with its Lys residue in place of Val in the center of the non-polar face (SEQ. ID NO:56) was 16-fold more active than D4 for Zygomycota fungi, and similar to D4 for Ascomycota fungi, but it had the advantage of a 200-fold improvement in therapeutic index for Zygomycota fungi and an 11-fold improvement for Ascomycota fungi.

Peptide Synthesis and Purification—Syntheses of the peptides were carried out by solid-phase peptide synthesis using t-butyloxycarbonyl chemistry and MBHA (4-methylbenzhydrylamine) resin (0.97 mmol/g), followed by cleavage of the peptide from the resin as described previously (117-119). However, it is understood in the art that there are other suitable instruments and methods for automated or manual peptide synthesis that could be employed to produce the peptides described herein. Peptide purification was performed by reversed-phase high-performance liquid chromatography (RP-HPLC) on a Zorbax 300 SB-C₈ column (250×9.4 mm I.D.; 6.5 μm particle size, 300 Å pore size; Agilent Technologies, Little Falls, Del.) with a linear AB gradient (0.1% acetonitrile/min) at a flow rate of 2 mL/min, where eluent A was 0.2% aqueous trifluoroacetic acid (TFA), pH 2, and eluent B was 0.2% TFA in acetonitrile, where the shallow 0.1% acetonitrile/min gradient started 12% below the acetonitrile concentration required to elute the peptide on injection of analytical sample using a gradient of 1% acetonitrile/min (113). The purity of the peptides was verified by analytical RP-HPLC as described below and further characterized by mass spectrometry and amino acid analysis. Crude and purified peptides were analyzed on an Agilent 1100 series liquid chromatograph. Runs were performed on a Zorbax 300 SB-C8 column (150×2.1 mm I.D.; 5 μm particle size, 300 Å pore size) from Agilent Technologies using a linear AB gradient (1% acetonitrile/min) and a flow rate of 0.25 mL/min, where eluent A was 0.2% aqueous TFA, pH 2, and eluent B was 0.2% TFA in acetonitrile.

### Peptide Killing Assay and Measurement of Anti-Tuberculosis Activity (MIC)

Mycobacterium tuberculosis strain H37Rv was used as a representative mycobacterial strain. Cultures were grown in 71-19 broth for 7-10 days and then diluted to an optical density of McFarland Standard No. 1. This density of cells is approximately 10⁸/ml. The bacterial suspension was then preserved in 1 ml aliquots at ±70° C. until the time of assay. In certain experiments multiple drug resistant M. tuberculosis strain vertulo was used for determination of sensitivity to the D5 peptide.

A fresh suspension of 10⁶ bacteria/ml was made from the frozen stock in Middlebrook 7H9 (Becton Dickinson, Franklin Lakes, N.J.) liquid medium into 5 ml polypropylene tubes (Becton Dickinson). To the fresh bacterial suspension the peptides were added at the desired concentration and incubated for 7 days at 37° C. and 5% CO₂. Samples were plated on Middlebrook 7H11 (Hardy Diagnostics Santa Maria, Calif.) whole plates on day 0 and day 7. The plates were incubated for 3 weeks at 37° C. before counting to determine colony-forming units (CFU)/ml. On the concentration-response format (FIG. 2), the point at which the curve crossed the concentration of the initial inoculum (dashed line) was reported as the minimal inhibitory concentration (MIC). MIC is given as mean value of 4 sets of determinations.

### Measurement of Hemolytic Activity (MHC)

For Protocol A, peptide samples were added to 1% human erythrocytes in phosphate buffered saline (0.08M NaCl; 0.043M Na₂PO₄; 0.011M KH₂PO₄) and reactions were incubated at 37° C. for 12 hours in microtiter plates. Peptide samples were diluted 2 fold in order to determine the concentration that produced no hemolysis. This determination was made by withdrawing aliquots from the hemolysis assays, removing unlysed erythrocytes by centrifugation (800 g) and determining which concentration of peptide failed to cause the release of hemoglobin. Hemoglobin release was determined spectrophotometrically at 562 nm. The hemolytic titer was the highest 2-fold dilution of the peptide that still caused release of hemoglobin from erythrocytes. The control for no release of hemoglobin was a sample of 1% erythrocytes without any peptide added.

Peptide samples were added to 1% human erythrocytes in phosphate-buffered saline (100 mM NaCl, 80 mM Na₂HPO₄, 20 mM NaH₂PO₄, pH 7.4), and the reaction mixtures were incubated at 37° C. for 18 h in microtiter plates. Serial twofold serial dilutions of the peptide samples were carried out in order to determine the concentration that produced no hemolysis. This determination was made by withdrawing aliquots from the hemolysis assays and removing unlysed erythrocytes by centrifugation (800×g). Hemoglobin release was determined spectrophotometrically at 570 nm. The hemolytic activity was determined as the maximal peptide concentration that caused no hemolysis of erythrocytes after 18 h. The control for no release of hemoglobin was a sample of 1% erythrocytes without any peptide added. MHC₅₀ was determined by plot the concentration-lysis format.

In some experiments the hemolytic titer was determined as the highest 2-fold dilution of peptide that caused hemoglobin release. The control for no release of hemoglobin was a sample of 1% erythrocytes without any peptide added. Since erythrocytes were in an isotonic medium, no detectable release (<1% of that released upon complete hemolysis) of hemoglobin was observed from this control during the course of the assay. For the hemolysis time study, hemolytic activity of peptides at concentrations of 8, 16, 32, 64, 125, 250 and 500 μg/ml was measured at 0, 1, 2, 4, 8 hours at 37° C.

### Calculation of Therapeutic Index (MHC₅₀/MIC Ratio)

The therapeutic index is a widely accepted parameter to describe the specificity of antimicrobial reagents. It is calculated by the ratio of MHC₅₀ (hemolytic activity) to MIC (anti-tuberculosis activity); thus, larger values of therapeutic index indicate greater anti-tuberculosis specificity as compared to toxic effects on patient cells.

Both MHC and MIC values were determined by serial 2-fold dilutions. Thus, for individual bacteria and individual peptides the therapeutic index (MHC/MIC, “TI”) could vary by as much as 4 fold if the peptide is very active in both hemolytic and antimicrobial activities; if a peptide has poor or no hemolytic activity, the major variation in the therapeutic index (MHC/MIC) comes from the variation in the MIC value (as much as 2-fold).

Temperature profiling analyses were performed on the same column in 3° C. increments, from 5° C. to 80° C. using a linear AB gradient of 0.5% acetonitrile/min, as described previously (30, 117-119).

### Characterization of Helical Structure

The mean residue molar ellipticities of peptides were determined by circular dichroism (CD) spectroscopy, using a Jasco J-810 spectropolarimeter (Easton, Md.) at 5° C. under benign (non-denaturing) conditions (50 mM NaH₂PO₄/Na₂HPO₄/100 mM KCl, pH 7.0), hereafter referred to as benign buffer, as well as in the presence of an α-helix inducing solvent, 2,2,2-trifluoroethanol, TFE, (50 mM NaH₂PO₄/Na₂HPO₄/00 mM KCl, pH 7.0 buffer/50% TFE). A 10-fold dilution of an approximately 500 M stock solution of the peptide analogs was loaded into a 0.1 cm quartz cell and its ellipticity scanned from 195 to 250 nm. The values of molar ellipticities of the peptide analogs at a wavelength of 222 nm were used to estimate the relative α-helicity of the peptides.

### Determination of Peptide Amphipathicity

Amphipathicity of peptide analogs was determined by the calculation of hydrophobic moment (32) using the software package Jemboss version 1.2.1(33), modified to include a hydrophobicity scale described previously (54). The hydrophobicity scale used in this study is as follows: Trp, 33.0; Phe, 30.1; Leu, 24.6; Ile, 22.8; Met, 17.3; Tyr, 16.0; Val, 15.0; Pro, 10.4; Cys, 9.1; His, 4.7; Ala, 4.1; Arg, 4.1; Thr, 4.1; Gln, 1.6; Ser, 1.2; Asn, 1.0; Gly, 0.0; Glu, −0.4; Asp, −0.8; and Lys, −2.0 (18). These hydrophobicity coefficients were determined from RP-HPLC at pH 7 (10 mM Na₂HPO₄ buffer containing 50 mM NaCl) of a model random coil 10-residue peptide sequence, Ac-X-G-A-K-G-A-G-V-G-L-amide, where position X was substituted by all 20 naturally occurring amino acids (SEQ ID NO:63). This HPLC-derived scale reflects the relative differences in hydrophilicity/hydrophobicity of the 20 amino acid side-chains more accurately than previously determined scales because the substitution site is unaffected by nearest-neighbor or conformational effects (54).

In certain experiments the following hydrophobicity scale was used: Trp, 32.31; Phe, 29.11; Leu, 23.42; Ile 21.31; Met, 16.13; Tyr, 15.37; Val, 13.81; Pro, 9.38; Cys, 8.14; Ala, 3.60; Glu, 3.60; Thr, 2.82; Asp, 2.22; Gln, 0.54; Ser, 0.00; Asn, 0.00; Gly, 0.00; Arg, −5.01; His, −7.03; Lys, −7.03. These hydrophobicity coefficients were determined from reversed-phase chromatography at pH 2 of a model random coil peptide with single substitution of all 20 naturally occurring amino acids. In this case, the amphipathicity is valid for neutral and acidic pH since V₆₈₁ and analogs do not have Asp and Glu residues in their sequences. We propose that this HPLC-derived scale reflects the relative differences in hydrophilicity/hydrophobicity of the 20 amino acid side-chains more accurately than previously determined scales.

### Fungal Strains

The filamentous fungal and yeast strains used in this study were either purchased from American Type Culture Collection, Manassas, Va. (ATCC) or were generous gifts from various institutions: Aspergillus nidulans (AZN 2867), Absidia corymbifera (clinical isolate), Rhizomucor spp. (clinical isolate), Rhizopus microsporus (clinical isolate), Rhizopus oryzae (AZN 8892), Scedosporium prolificans (clinical isolate), Candida albicans (ATCC 24433).

### Measurement of Antifungal Activity (MIC₅₀ and MIC₉₀)

Fungal spores (final concentration 10⁴ spores/ml) were suspended in ½ Potato Dextrose Broth (Difco), and the yeast strains were suspended at a starting A₆₀₀=0.001 in the yeast complete medium YPG (1% yeast extract, 1% peptone, 2% glucose). The medium was supplemented with tetracycline (10 μg/ml) and cefotaxim (100 μg/ml), and dispensed by aliquots of 80 μl into wells of a microplate containing 20 μl of either water or the sample to be analyzed. Growth of fungi and yeasts was evaluated after 24 h at 30° C. by light microscopy and after 48 h by measuring the culture absorbance at 595 nm using a microplate reader. Under conditions where the antifungal assay was performed in the presence of salt, the ½ Potato Dextrose Broth medium was prepared in phosphate-buffered saline, 137 mM NaCl.

The procedure used for the determination of the minimal inhibitory concentration (MIC) was identical to that for the antifungal assay. The MIC values are expressed as the lowest peptide concentration that causes 90% or 50% growth inhibition. The fungicidal effects of the synthetic peptides in the MIC assay were verified by reinoculation of the yeasts in potato dextrose broth at the end of the incubation time.

### Measurement of Antibacterial Activity (MIC)

MICs were determined by a standard microtiter dilution method in Mueller Hinton Broth (MHB). Serial dilutions of the 10× compound were added to the microtiter plates in a volume of 10 μL followed by 90 μL of bacteria for an inoculum of 5×10⁵ colony-forming units (CFU)/mL. The plates were incubated at 37° C. for 24 h, and the MICs were determined as the lowest peptide concentration that inhibited growth.

MICs were determined for certain microorganisms using a standard microtiter dilution method in LB (Luria-Bertani) no-salt broth (10 g tryptone, 5 g yeast extract per liter). Briefly, cells were grown overnight at 37° C. in LB and diluted in the same medium. Serial dilutions of the peptides were added to the microtiter plates in a volume of 100 μl followed by 10 μl of bacteria for an initial concentration of 5×10⁵ CFU/ml. Plates were incubated at 37° C. for 24 hours and MICs determined as the lowest peptide concentration that inhibited growth.

Alternatively; minimal inhibitory concentrations were determined using a standard microtiter dilution method in a Mueller-Hinton (MH) medium. Briefly, cells were grown overnight at 37° C. in MH broth and diluted in the same medium. Serial dilutions of the peptides were added to the microtiter plates in a volume of 100 μl followed by 10 μl of bacteria for an initial cell concentration of 1×10⁵ CFU/ml. Plates were incubated at 37° C. for 24 hours and MICs determined as the lowest peptide concentration that inhibited growth. However, for MIC determination of Pseudomonas aeruginosa clinical isolates, brain heart infusion (BH1) medium was used instead of MH broth and the bacteria were diluted to an initial cell concentration of 1×10⁶ CFU/ml in the test medium.

### Stimulation of Peripheral Blood Mononuclear Cells

Isolation of peripheral blood mononuclear cells (PBMCs) from 5 healthy individuals was performed as described elsewhere (108). Briefly, venous blood was drawn into 10 ml tubes containing 0.2 mg of EDTA (Monoject's-Hertogenbosch, NL). The PBMC fraction was obtained by density centrifugation of blood using Ficoll-Paque (Pharmacia Biotech AB, Sweden). The PBMCs were washed twice in saline and resuspended in culture medium (RPMI 1640 Dutch modification, ICN Biomedicals, Costa Mesa, Calif.), supplemented with gentamicin 1%, L-glutamine 1% and pyruvate 1%. The PBMCs were incubated in 96-well tissue culture plates (Greiner, Alphen, NL) at a concentration of 5×10⁵ cells per well in a total volume of 200 μl, in the presence or absence of a set of stimuli in different experiments. These stimuli consisted of a three concentration dose-response range of the various peptides (0.01, 1.0 and 100 μg/ml). After 24 h of incubation, the supernatants were collected and stored at −80° C. until analysis.

### Cytokine Measurements

Interleukin-6 (IL-6) and tumor necrosis factor (TNF) were measured by ELISA according to the manufacturer's protocol (Pelikine, CLB, Amsterdam, NL). The crude peptides were purified by preparative reversed-phase chromatography (RP-HPLC) using a Zorbax 300 SB-C₈ column (250×9.4 mm I.D.; 6.5 μm particle size, 300 Å pore size; Agilent Technologies) with a linear AB gradient (0.2% acetonitrile/min) at a flow rate of 2 ml/min, where mobile phase A was 0.1% aqueous TFA in water and B was 0.1% TFA in acetonitrile. The purity of peptides was verified by analytical RP-HPLC. The peptides were further characterized by electrospray mass spectrometry and amino acid analysis.

Analytical RP-HPLC of Peptides—Peptides were analyzed on an Agilent 1100 series liquid chromatograph (Little Falls, Del.). Runs were performed on a Zorbax 300 SB-C₈ column (150×2.1 mm I.D.; 5 μm particle size, 300 Å pore size) from Agilent Technologies using linear AB gradient (1% acetonitrile/min) and a flow rate of 0.25 ml/min, where solvent A was 0.05% aqueous TFA, pH 2 and solvent B was 0.05% TFA in acetonitrile. Temperature profiling analyses were performed in 3° C. increments, from 5° C. to 80° C.

CD Temperature Denaturation Study of Peptide V₆₈₁—The native peptide V₆₈₁ was dissolved in 0.05% aqueous TFA containing 50% TFE, pH 2, loaded into a 0.02 cm fused silica cell and peptide ellipticity scanned from 190 to 250 nm at temperatures of 5, 15, 25, 35, 45, 55, 65 and 80° C. The spectra at different temperatures were used to mimic the alteration of peptide conformation during temperature profiling analysis in RP-HPLC. The ratio of the molar ellipticity at a particular temperature (t) relative to that at 5° C. ([θ]ₜ−[θ]ᵤ)/([θ]₅−[θ]ᵤ) was calculated and plotted against temperature in order to obtain the thermal melting profiles, where [θ]₅ and [θ]ᵤ represent the molar ellipticity values for the fully folded and fully unfolded species, respectively. [O] was determined in the presence of 8M urea with a value of 1500 deg·cm²·dmol⁻¹ to represent a totally random coil state (31). The melting temperature (Tₘ) was calculated as the temperature at which the α-helix was 50% denatured (([θ]ₜ−[θ]ᵤ)/([θ]₅−θ[θ]ᵤ)=0.5) and the values were taken as a measure of α-helix stability.

Proteolytic stability assay—Proteolytic stability of the peptides was carried out with trypsin in a molar ratio of 1:20,000 (trypsin:peptide=0.1 μM:2 mM). The buffer used was 50 mM NH₄HCO₃ at pH 7.4 for both peptides and enzyme. The mixtures of peptide and trypsin were incubated at 37° C. Samples were collected at time points of 0, 5 min, 10 min, 20 min, 30 min, 1, 2, 4, 8 hours. Equal volumes of 20% aqueous TFA were added to each sample to stop the reaction and peptide degradation was checked by RP-HPLC. Runs were performed on a Zorbax 300 SB-C₈ column (150×2.1 mm I.D.; 5 μm particle size, 300 Å pore size) from Agilent Technologies at room temperature using a linear AB gradient (1% acetonitrile/min) and a flow rate of 0.25 ml/min, where eluent A was 0.2% aqueous TFA, pH 2 and eluent B was 0.2% TFA in acetonitrile. The change in integrated peak area of the peptides was used to monitor the degree of proteolysis during the time study.

### Example 2

### Peptide Analogs with Varied Position of Substitution

The correlation between peptide hydrophobicity and hemolytic activity can be explained by the “membrane discrimination” mechanism. Peptides with higher hydrophobicity penetrate deeper into the hydrophobic core of red blood cell membrane (67), causing stronger hemolysis by forming pores or channels, i.e., A12L/A23L (peptide 5) and A12L/A20L (peptide 6) exhibited stronger hemolytic activity than single Leu-substituted peptides, and A12UA20L/A23L (peptide 7) showed the strongest hemolytic activity in this study. For peptide antimicrobial activity, since the insertion of the molecules into the hydrophobic core is not necessary to lyse bacterial cells during the antibacterial action, peptides only lie at the interface parallel with the membrane allowing their hydrophobic surface to interact with the hydrophobic component of the lipid, and the positive charge residues to interact with the negatively charged head groups of the phospholipids (46,47). Thus, it is reasonable to assume that increasing peptide hydrophobicity to a certain extent will help peptide molecules to reach the interface from aqueous environment and improve antimicrobial activity. In this study, the improvement of antimicrobial activity from peptide NK_{L} (peptide 1) to peptide A20L (peptide 4) can represent such an advantage of increasing hydrophobicity. In contrast, further increases in hydrophobicity will cause the stronger peptide dimerization in solution which in turn results in the monomer-dimer equilibrium favoring the dimer conformation. Peptide dimers are in their folded α-helical conformation and would be inhibited from passing through the cell wall to reach the target membranes. Hence the antimicrobial activities of peptides A12L/A23L (peptide 5) and A12L/A20L (peptide 6) become weaker with increasing hydrophobicity compared to the single Leu-substituted analogs. We believe that there is a threshold of hydrophobicity controlling peptide antimicrobial activity, that is, one may adjust peptide hydrophobicity to obtain the optimal antimicrobial activity. For the extreme example of the triple-Leu-substituted analog, A12L/A20L/A23L (peptide 7), the loss of antimicrobial activity may be explained as due to its very strong dimerization ability in aqueous environments. Hence, the peptide exists mainly as a dimer in solution and it would not pass through the bacterial cell wall. In contrast, there is no polysaccharide-based cell wall in eukaryotic cells, thus, A12L/A20L/A23L (peptide 7) caused severe hemolysis against human red blood cells where the hydrophobicity of the bilayer causes rapid dissociation of dimers to monomers and entry into the bilayer to form channels/pores.

### Example 3

### Peptide Analogs with Varied Nature of Charge Substitution

Further peptides of the invention are generated by varying the nature of the charged residue selected for the substitution. In the context of D5 (SEQ ID NO:56), for example, the position for substitution is established as position 13. The amino acid selected for substitution is preferably a charged amino acid and is in particular an amino acid with a net positive charge. Particular examples of positively charged (basic) residues at positions 13 and 16 are Lys, Arg, Orn, H is, diaminobutyric acid and diaminopropionic acid. We note that Orn has a delta-amino group instead of an epsilon/□-amino group in Lys, i.e., the side-chain is shorter by one carbon atom; diaminobutyric acid is one carbon shorter than Orn; i.e., it has a gamma-amino group; diaminopropionic acid is two carbons shorter than Orn.

### Example 4

### Truncated Peptide Analogs

Further peptides of the invention are generated by truncation of a reference peptide such as SEQ ID NO:56 or a peptide of the invention or any of SEQ ID NOS: 53 to 62. For example, truncation of the N-terminal residue Lys1 or C-terminal residues Ser25 and Ser26 does not substantially affect the biological properties such as antimicrobial activity of the truncated peptide. It is believed, however, that truncation of Lys1 and Trp2 can substantially decrease the therapeutic index due to removal of the large hydrophobe, Trp. Similarly, truncation of Ser26, Ser25 and Ile24 can substantially decrease the therapeutic index due to removal of the large hydrophobe, Ile.

### Example 5

### Shuffled Peptide Analogs

Peptides are generated having a range of overall hydrophobicity of the non-polar face. The hydrophobicity of the non-polar face can be calculated using a sum of the hydrophobicity coefficients listed herein. For example, a particular hydrophobicity range is of NK_{L} or NA_{D}±the value of a Leu side-chain. Using our scale, the hydrophobicity of the non-polar face of NK_{L} sums up the values for W2, F5, L6, F9, A12, K13, V16, L17, A20, L21, A23, I24 getting a value of 199.7. See below.

TABLE 10
Hydrophobicity coefficients.

| Item | Coefficient |
|---|---|
| Trp 2 | 32.31 |
| Phe 5 | 29.11 |
| Leu 6 | 23.42 |
| Phe 9 | 29.11 |
| Ala 12 | 3.60 |
| Lys 13 | −7.03 |
| Val 16 | 13.81 |
| Leu 17 | 23.42 |
| Ala 20 | 3.60 |
| Leu 21 | 23.42 |
| Ala 23 | 3.60 |
| Ile 24 | 21.31 |
| SUM | 199.7 ± 23.42 |

Different scales can give different values. For certain peptides specifically set forth herein, there is significance in the sum of the residues in the hydrophobic surface, using our scale, where the surface hydrophobicity range that generates the desired biological activity is from about 176 to about 224.

The sum of the hydrophobicity coefficients for the polar face should be the value for NK_{L} peptide ±the value of a Lys residue.

TABLE 11
Coefficient values.

| Item | Coefficient |
|---|---|
| K1 | −7.03 |
| K3 | −7.03 |
| S4 | 0.00 |
| K7 | −7.03 |
| T6 | +2.82 |
| K10 | −7.03 |
| S11 | 0.00 |
| K14 | −7.03 |
| T15 | +2.82 |
| H18 | −7.03 |
| T19 | +2.82 |
| K22 | −7.03 |
| S25 | 0.00 |
| S26 | 0.00 |
| SUM | −40.75 ± 7.03 |

Using our scale, the hydrophobicity of the polar face of NK_{L} sums up the values K1, K3, S4, K7, T6, K10, S11, K14, T15, H18, T19, K22, S25 and S26. The range of surface hydrophilicity that generates the desired biological activity is from about −33 to about −48.

### Example 6

### Peptide Analogs with Similar Single Hydrophobicity Substitutions

Further peptides of the invention are generated by making single substitutions of amino acid residues with relatively similar hydrophobicity. Single hydrophobicity substitutions with side-chains of similar hydrophobicity are generated and have biological activity. For example, possible substitutions for each residue in the non-polar face are listed below in the context of peptides D1 to D10 (SEQ ID NOS:24 and 53-62).

Residues for single substitutions can be as follows: Ile, Val, norleucine, norvaline for Leu; Leu, Val, norleucine, norvaline for Ile; Leu, Ile, norleucine, norvaline for Val; Leu, Ile, Val, norleucine, norvaline for Phe; and Phe, Leu, Ile, Val, norleucine, norvaline for Trp.

All references (patent and non-patent literature or other source material) cited throughout this application are hereby incorporated by reference herein in their entireties, as though individually incorporated by reference, to the extent each reference is not inconsistent with the present disclosure in this application. References cited herein reflect the level of skill in the relevant arts.

The Sequence Listing provided herewith is incorporated by reference herein.

Where the terms “comprise”, “comprises”, “comprised”, or “comprising” are used herein, they are to be interpreted as specifying the presence of the stated features, integers, steps, or components referred to, but not to preclude the presence or addition of one or more other feature, integer, step, component, or group thereof.

The invention has been described with reference to various specific and preferred embodiments and techniques. However, it should be understood that many variations and modifications may be made while remaining within the true spirit and scope of the invention. It will be apparent to one of ordinary skill in the art that compositions, methods and materials, other than those specifically described herein can be applied to the practice of the invention as broadly disclosed herein without resort to undue experimentation. All art-known functional equivalents of compositions, methods and materials described herein are intended to be encompassed by this invention. It is not intended, however, for any claim herein to specifically encompass any precise embodiment existing and legally qualifying in the relevant jurisdiction as prior art for novelty; a claim purportedly encompassing such an embodiment is intended to be of scope so as to just exclude any such precise embodiment.

Whenever a range is disclosed, all subranges and individual values are intended to be encompassed. This invention is not to be limited by the embodiments disclosed, including any shown in the drawings or exemplified in the specification, which are given by way of example or illustration and not of limitation.

For certain α-helical and β-sheet peptides, attempts have been made to delineate features responsible for anti-eukaryotic or toxic activities and/or for antimicrobial activities. High amphipathicity (17-20), high hydrophobicity (17,20-22), as well as high helicity or β-sheet structure (20,23,24) may correlate with increased toxicity as measured by hemolytic activity. In contrast, antimicrobial activity may be less dependent on these factors than is hemolytic activity (17-21,23-25). Specificity (or therapeutic index, TI, which is defined as the ratio of hemolytic activity to antimicrobial activity for a bacterium or fungus of interest) could be increased in one of three ways: increasing antimicrobial activity, decreasing hemolytic activity while maintaining antimicrobial activity, or simultaneously increasing antimicrobial activity and decreasing hemolytic activity.

### REFERENCES

1. Neu, H. C. (1992) Science 257, 1064-1073
2. Travis, J. (1994) Science 264, 360-362
3. Hancock, R. E. (1997) Lancet 349, 418-422
4. Andreu, D., and Rivas, L. (1998) Biopolymers 47, 415-433
5. Sitaram, N., and Nagaraj, R. (2002) Curr Pharm Des 8, 727-742
6. Hancock, R. E., and Lehrer, R. (1998) Trends Biotechnol 16, 82-88
7. Duclohier, H., Molle, G., and Spach, G. (1989) Biophys J 56, 1017-1021
8. van't H of, W., Veerman, E. C., Helmerhorst, E. J., and Amerongen, A. V. (2001) Biol Chem 382, 597-619
9. Devine, D. A., and Hancock, R. E. (2002) Curr Pharm Des 8, 703-714
10. Ganz, T., and Lehrer, R. I. (1994) Curr Opin Immunol 6, 584-589
11. Steinberg, D. A., Hurst, M. A., Fujii, C. A., Kung, A. H., Ho, J. F., Cheng, F. C., Loury, D. J., and Fiddes, J. C. (1997) Antimicrob Agents Chemother 41, 1738-1742
12. Khaled, M. A., Urry, D. W., Sugano, H., Miyoshi, M., and lzumiya, N. (1978) Biochemistry 17, 2490-2494
13. Mootz, H. D., and Marahiel, M. A. (1997) J Bacteriol 179, 6843-6850
14. Christensen, B., Fink, J., Merrifield, R. B., and Mauzerall, D. (1988) Proc Natl Acad Sci USA 85, 5072-5076
15. Zasloff, M. (1987) Proc Natl Acad Sci USA 84, 5449-5453
16. Andreu, D., Ubach, J., Boman, A., Wahlin, B., Wade, D., Merrifield, R. B., and Boman, H. G. (1992) FEBS Lett 296, 190-194
17. Dathe, M., Wieprecht, T., Nikolenko, H., Handel, L., Maloy, W. L., MacDonald, D. L., Beyermann, M., and Bienert, M. (1997) FEBS Lett 403, 208-212
18. Blondelle, S. E., and Houghten, R. A. (1992) Biochemistry 31, 12688-12694
19. Lee, D. L., and Hodges, R. S. (2003) Biopolymers 71, 28-48
20. Kondejewski, L. H., Jelokhani-Niaraki, M., Farmer, S. W., Lix, B., Kay, C. M., Sykes, B. D., Hancock, R. E., and Hodges, R. S. (1999) J Biol Chem 274, 13181-13192
21. Oren, Z., Hong, J., and Shai, Y. (1997) J Biol Chem 272, 14643-14649
22. Kondejewski, L. H., Lee, D. L., Jelokhani-Niaraki, M., Farmer, S. W., Hancock, R. E., and Hodges, R. S. (2002) J Biol Chem 277, 67-74
23. Shai, Y., and Oren, Z. (1996) J Biol Chem 271, 7305-7308
24. Oren, Z., and Shai, Y. (1997) Biochemistry 36, 1826-1835
25. Lee, D. L., Powers, J. P., Pflegerl, K., Vasil, M. L., Hancock, R. E., and Hodges, R. S. (2004) J Pept Res 63, 69-84
26. Chen, Y., Mant, C. T., and Hodges, R. S. (2002) J Pept Res 59, 18-33
27. Zhang, L., Falla, T., Wu, M., Fidai, S., Burian, J., Kay, W., and Hancock, R. E. (1998) Biochem Biophys Res Commun 247, 674-680
28. Zhang, L., Benz, R., and Hancock, R. E. (1999) Biochemistry 38, 8102-8111
29. Mant, C. T., Chen, Y., and Hodges, R. S. (2003) J Chromatogr A 1009, 29-43
30. Lee, D. L., Mant, C. T., and Hodges, R. S. (2003) J Biol Chem 278, 22918-22927
31. Monera, O. D., Sereda, T. J., Zhou, N. E., Kay, C. M., and Hodges, R. S. (1995) Journal of Peptide Science 1, 319-329
32. Eisenberg, D., Weiss, R. M., and Terwilliger, T. C. (1982) Nature 299, 371-374
33. Carver, T., and Bleasby, A. (2003) Bioinformatics 19, 1837-1843
34. Zhou, N. E., Monera, O. D., Kay, C. M., and Hodges, R. S. (1994) Protein Peptide Lett. 1, 114-119
35. McInnes, C., Kondejewski, L. H., Hodges, R. S., and Sykes, B. D. (2000) J Biol Chem 275, 14287-14294
36. Mant, C. T., Zhou, N. E., and Hodges, R. S. (1993) in The Amphipathic Helix (Epand, R. M., ed), pp. 39-64, CRC Press, Boca Raton
37. Mant, C. T., and Hodges, R. S. (2002) J Chromatogr A 972, 61-75
38. Mant, C. T., and Hodges, R. S. (2002) J Chromatogr A 972, 45-60
39. Zhou, N. E., Mant, C. T., and Hodges, R. S. (1990) Pept Res 3, 8-20
40. Blondelle, S. E., Ostresh, J. M., Houghten, R. A., and Perez-Paya, E. (1995) Biophys J 68, 351-359
41. Purcell, A. W., Aguilar, M. I., Wettenhall, R. E., and Hearn, M. T. (1995) Pept Res 8, 160-170
42. Mant, C. T., Tripet, B., and Hodges, R. S. (2003) J Chromatogr A 1009, 45-59
43. Mant, C. T., and Hodges, R. S. (eds) (1991) HPLC of peptides and proteins: separation, analysis and conformation, CRC Press, Boca Raton, Fla.
44. Shai, Y. (1999) Biochim Biophys Acta 1462, 55-70
45. Ehrenstein, G., and Lecar, H. (1977) Q Rev Biophys 10, 1-34
46. Pouny, Y., Rapaport, D., Mor, A., Nicolas, P., and Shai, Y. (1992) Biochemistry 31, 12416-12423
47. Salgado, J., Grage, S. L., Kondejewski, L. H., Hodges, R. S., McElhaney, R. N., and Ulrich, A. S. (2001) J Biomol NMR 21, 191-208
48. Liu, F., Lewis, R. N., Hodges, R. S., and McElhaney, R. N. (2004) Biophysical Journal 87, 2470-2482
49. Liu, F., Lewis, R. N., Hodges, R. S., and McElhaney, R. N. (2004) Biochemistry 43, 3679-368
50. Liu, F., Lewis, R. N., Hodges, R. S., and McElhaney, R. N. (2002) Biochemistry 41, 9197-9207
51. Daum, G. (1985) Biochim Biophys Acta 822, 1-42
52. Devaux, P. F., and Seigneuret, M. (1985) Biochim Biophys Acta 822, 63-125
53. Chen, Y., Mant, C. T., Farmer, S. W., Hancock, R. E., Vasil, M. L. and Hodges, R. S. (2005) Rational design of alpha-helical antimicrobial peptides with enhanced activities and specificity/therapeutic index. J Biol Chem 280: 12316-12329
54. Kovacs, J. M., Mant, C. T. and Hodges, R. S. (2006) Determination of intrinsic hydrophilicity/hydrophobicity of amino acid side-chains in peptides in the absence of nearest-neighbor or conformational effects. Biopolymers (Peptide science) 84, 283-297
55. Dolan, J. W. (2002) Temperature selectivity in reversed-phase high performance liquid chromatography. J Chromatogr A 965: 195-205
56. Powers, J. P., Rozek, A. and Hancock, R. E. (2004) Structure-activity relationships for the beta-hairpin cationic antimicrobial peptide polyphemusin i. Biochim Biophys Acta 1698: 239-250
57. Wade, D., Boman, A., Wahlin, B., Drain, C. M., Andreu, D., Boman, H. G. and Merrifield, R. B. (1990) All-d amino acid-containing channel-forming antibiotic peptides. Proc Natl Acad Sci USA 87: 4761-4765
58. Hoiby, N. and Koch, C. (1990) Cystic fibrosis. 1. Pseudomonas aeruginosa infection in cystic fibrosis and its management. Thorax 45: 881-884
59. Elkin, S. and Geddes, D. (2003) Pseudomonal infection in cystic fibrosis: The battle continues. Expert Rev Anti Infect Ther 1: 609-618
60. Pierce, G. E. (2005) Pseudomonas aeruginosa, candida albicans, and device-related nosocomial infections: Implications, trends, and potential approaches for control. J Ind Microbiol Biotechnol 32: 309-318
64. Avrahami, D. and Shai, Y. (2002) Conjugation of a magainin analogue with lipophilic acids controls hydrophobicity, solution assembly, and cell selectivity. Biochemistry 41: 2254-2263
65. Wieprecht, T., Dathe, M., Beyermann, M., Krause, E., Maloy, W. L., MacDonald, D. L. and Bienert, M. (1997) Peptide hydrophobicity controls the activity and selectivity of magainin 2 amide in interaction with membranes. Biochemistry 36: 6124-6132
66. Kustanovich, I., Shalev, D. E., Mikhlin, M., Gaidukov, L. and Mor, A. (2002) Structural requirements for potent versus selective cytotoxicity for antimicrobial dermaseptin s4 derivatives. J Biol Chem 277: 16941-16951
67. Tachi, T., Epand, R. F., Epand, R. M. and Matsuzaki, K. (2002) Position-dependent hydrophobicity of the antimicrobial magainin peptide affects the mode of peptide-lipid interactions and selective toxicity. Biochemistry 41: 10723-10731
68. Lugtenberg, B. and Van Alphen, L. (1983) Molecular architecture and functioning of the outer membrane of Escherichia coli and other gram-negative bacteria. Biochim Biophys Acta 737: 51-115
69. Zilberstein, D., Schuldiner, S, and Padan, E. (1979) Proton electrochemical gradient in Escherichia coli cells and its relation to active transport of lactose. Biochemistry 18: 669-673
70. Boman, H. G. (2003) Antibacterial peptides: Basic facts and emerging concepts. J Intern Med 254: 197-215
71. Dathe, M., Schumann, M., Wieprecht, T., Winkler, A., Beyermann, M., Krause, E., Matsuzaki, K., Murase, O. and Bienert, M. (1996) Peptide helicity and membrane surface charge modulate the balance of electrostatic and hydrophobic interactions with lipid bilayers and biological membranes. Biochemistry 35: 12612-12622
72. Wieprecht, T., Dathe, M., Krause, E., Beyermann, M., Maloy, W. L., MacDonald, D. L. and Bienert, M. (1997) Modulation of membrane activity of amphipathic, antibacterial peptides by slight modifications of the hydrophobic moment. FEBS Lett 417: 135-140
73. Blondelle, S. E. and Houghten, R. A. (1991) Hemolytic and antimicrobial activities of the twenty-four individual omission analogues of melittin. Biochemistry 30: 4671-4678
74. Kiyota, T., Lee, S, and Sugihara, G. (1996) Design and synthesis of amphiphilic alpha-helical model peptides with systematically varied hydrophobic-hydrophilic balance and their interaction with lipid- and bio-membranes. Biochemistry 35: 13196-13204
75. Blondelle, S. E., Lohner, K. and Aguilar, M. (1999) Lipid-induced conformation and lipid-binding properties of cytolytic and antimicrobial peptides: Determination and biological specificity. Biochim Biophys Acta 1462: 89-108
76. Hancock, R. E. and Rozek, A. (2002) Role of membranes in the activities of antimicrobial cationic peptides. FEMS Microbiol Lett 206: 143-149
77. Sitaram, N. and Nagaraj, R. (1999) Interaction of antimicrobial peptides with biological and model membranes: Structural and charge requirements for activity. Biochim Biophys Acta 1462: 29-54
78. Shai, Y. (1999) Mechanism of the binding, insertion and destabilization of phospholipid bilayer membranes by alpha-helical antimicrobial and cell non-selective membrane-lytic peptides. Biochim Biophys Acta 1462: 55-70
79. Matsuzaki, K. (1999) Why and how are peptide-lipid interactions utilized for self-defense?Magainins and tachyplesins as archetypes. Biochim Biophys Acta 1462: 1-10
80. Chen, Y., Mehok, A. R., Mant, C. T. and Hodges, R. S. (2004) Optimum concentration of trifluoroacetic acid for reversed-phase liquid chromatography of peptides revisited. J Chromatogr A 1043: 9-18
81. Reddy, K. V., Yedery, R. D. and Aranha, C. (2004) Antimicrobial peptides: Premises and promises. Int J Antimicrob Agents 24: 536-547
82. Brogden, K. A. (2005) Antimicrobial peptides: Pore formers or metabolic inhibitors in bacteria? Nat Rev Microbiol 3: 238-250
83. Bland, J. M., De Lucca, A. J., Jacks, T. J. and Vigo, C. B. (2001) All-D-cecropin b: Synthesis, conformation, lipopolysaccharide binding, and antibacterial activity. Mol Cell Biochem 218: 105-111
84. De Lucca, A. J., Bland, J. M., Vigo, C. B., Jacks, T. J., Peter, J. and Walsh, T. J. (2000) D-cecropin b: Proteolytic resistance, lethality for pathogenic fungi and binding properties. Med Mycol 38: 301-308
85. Cribbs, D. H., Pike, C. J., Weinstein, S. L., Velazquez, P. and Cotman, C. W. (1997) All-D-enantiomers of beta-amyloid exhibit similar biological properties to all-L-beta-amyloids. J Biol Chem 272: 7431-7436
86. Hamamoto, K., Kida, Y., Zhang, Y., Shimizu, T. and Kuwano, K. (2002) Antimicrobial activity and stability to proteolysis of small linear cationic peptides with D-amino acid substitutions. Microbiol Immunol 46: 741-749
87. Elmquist, A. and Langel, U. (2003) In vitro uptake and stability study of pVEC and its all-D analog. Biol Chem 384: 387-393
88. Hong, S. Y., Oh, J. E. and Lee, K. H. (1999) Effect of D-amino acid substitution on the stability, the secondary structure, and the activity of membrane-active peptide. Biochem Pharmacol 58: 1775-1780
89. Wakabayashi, H., Matsumoto, H., Hashimoto, K., Teraguchi, S., Takase, M. and Hayasawa, H. (1999) N-acylated and D enantiomer derivatives of a nonamer core peptide of lactoferricin B showing improved antimicrobial activity. Antimicrob Agents Chemother 43: 1267-1269
90. Guo, D., Mant, C. T., Taneja, A. K., Parker, J. M. R. and Hodges, R. S. (1986) Prediction of peptide retention times in reversed-phase high-performance liquid chromatography. I. Determination of retention coefficients of amino acid residues using model synthetic peptides. J Chromatogr 359: 499-518
91. Zhang L, Rozek A, Hancock R E. Interaction of cationic antimicrobial peptides with model membranes. J Biol. Chem. 2001 Sep. 21; 276(38):35714-22.
92. Chen, Y., Vasil, A. I., Rehaume, L., Mant, C. T., Burns, J. L., Vasil, M. L., Hancock, R. E., and Hodges, R. S. (2006) Chem Biol Drug Des 67, 162-173
93. Chen, Y., Guarnieri, M. T., Vasil, A. I., Vasil, M. L., Mant, C. T., and Hodges, R. S. (2007) Antimicrob Agents Chemother 51, 1398-1406
94. Chen, Y., Mant, C. T., and Hodges, R. S. (2007) J Chromatogr A 1140, 112-120
95. Dennison, S. R., Harris, F., and Phoenix, D. A. (2003) Protein Pept Lett 10, 497-502
96. Dennison, S. R., Wallace, J., Harris, F., and Phoenix, D. A. (2005) Protein Pept Lett 12, 31-39
97. Tossi, A., Sandri, L., and Giangaspero, A. (2000) Biopolymers 55, 4-30
98. Baumann, G., and Mueller, P. (1974) J Supramol Struct 2, 538-557
99. Georgopapadakou, N. H., and Walsh, T. J. (1994) Science 264, 371-373
100. Paya, C. V. (1993) Clin Infect Dis 16, 677-688
101. Brown, A. E. (1990) Semin Oncol 17, 2-5
102. Durden, F. M., and Elewski, B. (1997) Semin Cutan Med Surg 16, 200-212
103. Hodges, R. S., Jiang, Z., Whitehurst, J., and Mant, C. T. (in press) Development of antimicrobial peptides as therapeutic agents. In: Development of therapeutic agents, Leventhal, M., and Gad, S. C. (eds). John Wiley & Sons, Inc., Hoboken, N.J.
104. Lustig, F., Hoebeke, J., Ostergren-Lunden, G., Velge-Roussel, F., Bondjers, G., Olsson, U., Ruetschi, U., and Fager, G. (1996) Biochemistry 35, 12077-12085
105. Jenssen, H., Hamill, P., and Hancock, R. E. (2006) Clin Microbiol Rev 19, 491-511
106. Jiang, Z., Vasil, A. I., Hale, J. D., Hancock, R. E., Vasil, M. L., and Hodges, R. S. (2008) Biopolymers (Peptide Science) 90, 369-383
107. Kovacs, J. M., Mant, C. T., and Hodges, R. S. (2006) Biopolymers (Peptide Science) 84, 283-297
108. Drenth, J. P., Van Uum, S. H., Van Deuren, M., Pesman, G. J., Van der Ven-Jongekrijg, J., and Van der Meer, J. W. (1995) J Appl Physiol 79, 1497-1503
109. Kontoyiannis, D. P., and Lewis, R. E. (2002) Lancet 359, 1135-1144
110. Prasad, R., and Ghannoum, M. A. (1996) Lipids of Pathogenic Fungi CRC press
111. Gabriel, G. J., Som, A., Madkour, A. E., Eren, T., and Tew, G. N. (2007) Mater Sci Eng R Rep 57, 28-64
112. Bowman, S. M., and Free, S. J. (2006) Bioessays 28, 799-808
113. Chen, Y., Mant, C. T., and Hodges, R. S. (2007) J Chromatogr A 1140, 112-120
114. Chen, Y., Mant, C. T., and Hodges, R. S. (2002) J Pept Res 59, 18-33
115. Hancock, R. E. (1997) Lancet 349, 418-422
116. Hodges, R. S., Jiang, Z., Whitehurst, J., and Mant, C. T. (2008 in press) Development of antimicrobial peptides as therapeutic agents. In: Development of therapeutic agents, Leventhal, M., and Gad, S. C. (eds). John Wiley & Sons, Inc., Hoboken, N.J.
117. Chen, Y., Mant, C. T., Farmer, S. W., Hancock, R. E., Vasil, M. L., and Hodges, R. S. (2005) J Biol Chem 280, 12316-12329
118. Chen, Y., Vasil, A. I., Rehaume, L., Mant, C. T., Burns, J. L., Vasil, M. L., Hancock, R. E., and Hodges, R. S. (2006) Chem Biol Drug Des 67, 162-173
119. Chen, Y., Guarnieri, M. T., Vasil, A. I., Vasil, M. L., Mant, C. T., and Hodges, R. S. (2007) Antimicrob Agents Chemother 51, 1398-1406
120. Jiang, Z., Vasil, A. I., Hale, J. D., Hancock, R. E., Vasil, M. L., and Hodges, R. S. (2008) Biopolymers (Peptide Science) 90, 369-383
121. Chen, Y., Mant, C. T., and Hodges, R. S. (2007) J Chromatogr A 1140, 112-120
U.S. Pat. Nos. 6,906,035, 6,818,407, 6,747,007, 6,465,429, 6,358,921, 6,337,317, 6,297,215, 6,288,212, 6,191,254, 6,172,185, 6,057,291, 6,040,435, 5,877,274, 5,789,377, 5,707,855, 5,688,767, 5,593,866, 20030228324, 20030021795, 6,872,806.

1. A peptide having antimicrobial activity, said peptide comprising a sequence having a general formula derived from SEQ ID NO:6 and having one or more improved biological properties relative to SEQ ID NO:6, wherein said one or more properties are selected from the group consisting of antimicrobial activity, hemolytic activity, stability, and therapeutic index for a microorganism, with the proviso that the peptide does not have an amino acid sequence set forth in any of SEQ ID NO:1-55, wherein optionally at least one amino acid is a D-amino acid.

2. The peptide of claim 1, wherein the peptide is from about 23 to about 26 amino acids in length.

3. The peptide of claim 2 wherein the peptide is selected from the group consisting of SEQ ID NOS:56, 57, 58, 59, 60, 61 and 62.

4. The peptide of claim 1, wherein the peptide comprises a core sequence of FLKTFKSLKKTKLHTLL (amino acids 5 to 21 of SEQ ID NO:56).

5. The peptide of claim 1, wherein said peptide has the sequence set forth in SEQ ID NO:56.

6. The peptide of claim 5, wherein each amino acid residue is a D-amino acid residue.

7. The peptide of claim 1 with at least one of an optional C-terminal amide, an N-terminal acetylation, or N-polyethylene glycol modification.

8. A therapeutic composition for controlling infection by a microorganism, said composition comprising at least one antimicrobial peptide claim 1 in a therapeutically effective amount and a pharmaceutically acceptable carrier.

9. The composition of claim 8, wherein the peptide comprises the peptide of the sequence as set forth in SEQ ID NO:56.

10. The composition of claim 8, wherein the peptide comprises a core sequence of FLKTFKSLKKTKLHTLL (amino acids 5 to 21 of SEQ ID NO:56), optionally wherein each amino acid residue in the peptide is a D-amino acid residue.

11-14. (canceled)

15. A method of controlling growth of a microorganism, said method comprising the step of administering an effective amount of a composition comprising at least one antimicrobial peptide of claim 1 or
a peptide having the sequence of SEQ ID NO:24 when the microorganism is a fungus.

16. The method of claim 15, wherein said microorganism is a gram-negative bacterium, a gram-positive bacterium, mycobacterium or fungus.

17. The method of claim 15, wherein the microorganism is a fungus and wherein the peptide has a sequence set forth in any one of SEQ ID NOs:52-62.

18. The method of claim 16, wherein the bacterium is a mycobacterium, and wherein the peptide is has the amino acid sequence set forth in SEQ ID NO:24 or the amino acid sequence set forth in SEQ ID NO:24 in which all amino acids are D-amino acids.

19. The method of claim 18, wherein the bacterium is Mycobacterium tuberculosis, optionally, wherein the peptide is that of SEQ ID NO:56 or SEQ ID NO:24.

20. The method of claim 19, wherein the peptide has the sequence of SEQ ID NO:56, and optionally wherein each amino acid residue is a D-amino acid residue.

21. The method of claim 17, wherein the fungus is a Zygomycota fungus and the peptide has the sequence of SEQ ID NO:56.

22. The method of claim 17, wherein the fungus is an Ascomycota fungus and the peptide has the sequence of SEQ ID NO:53, 54, 55, 56, 57, 58, 59, 60, 61 or 62.

23. The method of claim 22 wherein the peptide has the sequence of SEQ ID NO:56.

24. A method of treating a subject infected by a microorganism for which treatment is needed or of reducing the incidence or severity of an infection in a subject caused by a microorganism, wherein said method comprises the step of administering a therapeutically effective amount of a composition to a subject with the infection, said composition comprising at least one antimicrobial peptide of claim 1 and a pharmaceutically acceptable carrier, and optionally including an additional therapeutic agent.

25. The method of claim 24, wherein said antimicrobial peptide is characterized by the sequence set forth as amino acids 5 to 21 of SEQ ID NO:56, or as set forth in SEQ ID NO:56, and wherein at least one or each amino acid residue is a D-amino acid residue.

26. The method of claim 24, wherein the microorganism is a zygomyceta fungus, ascomyceta fungus, gram-positive bacterium, gram-negative bacterium or acid-fast bacterium.

27-30. (canceled)

31. The method of claim 26, wherein the microorganism is a Mycobacterium.

32. The method of claim 31, wherein the microorganism is Mycobacterium tuberculosis and wherein the peptide has the sequence set forth in SEQ ID NO:56.

33. A method of disinfecting a surface of an article or a solution, said method comprising the step of applying to said surface or to said solution an effective amount of a composition comprising at least one microbial peptide of claim 1, wherein said solution optionally further comprises an additional antimicrobial agent.

34. A disinfecting solution comprising at least one microbial peptide claim 1.

35. A peptide comprising the sequence set forth in SEQ ID NO:62 or a derivative thereof, said derivative differing in hydrophobicity and improved in therapeutic index to at least one peptide of SEQ ID NO:1-52, and optionally wherein at least one or each amino acid residue is a D-amino acid residue, wherein the derivative comprises a truncation of at least one or two residues from an end or at least one amino acid residue substitution, wherein the substitution replaces a hydrophilic residue for a hydrophobic residue, wherein the substitution replaces a hydrophobic residue for a hydrophilic residue, wherein the substitution replaces a hydrophilic residue with a different hydrophilic residue, wherein the substitution replaces a hydrophobic residue with a different hydrophobic residue, wherein the substitution replaces an L-residue with a D-residue,
wherein the substitution replaces a D-residue with an L-residue, wherein all amino acid residues are D-residues and wherein there is optionally N-acetylation or covalent linkage at the amino terminus to polyethylene glycol.

36-38. (canceled)
